(19) United States
(12) Patent Application Publication
(10) Pub. No.: US 2024/0246931 A1
(43) Pub. Date: Jul. 25, 2024

(54) COMPOUNDS AS PD1/PD-L1 INHIBITORS AND METHODS THEREOF

(71) Applicant: JUBILANT PRODEL LLC, Bedminster, NJ (US)

(72) Inventors: Naveen SADHU, Bangalore, Karnataka (IN); Dhanalakshmi SIVANANDHAN, Bangalore, Karnataka (IN); Zainuddin MOHD, Bangalore, Karnataka (IN); Sridharan RAJAGOPAL, Bangalore, Karnataka (IN)

(21) Appl. No.: 18/556,846

(22) PCT Filed: Apr. 22, 2022
(86) PCT No.: PCT/IN2022/050381

§ 371 Date: Oct. 23, 2023

(30) Foreign Application Priority Data
Apr. 22, 2021 (IN) 202141018688

(51) Int. Cl. C07D 401/12; A61K 45/06; C07D 205/04; C07D 207/08; C07D 211/22; C07D 221/20; C07D 401/14; C07D 403/12; C07D 413/12; C07D 417/12; C07D 471/04

(52) U.S. Cl. CPC C07D 401/12; A61K 45/06; C07D 205/04; C07D 207/08; C07D 211/22; C07D 221/20; C07D 401/14; C07D 403/12; C07D 413/12; C07D 417/12; C07D 471/04

(57) ABSTRACT

The present invention relates in general, to the field of pharmaceutical compounds, more particularly to the compounds of Formula (I) which acts as inhibitors for PD1/PD-L1 interaction. The present invention further relates to a method of preparation of compounds of Formula (I). The present invention also relates to a composition of compounds of Formula (I).

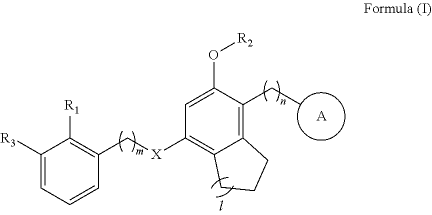

This application claims the benefits of the Indian provisional patent application number 202141018688, filed on 22 Apr. 2021; the specifications of which are hereby incorporated by reference in their entirety and for all purposes.

### FIELD OF INVENTION

The present invention relates in general, to the field of pharmaceutical compounds, more particularly to the compounds of Formula (I) which acts as inhibitors for PD1/PD-L1 interaction. The present invention further relates to a method of preparation of compounds of Formula (I):

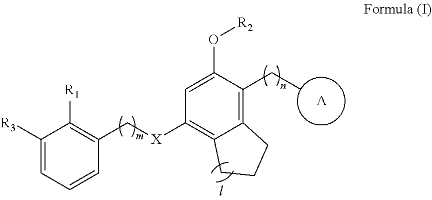

### BACKGROUND

Programmed cell death protein 1 (PD-1) is a protein on the surface of cells that plays a significant role in regulating the immune system in a human body. It provides a response to the cells of the human body by down-regulating the immune system and promoting self-tolerance by suppressing T cell inflammatory activity. Thus PD-1 prevents autoimmune diseases, however, it also prevents the immune system from killing cancer cells. PD-1 has two ligands, PD-L1 (Programmed death-ligand 1) and PD-L2 (Programmed death-ligand 2), which are members of the B7 family. Various evidence suggest that PD-1 and its ligands negatively regulate immune responses. PD-L1 was found to be highly expressed in several cancers and hence the role of PD1 in cancer immune evasion is well established.

In cancer, PD-L1 is expressed on the surface of tumour cells in various solid malignancies such as squamous cell carcinoma of the head and neck, melanoma, carcinomas of the brain, thyroid, thymus, esophagus, lung, breast, gastrointestinal tract, colorectum, liver, pancreas, kidney, etc. (Topalian S. L. et al., Curr. Opin. Immunol., 2012, 24(2):207-212; Wang X. et al., Oncotargets and Therapy, 2016, 9:5023-5039). In hepatocellular carcinoma, melanoma and breast cancer, PD-L1 positivity was correlated with a worse prognosis (Muenst S. et al., Breast Cancer Res. Treat., 2014, 146(1): 15-24; Leung J. et al., Immune Network, 2014, 14(6):265-276; Wang Q. et al., Medicine (Baltimore), 2017, 96(18): e6369). In contrast, normal human tissues seldom express PD-L1 protein on their cell surface, indicating that PD-L1 can be a selective target for anti-tumour therapy (Chen L. et al., J. Clin. Invest., 2015, 125(9):3384-3391).

PD-1/PD-L1 molecular pathway is one of the primary mechanisms of cancer immune evasion. Activation of PD-1/PD-L1 pathway induces apoptosis of activated T cells facilitates T cell energy and exhaustion, enhances the function of regulatory T cells and inhibits the proliferation of T cells. Therefore, blocking this pathway restores the proliferation and cytotoxicity of CTLs, inhibiting the function of regulatory T cells (Tregs), and resulting in a decrease T cell apoptosis.

Blockade of the PD-1/PD-L1 pathway by therapeutic antibodies has been shown to prevent inhibitory signaling from cancer cells and enable CTLs to elicit an immune response against the target/cancer cells. A number of cancer immunotherapy agents targeting PD-1 have been developed till date and approved for a number of malignancies. However, there is still a need for potent and selective small molecule inhibitors of the PD-1/PD-L1 interaction pathway.

Common drug-related adverse effects of both anti-PD-1 and anti-PD-L1 antibodies include diarrhea, pneumonitis, rashes, itchiness, kidney infections and hormonal imbalance. Immune-related adverse effects such as dermatitis, colitis, hepatitis, vitiligo and thyroiditis have also been reported. The long residence time of the monoclonal antibodies (mAbs) could contribute to these AEs, which may be partially circumvented using a small molecule inhibitor. In addition, studies using smaller cell penetrating biologicals and DNA aptamers have shown to exert antibody-mimic functions and are advantageous over antibody for their chemically synthetic nature, low immunogenicity, and efficient tissue penetration (Lai W. Y. et al., Mol. Therapy—Nucl. Acids, 2016, 5: e397). Small molecule inhibitors, therefore, can provide increased oral bioavailability, increased bio-efficiency and shortened half-life activity for a more controllable treatment, particularly in the case of auto-immune or other adverse events.

As discussed, the PD-1/PD-L1 inhibitory compounds have vast utility in upregulating the immune system for efficiently combating cancer. Therefore, the identification of a chemical moiety, especially small molecule inhibitors, that facilitates this inhibition is necessary. Therefore, the identification and development of new PD-1/PD-L1 inhibitor compounds treating cancer and other diseases or conditions associated with activation of PD-1/PD-L1 would open new opportunities in the realm of cancer treatment.

### SUMMARY OF INVENTION

In an aspect of the present invention there is provided a compound of Formula (I):

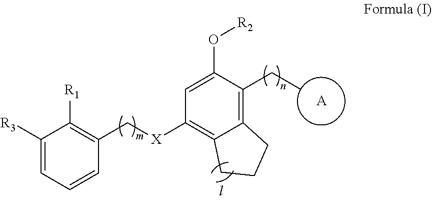

their stereoisomers, an N-oxide or pharmaceutically acceptable salts thereof;
wherein,
X is selected from O or NR′;
Ring A is selected from C₆₋₁₀ aryl, C₃₋₁₀ cycloalkyl, C₇₋₁₆ alkylaryl, C₂₋₁₀ heteroaryl, C₂₋₂₀ heterocyclyl, —CO—C₂₋₂₀ heterocyclyl, or —C(O)NR₄—C₂₋₂₀ heterocyclyl; wherein, C₆₋₁₀ aryl, C₃₋₁₀ cycloalkyl, C₇₋₁₆ alkylaryl, C₂₋₁₀ heteroaryl, C₂₋₂₀ heterocyclyl, —CO—C₂₋₂₀ heterocyclyl, or —C(O)NR₄—C₂₋₂₀ heterocyclyl is optionally substituted with one or more groups selected from halogen, hydroxy, C₁₋₁₀ alkyl, C₁₋₁₀ alkoxy, C₁₋₁₀ haloalkyl, C₂₋₁₀ alkylalkoxy, —CH₂—NRₐC(O)R_{b}, —CRₐR_{b}—OR_{c}, —CRₐR_{b}—NR_{c}R_{d}, or —CH₂—NHC(O)NRₐR_{b}; wherein, Rₐ, R_{b}, R_{c}, and R_{d} are independently selected from hydrogen, halogen, C₁₋₁₀ alkyl, —C(O)R″, C₃₋₁₀ cycloalkyl C₁₋₁₀ haloalkyl, or C₁₋₁₀ alkoxy;
R′ is selected from hydrogen or C₁₋₁₀ alkyl;
R₁ is selected from hydrogen, cyano or C₁₋₁₀ alkyl;
R₂ is selected from hydrogen, C₁₋₁₀ alkyl, C₆₋₁₀ aryl, C₃₋₁₀ cycloalkyl, C₁₋₁₀ haloalkyl, C₇₋₁₆ alkylaryl, C₂₋₁₀ heteroaryl, C₃₋₂₀ alkyl heteroaryl, C₂₋₂₀ heterocyclyl, or C₃₋₂₀ alkyl heterocyclyl; wherein, C₁₋₁₀ alkyl, C₆₋₁₀ aryl, C₃₋₁₀ cycloalkyl, C₁₋₁₀ haloalkyl, C₇₋₁₆ alkylaryl, C₂₋₁₀ heteroaryl, C₃₋₂₀ alkyl heteroaryl, C₂₋₂₀ heterocyclyl, or C₃₋₂₀ alkyl heterocyclyl, is optionally substituted with one or more groups selected from halogen, cyano, hydroxy, —C(O)NH₂, C₁₋₁₀ alkyl, or C₆₋₁₀ aryl;
R₃ is selected from halogen, C₆₋₁₀ aryl, or C₂₋₁₀ heteroaryl; wherein, C₆₋₁₀ aryl, or C₂₋₁₀ heteroaryl, is optionally substituted with one or more groups selected from halogen, haloalkyl, cyano, hydroxy, amino, C₁₋₁₀ alkyl, OR″, C₆₋₁₀ aryl, C₂₋₂₀ heterocyclyl, or C₂₋₁₀ heteroaryl;
wherein, R″ is selected from hydrogen, halogen, C₁₋₁₀ alkyl, or C₁₋₁₀ haloalkyl; R₄ is selected from hydrogen or C₁₋₁₀ alkyl;
mi s 1 to 5; n is 0 to 5; and l is 1 to 5,
provided that the compound of Formula (I) is not:

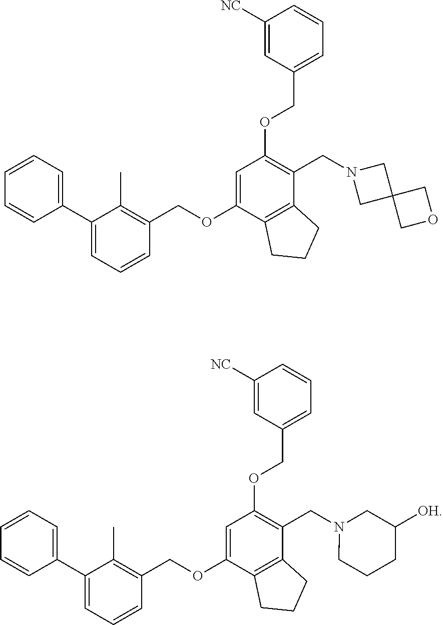 [SMILES: Cc1c(COc2cc(OCc3cccc(C#N)c3)c(CN3CC4(COC4)C3)c3c2CCC3)cccc1-c1ccccc1 ; Cc1c(COc2cc(OCc3cccc(C#N)c3)c(CN3CCCC(O)C3)c3c2CCC3)cccc1-c1ccccc1]

In another aspect of the present invention, there is provided a process for the preparation of compounds of Formula (I), their stereoisomers, an N-oxide or pharmaceutically acceptable salts thereof, comprising the steps of: (a) reacting compounds of Formula (Ia) with a compound A in the presence of a reducing agent and a solvent to obtain compounds of Formula (I):

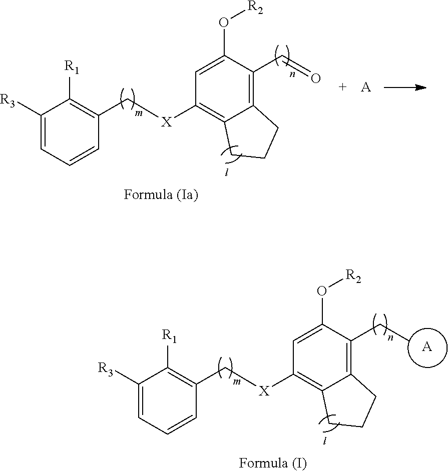

In yet another aspect of the present invention, there is provided a pharmaceutical composition comprising a compound of Formula (I) or a pharmaceutically acceptable salt thereof, together with a pharmaceutically acceptable carrier, optionally in combination with one or more other pharmaceutical compositions.

In another aspect of the present invention, there is provided a method for the treatment and/or prevention of a condition mediated by PD-1/PD-L1 or a proliferative disorder or cancer, comprising administering to a subject suffering from the condition mediated by PD-1/PD-L1 or the proliferative disorder or cancer, a therapeutically effective amount of the compound of Formula (I) or the pharmaceutical composition as disclosed herein.

In another aspect of the present invention, there is provided a compound of Formula (I) or the pharmaceutical composition as disclosed herein, for use in the manufacture of a medicament for inhibiting PD-1/PD-L1 enzymes in a cell.

In yet another aspect of the present invention, there is provided a compound of Formula (I) or the pharmaceutical composition as disclosed herein, for use in the treatment and/or prevention of a condition mediated by PD-1/PD-L1 or a proliferative disorder or cancer, comprising administering to a subject suffering from the condition mediated by PD-1/PD-L1 or the proliferative disorder or cancer.

In one more aspect of the present invention, there is provided use of the compounds of Formula (I), or the pharmaceutical composition, for the treatment or prevention of diseases or proliferative disorder or cancer together with other clinically relevant cytotoxic agents or non-cytotoxic agents.

In a further aspect of the present invention, there is provided a method for the treatment of cancer, said method comprising administering a combination of the compounds of Formula (I) or the pharmaceutical composition as disclosed herein, with other clinically relevant cytotoxic agents or non-cytotoxic agents to a subject in need thereof.

These and other features, aspects, and advantages of the present subject matter will become better understood with reference to the following description. This summary is provided to introduce a selection of concepts in a simplified form. This summary is not intended to identify key features or essential features of the invention, nor is it intended to be used to limit the scope of the subject matter.

### DETAILED DESCRIPTION OF THE INVENTION

Those skilled in the art will be aware that the present invention is subject to variations and modifications other than those specifically described. It is to be understood that the present invention includes all such variations and modifications. The invention also includes all such steps, features, compositions and compounds referred to or indicated in this specification, individually or collectively, and any and all combinations of any or more of such steps or features.

### Definitions

For convenience, before further description of the present invention, certain terms employed in the specification, and examples are collected here. These definitions should be read in the light of the remainder of the invention and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The term “compound(s)” comprises the compounds disclosed in the present invention.

As used herein, the term “or” means “and/or” unless stated otherwise.

The terms “comprise” and “comprising” are used in the inclusive, open sense, meaning that additional elements may be included. Throughout this specification, unless the context requires otherwise the word “comprise”, and variations, such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

In the structural formulae given herein and throughout the present invention, the following terms have been indicated meaning, unless specifically stated otherwise.

Furthermore, the compound of Formula (I) can be its derivatives, analogs, tautomeric forms, enantiomers, diastereomers, geometrical isomers, polymorphs, solvates, intermediates, metabolites, prodrugs or pharmaceutically acceptable salts, and compositions.

The compounds described herein may contain one or more chiral centers and/or double bonds and therefore, may exist as stereoisomers, such as double-bond isomers (i.e., geometric isomers), regioisomers, enantiomers or diastereomers. Accordingly, the chemical structures depicted herein encompass all possible enantiomers and stereoisomers of the illustrated or identified compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the person skilled in the art. The compounds may also exist in several tautomeric forms including the enol form, the keto form and mixtures thereof. Accordingly, the chemical structures depicted herein encompass all possible tautomeric forms of the illustrated or identified compounds. It is also understood that some isomeric form such as diastereomers, enantiomers and geometrical isomers can be separated by physical and/or chemical methods and by those skilled in the art. Pharmaceutically acceptable solvates may be hydrates or comprising of other solvents of crystallization such as alcohols, ether, and the like.

According to the present invention, the compounds provided herein, includes all of the corresponding enantiomers and stereoisomers, that is, the pure form of the stereoisomers, in terms of geometrical isomer, enantiomer, or diastereomer, and the mixture of enantiomeric and stereoisomeric form of said compounds. Further, the mixture of enantiomeric and stereoisomeric forms can be resolved into their pure component by the methods known in the art, such as chiral-phase gas chromatography, chiral-phase high performance liquid chromatography, crystallization, using chiral derivatizing agents, etc. Also, the pure enantiomers and stereoisomers can be obtained from intermediates or metabolites and reagents that are in the form of pure enantiomers and stereoisomers by known asymmetric synthetic methods.

The term “pharmaceutically acceptable” refers to compounds or compositions that are physiologically tolerable and do not typically produce allergic or similar untoward reactions, including but not limited to gastric upset or dizziness when administered to subjects.

Pharmaceutically acceptable salts forming part of this invention include salts derived from inorganic bases such as Li, Na, K, Ca, Mg, Fe, Cu, Zn, Mn, ammonium, substituted ammonium salts, aluminum salts, and the like.; salts of organic bases such as N, N′-diacetylethylenediamine, glucamine, triethylamine, choline, dicyclohexylamine, benzylamine, trialkylamine, thiamine, guanidine, diethanolamine, α-phenylethylamine, piperidine, morpholine, pyridine, hydroxyethylpyrrolidine, hydroxyethylpiperidine and the like, salts also include amino acid salts such as glycine, alanine, cystine, cysteine, lysine, arginine, phenylalanine, guanidine, etc. Salts may include acid addition salts where appropriate which are sulphates, nitrates, phosphates, perchlorates, borates, hydrohalides, acetates, tartrates, maleates, fumarates, formates, citrates, succinates, lactates, mesylates, trifluoroacetates, acetates, besylates, propionates, mandelates, hydrobromides, hydrochlorides, palmoates, methanesulphonates, tosylates, benzoates, salicylates, hydroxynaphthoates, benzenesulfonates, ascorbates, glycerophosphates, ketoglutarates and the like.

The term “intermediate” refers to the compounds with same core structure of the compounds of the Formula (I) varying at specific allowed positions (for example alkyl chains).

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents, for example, include those described herein above. The permissible substituents can be one or more and the same or different for appropriate organic compounds. For purposes of this invention, the heteroatoms such as nitrogen may have hydrogen substituents, and/or any permissible substituents of organic compounds described herein which satisfy the valences of the heteroatoms. It is understood that the substituent may be further substituted.

The term “alkyl” refers to straight or branched aliphatic hydrocarbon groups having the specified number of carbon atoms, which are attached to the rest of the molecule by a single atom, which may be optionally substituted by one or more substituents. Preferred alkyl groups include, without limitation, methyl, ethyl, n-propyl, isopropyl, butyl, isobutyl, t-butyl, pentyl, hexyl, heptyl, octyl and the like.

The term “cycloalkyl” refers to non-aromatic mono or polycyclic ring system of about 3 to 10 carbon atoms, which may be optionally substituted by one or more substituents. The polycyclic ring denotes hydrocarbon systems containing two or more ring systems with one or more ring carbon atoms in common i.e. a spiro, fused or bridged structures. Preferred cycloalkyl groups include, without limitation, cyclopropyl, cyclobutyl, cyclopentyl, cyclohexyl, cyclooctanyl, bridged cyclic groups or spirobicyclic groups e.g. spiro [4.4] non-2-yl and the like.

The term “alkoxy” refers to an alkyl group attached via an oxygen linkage to the rest of the molecule, which may be optionally substituted by one or more substituents. Alkoxy groups refer to compounds with 1 to 10 carbon atoms and preferred alkoxy groups include, without limitation, —OCH₃, —OC₂H₅ and the like.

The term “halo” or “halogen” alone or in combination with other term(s) means fluorine, chlorine, bromine or iodine.

The term “amino” refers to —NH₂ group.

The term “hydroxy/hydroxyl” refers to —OH group.

The term “oxo” refers to a ═O group.

The term “cyano” refers to a —CN group.

The term “heteroatom” as used herein designates a sulfur, nitrogen or oxygen atom.

The term “haloalkyl” refers to alkyl with one or more halogen atoms. In the present invention, the term haloalkyl refers to compounds with 1 to 10 carbon atoms and examples of haloalkyl include but are not limited to —CH₂F, —CHF₂, —CF₃, —C₂H₄F and the like.

The term “aryl” refers to aromatic radicals having 6 to 10 carbon atoms, which may be optionally substituted by one or more substituents. Preferred aryl groups include not limited to phenyl and the like.

The term “heteroaryl” refers to an aromatic heterocyclic ring radical as defined above. The heteroaryl ring radical may be attached to the main structure at any heteroatom or carbon atom that results in the creation of a stable structure. The heteroaryl refers to aromatic ring with one or more hetero atoms selected from N, O or S with carbon ranging between 2 to 10.

The term “heterocyclyl” refers to a heterocyclic ring radical that may be optionally substituted by one or more substituents. The heterocyclyl ring radical may be attached to the main structure at any heteroatom or carbon atom that results in the creation of a stable structure.

Furthermore, the term “heterocyclyl” refers to a stable 2 to 20 membered rings radical, which consists of carbon atoms and heteroatoms selected from nitrogen, phosphorus, oxygen, and sulfur. For purposes of this invention the heterocyclic ring radical may be monocyclic, bicyclic or tricyclic ring systems, and the nitrogen, phosphorus, carbon, or sulfur atoms in the heterocyclic ring radical may be optionally oxidized to various oxidation states. In addition, the nitrogen atom may be optionally quaternized; and the ring radical may be partially or fully saturated. Preferred heterocyclyl groups include, without limitation, azetidinyl, acridinyl, benzodioxolyl, benzodioxanyl, benzofuranyl, carbazolyl, cinnolinyl, dioxolanyl, indolizinyl, naphthyridinyl, perhydroazepinyl, phenazinyl, phenothiazinyl, phenoxazinyl, phthalazinyl, pyridyl, pteridinyl, purinyl, quinazolinyl, quinoxalinyl, quinolinyl, isoquinolinyl, tetrazolyl, imidazolyl, tetrahydroisoquinolinyl, piperidinyl, piperazinyl, homopiperazinyl, 2-oxoazepinyl, azepinyl, pyrrolyl, 4-piperidonyl, pyrrolidinyl, pyrazinyl, pyrimidinyl, pyridazinyl, oxazolyl, oxazolinyl, triazolyl, indanyl, isoxazolyl, isoxazolidinyl, thiazolyl, thiazolinyl, thiazolidinyl, isothiazolyl, quinuclidinyl, isothiazolidinyl, indolyl, isoindolyl, indolinyl, isoindolinyl, octahydroindolyl, octahydroisoindolyl, quinolyl, isoquinolyl, decahydroisoquinolyl, benzimidazolyl, thiadiazolyl, benzopyranyl, benzothiazolyl, benzooxazolyl, thienyl, morpholinyl, thiomorpholinyl, thiamorpholinyl sulfoxide, furyl, tetrahydrofuryl, tetrahydropyranyl, chromanyl, and isochromanyl.

The term “heterocyclyl” refers to monocyclic or polycyclic ring, polycyclic ring system refers to a ring system containing 2 or more rings, preferably bicyclic or tricyclic rings, in which rings can be fused, bridged or spiro rings or any combinations thereof. A fused ring as used herein means that the two rings are linked to each other through two adjacent ring atoms common to both rings. The fused ring can contain 1-4 hetero atoms independently selected from N, O, or S. The rings can be either fused by nitrogen or —CH— group.

The term “alkylaryl” refers to an aryl group directly bonded to an alkyl group, which may be optionally substituted by one or more substituents. For the purpose of the present invention, the arylalkyl group of the present invention refers to compounds with carbon atoms ranging between 7 to 16, which includes alkyl group with 1 to 6 carbon atoms and aryl ring with 6 to 10 carbon atoms. Preferred alkylaryl groups include, without limitation, —CH₂-phenyl, —C₂H₄-phenyl, C₃H₆-phenyl and the like.

The term “arylalkyl” refers to an aryl group directly bonded to an alkyl group, which may be optionally substituted by one or more substituents. For the purpose of the present invention, the arylalkyl group of the present invention refers to compounds with carbon atoms ranging between 7 to 16, which includes the aryl ring with 6 to 10 carbon atoms and alkyl group with 1 to 6 carbon atoms. Preferred arylalkyl groups include, without limitation, —C₆H₅—CH₂—, —C₆H₅-C₂H₄— and the like.

The term “alkylalkoxy” refers to an alkyl group attached to an alkoxy group. For the purpose of the present invention, the term alkylalkoxy group refers to compounds with carbon atoms ranging between 2 to 10, which includes the alkyl group with 1 to 9 carbon atoms and an alkoxy group with 1 to 9 carbon atoms but total number of carbons in the range of 2 to 10.

The term “alkylheteroaryl” refers to alkyl attached to heteroaryl group and may be optionally substituted. For the purpose of the present invention, the alkyl heteroaryl refers to compounds with carbon atoms ranging between 3 to 20, which includes the alkyl group with 1 to 10 carbon atoms and heteroaryl ring with 2 to 10 carbon atoms having one or more heteroatoms selected from N, O or S.

The term “alkyl heterocyclyl” refers to alkyl attached to heterocyclyl group and may be optionally substituted. For the purpose of the present invention, the term “alkyl heterocyclyl” refers to compounds with carbon atoms ranging between 2 to 20, which includes the alkyl group with 1 to 10 carbon atoms and heterocyclyl ring with 1 to 10 carbon atoms having one or more heteroatoms selected from N, O or S. The heterocyclyl ring may be bridged, fused or spiral ring as defined herein.

Certain of the compounds disclosed herein can exist as N-oxides. For example, it is known that the pyrazoles can form N-oxides on treatment with a suitable oxidizing agent. Similarly, it is known that the pyridine ring nitrogen can be oxidized on treatment with a suitable oxidizing agent to form an N-oxide.

It is understood that included in the family of compounds of Formula (I) are isomeric forms including diastereomers, enantiomers, tautomers, and geometrical isomers in “E” or “Z” configurational isomer or a mixture of ‘E’ and ‘Z’ isomers. It is also understood that some isomeric form such as diastereomers, enantiomers and geometrical isomers can be separated by physical and/or chemical methods and by those skilled in the art.

Compounds disclosed herein may exist as single stereoisomers, and or mixtures of enantiomers and/or diastereomers. All such single stereoisomers, and mixtures thereof are intended to be within the scope of the subject matter described.

Compounds disclosed herein include isotopes of hydrogen, carbon, oxygen, fluorine, chlorine, iodine and sulfur which can be incorporated into the compounds, such as not limited to ²H (D), ³H (T), ¹¹C, ¹³C, ¹⁴C, ¹⁵N, ¹⁸F, ³⁵S, ³⁶Cl and ¹²⁵I. Compounds of this invention wherein atoms were isotopically labeled for example radioisotopes such as ³H, ¹³C, ¹⁴C, and the like can be used in metabolic studies, kinetic studies. Compounds of the invention where hydrogen is replaced with deuterium may improve the metabolic stability and pharmacokinetics properties of the drug such as in vivo half-life.

Described herein are prodrugs of the compound of Formula (I), which on administration undergo chemical conversion by metabolic processes before becoming active pharmacological substances. In general, such prodrugs will be functional derivatives of a compound of the invention, which are readily convertible in vivo into a compound of the invention.

The compounds described herein can also be prepared in any solid or liquid physical form, for example, the compound can be in a crystalline form, in amorphous form and have any particle size. Furthermore, the compound particles may be micronized or nanonized, or may be agglomerated, particulate granules, powders, oils, oily suspensions or any other form of solid or liquid physical forms.

The compounds described herein may also exhibit polymorphism. This invention further includes different polymorphs of the compounds of the present invention. The term polymorph refers to a particular crystalline state of a substance, having particular physical properties such as X-ray diffraction, IR spectra, melting point and the like.

The term “PD-1/PD-L1 inhibitor or inhibitory compounds” or “inhibitors of PD-1/PD-L1 activation” is used to identify a compound, which is capable of blocking PD-1/PD-L1 pathway to prevent inhibitory signaling from cancer cells and enabling CTLs to elicit an immune response against the target/cancer cells and thus treat cancer and other diseases or conditions associated with activation of PD1/PD-L1.

The term “cytotoxic agents” or “inhibitors” is used to identify any agents or drugs which are capable of killing cells including cancer cells. These agents or inhibitors may stop cancer cells from growing and dividing and may cause tumors to shrink in size.

The term “non-cytotoxic agents” or “inhibitors” is used to identify any agents or inhibitors are which do not directly kill cells, but instead affect cellular transport and metabolic functions to ultimately produce cell death.

The term “immune checkpoint inhibitors agents” or “immune modulators agents” are used to identify any agents or inhibitors that block certain proteins made by some types of immune system cells, such as T cells, and some cancer cells. These proteins help keep immune responses in check and can keep T cells from killing cancer cells. When these proteins are blocked, the “brakes” on the immune system are released and T cells can kill cancer cells better. The immune checkpoint inhibitors include inhibitors against immune checkpoint molecules such as CD27, CD28, CD40, CD 122, CD96, CD73, CD47, OX40, GITR, CSF1R, JAK, PI3K delta, PI3K gamma, TAM arginase, CD137 (also known as 4-1B), ICOS, A2AR, B7-H3, B7-H4, BTLA, CTLA-4, LAG3, TIM3, VISTA, PD-1, PD-L1 and PD-L2. The terms “immune modulators agents” and “immune checkpoint inhibitors” are used interchangeably throughout the present invention.

The term “composition” is intended to encompass a product comprising the specified ingredients in the specified amounts, as well as any product which results, directly or indirectly, from a combination of the specified ingredients in the specified amounts. By “pharmaceutically acceptable” it is meant the carrier, diluent or excipient must be compatible with the other ingredients of the formulation and not deleterious to the recipient thereof.

The term “pharmaceutical composition” refers to a composition(s) containing a therapeutically effective amount of at least one compound of Formula (I) or its pharmaceutically acceptable salt; and a conventional pharmaceutically acceptable carrier.

The pharmaceutical composition(s) of the present invention can be administered orally, for example in the form of tablets, coated tablets, pills, capsules, granules or elixirs. Administration, however, can also be carried out rectally, for example in the form of suppositories, or parenterally, for example intravenously, intramuscularly or subcutaneously, in the form of injectable sterile solutions or suspensions, or topically, for example in the form of ointments or creams or transdermals, in the form of patches, or in other ways, for example in the form of aerosols or nasal sprays.

The pharmaceutical composition(s) usually contain(s) about 1% to 99%, for example, about 5% to 75%, or from about 10% to about 30% by weight of the compound of Formula (I) or pharmaceutically acceptable salts thereof. The amount of the compound of Formula (I) or pharmaceutically acceptable salts thereof in the pharmaceutical composition(s) can range from about 1 mg to about 1000 mg or from about 2.5 mg to about 500 mg or from about 5 mg to about 250 mg or in any range falling within the broader range of 1 mg to 1000 mg or higher or lower than the afore mentioned range.

The term “treat”, “treating” and “treatment” refer to any treatment of a disease in a mammal, including: (a) Inhibiting the disease, i.e., slowing or arresting the development of clinical symptoms; and/or (b) relieving the disease, i.e., causing the regression of clinical symptoms and/or (c) alleviating or abrogating a disease and/or its attendant symptoms.

The term “prevent”, “preventing” and “prevention” refer to a method of preventing the onset of a disease and/or its attendant symptoms or barring a subject from acquiring a disease. As used herein, “prevent”, “preventing” and “prevention” also include delaying the onset of a disease and/or its attendant symptoms and reducing a subject's risk of acquiring a disease.

The term “therapeutically effective amount” refers to that amount of a compound of Formula (I) or a pharmaceutically acceptable salt or a stereoisomer thereof; or a composition comprising the compound of Formula (I) or a pharmaceutically acceptable salt or a stereoisomer thereof, effective in producing the desired therapeutic response in a particular patient suffering from a diseases or disorder, in particular their use in diseases or disorder associated with cancer. Particularly, the term “therapeutically effective amount” includes the amount of the compound of Formula (I) or a pharmaceutically acceptable salt or a stereoisomer thereof, when administered, that induces a positive modification in the disease or disorder to be treated or is sufficient to prevent development of, or alleviate to some extent, one or more of the symptoms of the disease or disorder being treated in a subject. In respect of the therapeutic amount of the compound, the amount of the compound used for the treatment of a subject is low enough to avoid undue or severe side effects, within the scope of sound medical judgment can also be considered. The therapeutically effective amount of the compound or composition will be varied with the particular condition being treated, the severity of the condition being treated or prevented, the duration of the treatment, the nature of concurrent therapy, the age and physical condition of the end user, the specific compound or composition employed the particular pharmaceutically acceptable carrier utilized.

A term once described, the same meaning applies for it, throughout the patent.

As discussed in the background, the identification and development of new PD-1/PD-L1 inhibitor compounds treating cancer and other diseases or conditions associated with activation of PD-1/PD-L1 would open wide opportunities in the treatment of diseases, conditions, or cancer associated with PD-1/PD-L1.

In an implementation of the present invention, there is provided a compound of Formula (I),

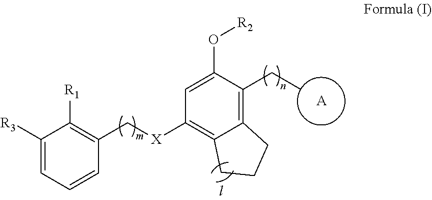

their stereoisomers, an N-oxide or pharmaceutically acceptable salts thereof;
wherein, X is selected from O or NR′;
Ring A is selected from C₆₋₁₀ aryl, C₃₋₁₀ cycloalkyl, C₇₋₁₆ alkylaryl, C₂₋₁₀ heteroaryl, C₂₋₂₀ heterocyclyl, —CO—C₂₋₂₀ heterocyclyl, or —C(O)NR₄-C₂₋₂₀ heterocyclyl; wherein, C₆₋₁₀ aryl, C₃₋₁₀ cycloalkyl, C₇₋₁₆ alkylaryl, C₂₋₁₀ heteroaryl, C₂₋₂₀ heterocyclyl, —CO—C₂₋₂₀ heterocyclyl, or —C(O)NR₄—C₂₋₂₀ heterocyclyl is optionally substituted with one or more groups selected from halogen, hydroxy, C₁₋₁₀ alkyl, C₁₋₁₀ alkoxy, C₁₋₁₀ haloalkyl, C₂₋₁₀ alkylalkoxy, —CH₂—NRₐC(O)R_{b}, —CRₐR_{b}—OR_{c}, —CRₐR_{b}—NR_{c}R_{d}, or —CH₂—NHC(O)NRₐR_{b}; wherein, Rₐ, R_{b}, R_{c}, and R_{d} are independently selected from hydrogen, halogen, C₁₋₁₀ alkyl, —C(O)R″, C₃₋₁₀ cycloalkyl C₁₋₁₀ haloalkyl, or C₁₋₁₀ alkoxy;
R′ is selected from hydrogen or C₁₋₁₀ alkyl;
R₁ is selected from hydrogen, cyano or C₁₋₁₀ alkyl;
R₂ is selected from hydrogen, C₁₋₁₀ alkyl, C₆₋₁₀ aryl, C₃₋₁₀ cycloalkyl, C₁₋₁₀ haloalkyl, C₇₋₁₆ alkylaryl, C₂₋₁₀ heteroaryl, C₃₋₂₀ alkyl heteroaryl, C₂₋₂₀ heterocyclyl, or C₃₋₂₀ alkyl heterocyclyl; wherein, C₁₋₁₀ alkyl, C₆₋₁₀ aryl, C₃₋₁₀ cycloalkyl, C₁₋₁₀ haloalkyl, C₇₋₁₆ alkylaryl, C₂₋₁₀ heteroaryl, C₃₋₂₀ alkyl heteroaryl, C₂₋₂₀ heterocyclyl, or C₃₋₂₀ alkyl heterocyclyl, is optionally substituted with one or more groups selected from halogen, cyano, hydroxy, —C(O)NH₂, C₁₋₁₀ alkyl, or C₆₋₁₀ aryl;
R₃ is selected from halogen, C₆₋₁₀ aryl, or C₂₋₁₀ heteroaryl; wherein, C₆₋₁₀ aryl, or C₂₋₁₀ heteroaryl, is optionally substituted with one or more groups selected from halogen, haloalkyl, cyano, hydroxy, amino, C₁₋₁₀ alkyl, OR″, C₆₋₁₀ aryl, C₂₋₂₀ heterocyclyl, or C₂₋₁₀ heteroaryl;
R″ is selected from hydrogen, halogen, C₁₋₁₀ alkyl, or C₁₋₁₀ haloalkyl;
R₄ is selected from hydrogen or C₁₋₁₀ alkyl;
m is 1 to 5; n is 0 to 5; and l is 1 to 5,
provided that the compound of Formula (I) is not:

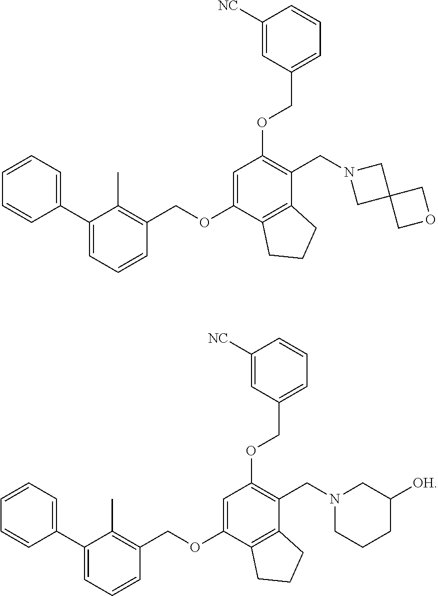 [SMILES: Cc1c(COc2cc(OCc3cccc(C#N)c3)c(CN3CC4(COC4)C3)c3c2CCC3)cccc1-c1ccccc1 ; Cc1c(COc2cc(OCc3cccc(C#N)c3)c(CN3CCCC(O)C3)c3c2CCC3)cccc1-c1ccccc1]

In an implementation of the present invention, there is provided a compound of Formula (I), their stereoisomers, an N-oxide or pharmaceutically acceptable salts thereof; wherein, X is selected from O;
Ring A is selected from C₆₋₁₀ aryl, C₃₋₁₀ cycloalkyl, C₇₋₁₆ alkylaryl, C₂₋₁₀ heteroaryl, C₂₋₂₀ heterocyclyl, —CO—C₂₋₂₀ heterocyclyl, or —C(O)NR₄—C₂₋₂₀ heterocyclyl; wherein, C₆₋₁₀ aryl, C₃₋₁₀ cycloalkyl, C₇₋₁₆ alkylaryl, C₂₋₁₀ heteroaryl, C₂₋₂₀ heterocyclyl, —CO—C₂₋₂₀ heterocyclyl, or —C(O)NR₄—C₂₋₂₀ heterocyclyl is optionally substituted with one or more groups selected from halogen, hydroxy, C₁₋₁₀ alkyl, C₁₋₁₀ alkoxy, C₁₋₁₀ haloalkyl, —CH₂—NRₐC(O)R_{b}, —CRₐR_{b}—OR_{c}, —CRₐR_{b}—NR_{c}R_{d}, or —CH₂—NHC(O)NRₐR_{b}; wherein, Rₐ, R_{b}, R_{c}, and R_{d} are independently selected from hydrogen, halogen, C₁₋₁₀ alkyl, —C(O)R″, C₃₋₁₀ cycloalkyl C₁₋₁₀ haloalkyl, or C₁₋₁₀ alkoxy;
R₁ is selected from hydrogen, cyano or C₁₋₁₀ alkyl;
R₂ is selected from hydrogen, C₁₋₁₀ alkyl, C₆₋₁₀ aryl, C₃₋₁₀ cycloalkyl, C₁₋₁₀ haloalkyl, C₇₋₁₆ alkylaryl, C₂₋₁₀ heteroaryl, C₃₋₂₀ alkyl heteroaryl, C₂₋₂₀ heterocyclyl, or C₃₋₂₀ alkyl heterocyclyl; wherein, C₁₋₁₀ alkyl, C₆₋₁₀ aryl, C₃₋₁₀ cycloalkyl, C₁₋₁₀ haloalkyl, C₇₋₁₆ alkylaryl, C₂₋₁₀ heteroaryl, C₃₋₂₀ alkyl heteroaryl, C₂₋₂₀ heterocyclyl, or C₃₋₂₀ alkyl heterocyclyl, is optionally substituted with one or more groups selected from halogen, cyano, hydroxy, —C(O)NH₂, C₁₋₁₀ alkyl, or C₆₋₁₀ aryl;
R₃ is selected from halogen, C₆₋₁₀ aryl, or C₂₋₁₀ heteroaryl; wherein, C₆₋₁₀ aryl, or C₂₋₁₀ heteroaryl, is optionally substituted with one or more groups selected from halogen, haloalkyl, cyano, hydroxy, amino, C₁₋₁₀ alkyl, OR″, C₆₋₁₀ aryl, C₂₋₂₀ heterocyclyl, or C₂₋₁₀ heteroaryl;
wherein, R″ is selected from hydrogen, halogen, C₁₋₁₀ alkyl, or C₁₋₁₀ haloalkyl;
R₄ is selected from hydrogen or C₁₋₁₀ alkyl;
m is 1 to 5; n is 0 to 5; and l is 1 to 5.

In an implementation of the present invention, there is provided the compound of Formula (I), their stereoisomers, an N-oxide or pharmaceutically acceptable salts thereof; wherein, X is O; R₁ is cyano or C₁₋₆ alkyl; R₂ is selected from C₁₋₆ haloalkyl, C₆₋₁₀ aryl, C₇₋₁₂ alkylaryl, C₃₋₁₆ alkyl heteroaryl, or C₃₋₂₀ alkyl heterocyclyl, wherein C₁₋₆ haloalkyl, C₆₋₁₀ aryl, C₇₋₁₂ alkylaryl, C₃₋₁₆ alkyl heteroaryl, or C₃₋₂₀ alkyl heterocyclyl is optionally substituted with one or more groups selected from C₁₋₆ alkyl, cyano, hydroxy, or —C(O)NH₂; R₃ is halogen, C₆₋₈ aryl, or C₂₋₁₀ heteroaryl; wherein, C₆₋₈ aryl, or C₂₋₁₀ heteroaryl, is optionally substituted with one or more groups selected from halogen, haloalkyl, hydroxy, amino, C₁₋₁₀ alkyl, OR″ or C₂₋₂₀ heterocyclyl; Ring A is selected from C₂₋₁₀ heterocyclyl, CO—C₂₋₁₀ heterocyclyl or —C(O)NR₄—C₂₋₁₀ heterocyclyl; wherein, C₂₋₁₀ heterocyclyl, CO—C₂₋₁₀ heterocyclyl or —C(O)NR₄—C₂₋₁₀ heterocyclyl is optionally substituted with one or more groups selected from halogen, hydroxy, C₁₋₆ alkyl, —CH₂—NRₐC(O)R_{b}, —CRₐR_{b}—OR_{c}, —CRₐR_{b}—NR_{c}R_{d}, or —CH₂—NHC(O)NRₐR_{b}; wherein, Rₐ, R_{b}, R_{c}, and R_{d} are independently selected from hydrogen, C₁₋₆ alkyl, or —C(O)R″; R₄ is hydrogen; and n is 0 to 1.

In an implementation of the present invention, there is provided the compound of Formula (I), their stereoisomers, an N-oxide or pharmaceutically acceptable salts thereof; wherein, X is O; R₁ is C₁₋₆ alkyl; R₂ is C₃₋₁₀ alkyl heteroaryl; wherein, C₃₋₁₀ alkyl heteroaryl is optionally substituted with one or more groups selected from C₁₋₆ alkyl or cyano; R₃ is C₆₋₈ aryl; Ring A is C₂₋₁₀ heterocyclyl; optionally substituted with —CH₂OR_{c}; wherein, R_{c} is hydrogen; m is 1; n is 1; and l is 1.

In an implementation of the present invention, there is provided the compound of Formula (I), their stereoisomers, an N-oxide or pharmaceutically acceptable salts thereof; wherein, m is 1 to 2; n is 0 to 2; and l is 1 to 2. In another implementation of the present invention, there is provided the compound of Formula (I), their stereoisomers, an N-oxide or pharmaceutically acceptable salts thereof, wherein m is 1; n is 1; and l is 1.

In an implementation of the present invention, there is provided compound of Formula (I), their stereoisomers, an N-oxide or pharmaceutically acceptable salts thereof, wherein, A is selected from:

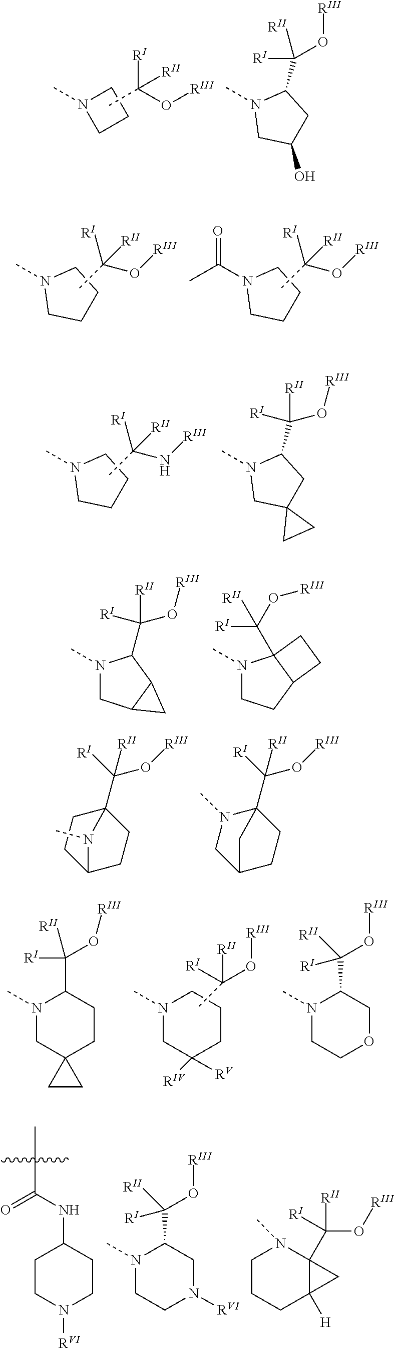

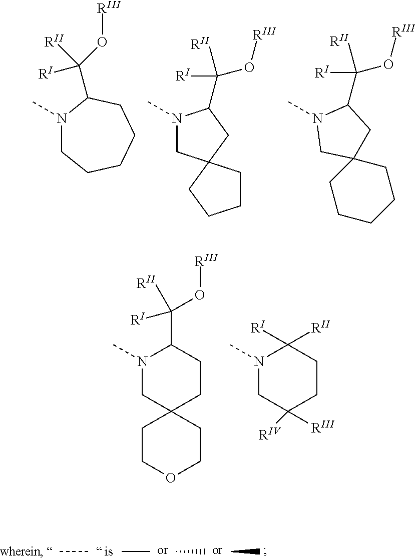

R^{I}, R^{II}, R^{III}, R^{IV}, R^{V} and R^{VI} are independently selected from hydrogen, C₁₋₁₀ alkyl, —C(O)R″, —C(O)NH—R″, —CH₂—OR″, halogen or C₁₋₁₀ haloalkyl.

In an implementation of the present invention, there is provided a compound of Formula (II),

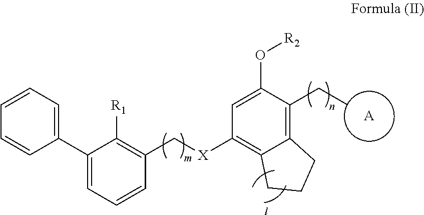

their stereoisomers, an N-oxide or pharmaceutically acceptable salts thereof;
wherein,
X is selected from O or NR′;
Ring A is selected from C₆₋₁₀ aryl, C₃₋₁₀ cycloalkyl, C₇₋₁₆ alkylaryl, C₂₋₁₀ heteroaryl, C₂₋₂₀ heterocyclyl, —CO—C₂₋₂₀ heterocyclyl, or —C(O)NR₄-C₂₋₂₀ heterocyclyl; wherein, C₆₋₁₀ aryl, C₃₋₁₀ cycloalkyl, C₇₋₁₆ alkylaryl, C₂₋₁₀ heteroaryl, C₂₋₂₀ heterocyclyl, —CO—C₂₋₂₀ heterocyclyl, or —C(O)NR₄—C₂₋₂₀ heterocyclyl is optionally substituted with one or more groups selected from halogen, hydroxy, C₁₋₁₀ alkyl, C₁₋₁₀ alkoxy, C₁₋₁₀ haloalkyl, C₂₋₁₀ alkylalkoxy, —CH₂—NRₐC(O)R_{b}, —CRₐR_{b}—OR_{c}, —CRₐR_{b}—NR_{c}R_{d}, or —CH₂—NHC(O)NRₐR_{b}; wherein, Rₐ, R_{b}, R_{c}, and R_{d} are independently selected from hydrogen, halogen, C₁₋₁₀ alkyl, —C(O)R″, C₃₋₁₀ cycloalkyl C₁₋₁₀ haloalkyl, or C₁₋₁₀ alkoxy;
R′ is selected from hydrogen or C₁₋₁₀ alkyl;
R₁ is selected from hydrogen, cyano or C₁₋₁₀ alkyl;
R₂ is selected from hydrogen, C₁₋₁₀ alkyl, C₆₋₁₀ aryl, C₃₋₁₀ cycloalkyl, C₁₋₁₀ haloalkyl, C₇₋₁₆ alkylaryl, C₂₋₁₀ heteroaryl, C₃₋₂₀ alkyl heteroaryl, C₂₋₂₀ heterocyclyl, or C₃₋₂₀ alkyl heterocyclyl; wherein, C₁₋₁₀ alkyl, C₆₋₁₀ aryl, C₃₋₁₀ cycloalkyl, C₁₋₁₀ haloalkyl, C₇₋₁₆ alkylaryl, C₂₋₁₀ heteroaryl, C₃₋₂₀ alkyl heteroaryl, C₂₋₂₀ heterocyclyl, or C₃₋₂₀ alkyl heterocyclyl, is optionally substituted with one or more groups selected from halogen, cyano, hydroxy, —C(O)NH₂, C₁₋₁₀ alkyl, or C₆₋₁₀ aryl;
R″ is selected from hydrogen, halogen, C₁₋₁₀ alkyl, or C₁₋₁₀ haloalkyl;
R₄ is selected from hydrogen or C₁₋₁₀ alkyl;
m is 1 to 5; n is 0 to 5; and l is 1 to 5.

In an implementation of the present invention, there is provided a compound of Formula (IA):

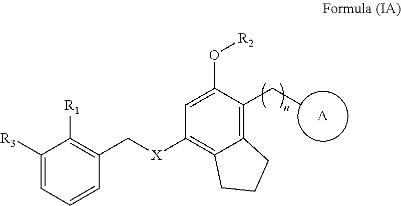

their stereoisomers, an N-oxide or pharmaceutically acceptable salts thereof;
wherein,
X is selected from O;
Ring A is selected from C₂₋₂₀ heterocyclyl, —CO—C₂₋₂₀ heterocyclyl, or —C(O)NR₄-C₂₋₂₀ heterocyclyl; wherein, C₂₋₂₀ heterocyclyl, —CO—C₂₋₂₀ heterocyclyl, or —C(O)NR₄—C₂₋₂₀ heterocyclyl is optionally substituted with one or more groups selected from halogen, hydroxy, C₁₋₁₀ alkyl, —CH₂—NRₐC(O)R_{b}, —CRₐR_{b}—OR_{c}, —CRₐR_{b}—NR_{c}R_{d}, or —CH₂—NHC(O)NRₐR_{b}; wherein, Rₐ, R_{b}, R_{c}, and R_{d} are independently selected from hydrogen, C₁₋₁₀ alkyl, or —C(O)R″;
R₁ is selected from cyano or C₁₋₁₀ alkyl;
R₂ is selected from C₆₋₁₀ aryl, C₁₋₁₀ haloalkyl, C₇₋₁₆ alkylaryl, C₃₋₂₀ alkyl heteroaryl or C₃₋₂₀ alkyl heterocyclyl; wherein, C₆₋₁₀ aryl, C₁₋₁₀ haloalkyl, C₇₋₁₆ alkylaryl, C₃₋₂₀ alkyl heteroaryl, or C₃₋₂₀ alkyl heterocyclyl, is optionally substituted with one or more groups selected from cyano, hydroxy, —C(O)NH₂, or C₁₋₁₀ alkyl;
R₃ is selected from halogen, C₆₋₁₀ aryl, or C₂₋₁₀ heteroaryl; wherein, C₆₋₁₀ aryl, or C₂₋₁₀ heteroaryl, is optionally substituted with one or more groups selected from halogen, haloalkyl, hydroxy, amino, C₁₋₁₀ alkyl, OR″ or C₂₋₂₀ heterocyclyl;
R″ is selected from C₁₋₁₀ alkyl, or C₁₋₁₀ haloalkyl;
R₄ is hydrogen; and
n is 0 to 1.

In an implementation of the present invention, there is provided a compound of Formula (IA):

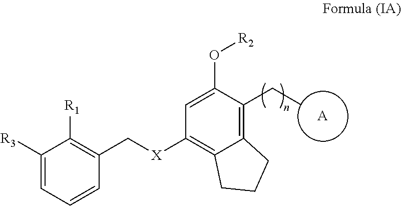

their stereoisomers, an N-oxide or pharmaceutically acceptable salts thereof;
wherein,
X is selected from O;
Ring A is selected from C₂₋₂₀ heterocyclyl, —CO—C₂₋₂₀ heterocyclyl, or —C(O)NR₄-C₂₋₂₀ heterocyclyl; wherein, C₂₋₂₀ heterocyclyl, —CO—C₂₋₂₀ heterocyclyl, or —C(O)NR₄—C₂₋₂₀ heterocyclyl is optionally substituted with one or more groups selected from halogen, hydroxy, C₁₋₁₀ alkyl, —CH₂—NRₐC(O)R_{b}, —CRₐR_{b}—OR_{c}, —CRₐR_{b}—NR_{c}R_{d}, or —CH₂—NHC(O)NRₐR_{b}; wherein, Rₐ, R_{b}, R_{c}, and R_{d} are independently selected from hydrogen, C₁₋₁₀ alkyl, or —C(O)R″;
R₁ is selected from cyano or C₁₋₁₀ alkyl;
R₂ is selected from C₆₋₁₀ aryl, C₁₋₁₀ haloalkyl, C₇₋₁₆ alkylaryl, C₃₋₂₀ alkyl heteroaryl or C₃₋₂₀ alkyl heterocyclyl; wherein, C₆₋₁₀ aryl, C₁₋₁₀ haloalkyl, C₇₋₁₆ alkylaryl, C₃₋₂₀ alkyl heteroaryl, or C₃₋₂₀ alkyl heterocyclyl, is optionally substituted with one or more groups selected from cyano, hydroxy, —C(O)NH₂, or C₁₋₁₀ alkyl;
R₃ is selected from halogen, C₆₋₁₀ aryl, or C₂₋₁₀ heteroaryl; wherein, C₆₋₁₀ aryl, or C₂₋₁₀ heteroaryl, is optionally substituted with one or more groups selected from halogen, haloalkyl, hydroxy, amino, C₁₋₁₀ alkyl, OR″ or C₂₋₂₀ heterocyclyl;
R″ is selected from C₁₋₁₀ alkyl, or C₁₋₁₀ haloalkyl;
R₄ is hydrogen; and
n is 0 to 1,
provided that the compound of Formula (IA) is not:

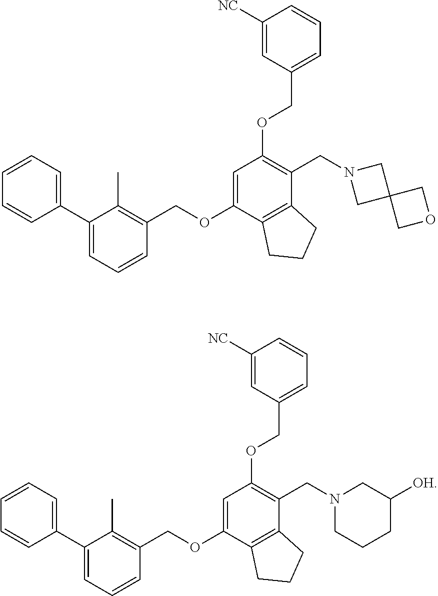 [SMILES: Cc1c(COc2cc(OCc3cccc(C#N)c3)c(CN3CC4(COC4)C3)c3c2CCC3)cccc1-c1ccccc1 ; Cc1c(COc2cc(OCc3cccc(C#N)c3)c(CN3CCCC(O)C3)c3c2CCC3)cccc1-c1ccccc1]

In an implementation of the present invention, there is provided a compound of Formula (IB),

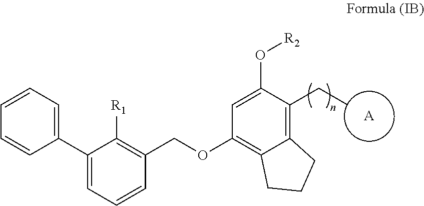

their stereoisomers, an N-oxide or pharmaceutically acceptable salts thereof;
wherein,
Ring A is selected from C₆₋₁₀ aryl, C₃₋₁₀ cycloalkyl, C₇₋₁₆ alkylaryl, C₂₋₁₀ heteroaryl, C₂₋₂₀ heterocyclyl, —CO—C₂₋₂₀ heterocyclyl, or —C(O)NR₄-C₂₋₂₀ heterocyclyl; wherein, C₆₋₁₀ aryl, C₃₋₁₀ cycloalkyl, C₇₋₁₆ alkylaryl, C₂₋₁₀ heteroaryl, C₂₋₂₀ heterocyclyl, —CO—C₂₋₂₀ heterocyclyl, or —C(O)NR₄—C₂₋₂₀ heterocyclyl is optionally substituted with one or more groups selected from halogen, hydroxy, C₁₋₁₀ alkyl, C₁₋₁₀ alkoxy, C₁₋₁₀ haloalkyl, C₂₋₁₀ alkylalkoxy, —CH₂—NRₐC(O)R_{b}, —CRₐR_{b}—OR_{c}, —CRₐR_{b}—NR_{c}R_{d}, or —CH₂—NHC(O)NRₐR_{b}; wherein, Rₐ, R_{b}, R_{c}, and R_{d} are independently selected from hydrogen, halogen, C₁₋₁₀ alkyl, —C(O)R″, C₃₋₁₀ cycloalkyl, C₁₋₁₀ haloalkyl, or C₁₋₁₀ alkoxy;
R₁ is selected from hydrogen, cyano or C₁₋₁₀ alkyl;
R₂ is selected from hydrogen, C₁₋₁₀ alkyl, C₆₋₁₀ aryl, C₃₋₁₀ cycloalkyl, C₁₋₁₀ haloalkyl, C₇₋₁₆ alkylaryl, C₂₋₁₀ heteroaryl, C₃₋₂₀ alkyl heteroaryl, C₂₋₂₀ heterocyclyl, or C₃₋₂₀ alkyl heterocyclyl; wherein, C₁₋₁₀ alkyl, C₆₋₁₀ aryl, C₃₋₁₀ cycloalkyl, C₁₋₁₀ haloalkyl, C₇₋₁₆ alkylaryl, C₂₋₁₀ heteroaryl, C₃₋₂₀ alkyl heteroaryl, C₂₋₂₀ heterocyclyl, or C₃₋₂₀ alkyl heterocyclyl, is optionally substituted with one or more groups selected from halogen, cyano, hydroxy, —C(O)NH₂, C₁₋₁₀ alkyl, or C₆₋₁₀ aryl;
R″ is selected from hydrogen, halogen, C₁₋₁₀ alkyl, or C₁₋₁₀ haloalkyl;
R₄ is selected from hydrogen or C₁₋₁₀ alkyl; and
n is 0 to 1.

In an implementation of the present invention, there is provided a compound of Formula (IC),

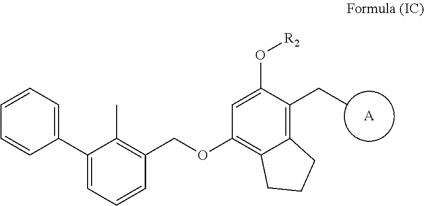

their stereoisomers, an N-oxide or pharmaceutically acceptable salts thereof;
wherein,
Ring A is selected from C₆₋₁₀ aryl, C₃₋₁₀ cycloalkyl, C₇₋₁₆ alkylaryl, C₂₋₁₀ heteroaryl, C₂₋₂₀ heterocyclyl, —CO—C₂₋₂₀ heterocyclyl, or —C(O)NR₄-C₂₋₂₀ heterocyclyl; wherein, C₆₋₁₀ aryl, C₃₋₁₀ cycloalkyl, C₇₋₁₆ alkylaryl, C₂₋₁₀ heteroaryl, C₂₋₂₀ heterocyclyl, —CO—C₂₋₂₀ heterocyclyl, or —C(O)NR₄—C₂₋₂₀ heterocyclyl is optionally substituted with one or more groups selected from halogen, hydroxy, C₁₋₁₀ alkyl, C₁₋₁₀ alkoxy, C₁₋₁₀ haloalkyl, C₂₋₁₀ alkylalkoxy, —CH₂—NRₐC(O)R_{b}, —CRₐR_{b}—OR_{c}, —CRₐR_{b}—NR_{c}R_{d}, or —CH₂—NHC(O)NRₐR_{b}; wherein, Rₐ, R_{b}, R_{c}, and R_{d} are independently selected from hydrogen, halogen, C₁₋₁₀ alkyl, —C(O)R″, C₃₋₁₀ cycloalkyl, C₁₋₁₀ haloalkyl, or C₁₋₁₀ alkoxy;
R₂ is selected from hydrogen, C₁₋₆ alkyl, C₆₋₁₀ aryl, C₃₋₁₀ cycloalkyl, C₁₋₆ haloalkyl, C₇₋₁₆ alkylaryl, C₂₋₁₀ heteroaryl, C₃₋₂₀ alkyl heteroaryl, C₂₋₂₀ heterocyclyl, or C₃₋₂₀ alkyl heterocyclyl; wherein, C₁₋₆ alkyl, C₆₋₁₀ aryl, C₃₋₁₀ cycloalkyl, C₁₋₆ haloalkyl, C₇₋₁₆ alkylaryl, C₂₋₁₀ heteroaryl, C₃₋₂₀ alkyl heteroaryl, C₂₋₂₀ heterocyclyl, or C₃₋₂₀ alkyl heterocyclyl, is optionally substituted with one or more groups selected from halogen, cyano, hydroxy, —C(O)NH₂, C₁₋₁₀ alkyl, or C₆₋₁₀ aryl;
R″ is selected from hydrogen, halogen, C₁₋₁₀ alkyl, or C₁₋₁₀ haloalkyl;
R₄ is selected from hydrogen or C₁₋₆ alkyl.

In an implementation of the present invention, there is provided a compound of Formula (IC), their stereoisomers, an N-oxide or pharmaceutically acceptable salts thereof;

wherein,
Ring A is selected from C₂₋₁₀ heterocyclyl; wherein, C₂₋₁₀ heterocyclyl is optionally substituted with one or more groups selected from halogen, hydroxy, C₁₋₆ alkyl, —CH₂—NRₐC(O)R_{b}, —CRₐR_{b}—OR_{c}, —CRₐR_{b}—NR_{c}R_{d}, or —CH₂—NHC(O)NRₐR_{b}; wherein, Rₐ, R_{b}, R_{c}, and R_{d} are independently selected from hydrogen or C₁₋₁₀ alkyl; and
R₂ is selected from C₆₋₁₀ aryl, C₁₋₆ haloalkyl, C₇₋₁₆ alkylaryl, C₃₋₂₀ alkyl heteroaryl, or C₂₋₂₀ alkyl heterocyclyl; wherein, C₆₋₁₀ aryl, C₁₋₆ haloalkyl, C₇₋₁₆ alkylaryl, C₃₋₂₀ alkyl heteroaryl, or C₂₋₂₀ alkyl heterocyclyl, is optionally substituted with one or more groups selected from cyano, hydroxy, —C(O)NH₂, or C₁₋₆ alkyl;
R″ is selected from hydrogen, halogen, C₁₋₁₀ alkyl, or C₁₋₁₀ haloalkyl; and
R₄ is selected from hydrogen or C₁₋₆ alkyl.

In an implementation of the present invention, there is provided compound of Formula (I) selected from:

| Example No. | IUPAC Name |
|---|---|
| 1 | (S)-5-(((4-((2-(hydroxymethyl)piperidin-1-yl)methyl)-7-((2-methyl- |
| | [1,1′-biphenyl]-3-yl)methoxy)-2,3-dihydro-1H-inden-5- |
| | yl)oxy)methyl)nicotinonitrile; |
| 2 | (S)-3-(((4-((2-(hydroxymethyl)piperidin-1-yl)methyl)-7-((2-methyl- |
| | [1,1′-biphenyl]-3-yl)methoxy)-2,3-dihydro-1H-inden-5- |
| | yl)oxy)methyl)benzonitrile; |
| 3 | (S)-5-(((7-((2-cyano-[1,1′-biphenyl]-3-yl)methoxy)-4-((2- |
| | (hydroxymethyl)pyrrolidin-1-yl)methyl)-2,3-dihydro-1H-inden-5- |
| | yl)oxy)methyl)nicotinonitrile; |
| 4 | (S)-3-(((6-((3-cyanobenzyl)oxy)-7-((2-(hydroxymethyl)pyrrolidin- |
| | 1-yl)methyl)-2,3-dihydro-1H-inden-4-yl)oxy)methyl)-[1,1′- |
| | biphenyl]-2-carbonitrile; |
| 5 | (S)-5-(((7-((2-cyano-[1,1′-biphenyl]-3-yl)methoxy)-4-((2- |
| | (hydroxymethyl)piperidin-1-yl)methyl)-2,3-dihydro-1H-inden-5- |
| | yl)oxy)methyl)nicotinonitrile; |
| 6 | (5-(((4-(((2S,4R)-4-hydroxy-2-(hydroxymethyl)pyrrolidin-1- |
| | yl)methyl)-7-((2-methyl-[1,1′-biphenyl]-3-yl)methoxy)-2,3-dihydro- |
| | 1H-inden-5-yl)oxy)methyl)nicotinonitrile; |
| 7 | (S)-3-(((4-((2-(hydroxymethyl)pyrrolidin-1-yl)methyl)-7-((2- |
| | methyl-[1,1′-biphenyl]-3-yl)methoxy)-2,3-dihydro-1H-inden-5- |
| | yl)oxy)methyl)benzonitrile; |
| 8 | (S)-5-(((4-((2-(hydroxymethyl)pyrrolidin-1-yl)methyl)-7-((2- |
| | methyl-[1,1′-biphenyl]-3-yl)methoxy)-2,3-dihydro-1H-inden-5- |
| | yl)oxy)methyl)nicotinonitrile; |
| 9 | (R)-5-(((4-((3-(hydroxymethyl)morpholino)methyl)-7-((2-methyl- |
| | [1,1′-biphenyl]-3-yl)methoxy)-2,3-dihydro-1H-inden-5- |
| | yl)oxy)methyl)nicotinonitrile; |
| 10 | 5-(((4-((2-(hydroxymethyl)azetidin-1-yl)methyl)-7-((2-methyl-[1,1′- |
| | biphenyl]-3-yl)methoxy)-2,3-dihydro-1H-inden-5- |
| | yl)oxy)methyl)nicotinonitrile; |
| 11 | 5-(((4-((6-(hydroxymethyl)-5-azaspiro[2.5]octan-5-yl)methyl)-7- |
| | ((2-methyl-[1,1′-biphenyl]-3-yl)methoxy)-2,3-dihydro-1H-inden-5- |
| | yl)oxy)methyl)nicotinonitrile; |
| 12 | 5-(((4-((5,5-difluoro-2-(hydroxymethyl)piperidin-1-yl)methyl)-7- |
| | ((2-methyl-[1,1′-biphenyl]-3-yl)methoxy)-2,3-dihydro-1H-inden-5- |
| | yl)oxy)methyl)nicotinonitrile; |
| 13 | 5-(((4-((2-(hydroxymethyl)-4-methylpiperazin-1-yl)methyl)-7-((2- |
| | methyl-[1,1′-biphenyl]-3-yl)methoxy)-2,3-dihydro-1H-inden-5- |
| | yl)oxy)methyl)nicotinonitrile; |
| 14 | 5-(((4-((3-(hydroxymethyl)azetidin-1-yl)methyl)-7-((2-methyl-[1,1′- |
| | biphenyl]-3-yl)methoxy)-2,3-dihydro-1H-inden-5- |
| | yl)oxy)methyl)nicotinonitrile; |
| 15 | (R)-5-(((4-((3-(hydroxymethyl)pyrrolidin-1-yl)methyl)-7-((2- |
| | methyl-[1,1′-biphenyl]-3-yl)methoxy)-2,3-dihydro-1H-inden-5- |
| | yl)oxy)methyl)nicotinonitrile; |
| 16 | (S)-5-(((4-((3-(hydroxymethyl)piperidin-1-yl)methyl)-7-((2-methyl- |
| | [1,1′-biphenyl]-3-yl)methoxy)-2,3-dihydro-1H-inden-5- |
| | yl)oxy)methyl)nicotinonitrile; |
| 17 | 5-(((4-((4-(hydroxymethyl)piperidin-1-yl)methyl)-7-((2-methyl- |
| | [1,1′-biphenyl]-3-yl)methoxy)-2,3-dihydro-1H-inden-5- |
| | yl)oxy)methyl)nicotinonitrile; |
| 18 | (S)-5-(((4-((3-(hydroxymethyl)pyrrolidin-1-yl)methyl)-7-((2- |
| | methyl-[1,1′-biphenyl]-3-yl)methoxy)-2,3-dihydro-1H-inden-5- |
| | yl)oxy)methyl)nicotinonitrile; |
| 19 | (R)-5-(((4-((2-(hydroxymethyl)pyrrolidin-1-yl)methyl)-7-((2- |
| | methyl-[1,1′-biphenyl]-3-yl)methoxy)-2,3-dihydro-1H-inden-5- |
| | yl)oxy)methyl)nicotinonitrile; |
| 20 | 5-(((4-((2-(hydroxymethyl)azepan-1-yl)methyl)-7-((2-methyl-[1,1′- |
| | biphenyl]-3-yl)methoxy)-2,3-dihydro-1H-inden-5- |
| | yl)oxy)methyl)nicotinonitrile; |
| 21 | (R)-5-(((4-((3-(hydroxymethyl)piperidin-1-yl)methyl)-7-((2-methyl- |
| | [1,1′-biphenyl]-3-yl)methoxy)-2,3-dihydro-1H-inden-5- |
| | yl)oxy)methyl)nicotinonitrile 2,2,2-trifluoroacetate; |
| 22 | 5-(((4-((2,2-bis(hydroxymethyl)piperidin-1-yl)methyl)-7-((2- |
| | methyl-[1,1′-biphenyl]-3-yl)methoxy)-2,3-dihydro-1H-inden-5- |
| | yl)oxy)methyl)nicotinonitrile 2,2,2-trifluoroacetate; |
| 23 | (S)-3-(((7-((2-(hydroxymethyl)piperidin-1-yl)methyl)-6-(2,2,2- |
| | trifluoroethoxy)-2,3-dihydro-1H-inden-4-yl)oxy)methyl)-[1,1′- |
| | biphenyl]-2-carbonitrile; |
| 24 | (S)-(1-((7-((2-methyl-[1,1′-biphenyl]-3-yl)methoxy)-5-(2,2,2- |
| | trifluoroethoxy)-2,3-dihydro-1H-inden-4-yl)methyl)piperidin-2- |
| | yl)methanol; |
| 25 | (S)-5-(((4-((6-(hydroxymethyl)-5-azaspiro[2.4]heptan-5-yl)methyl)- |
| | 7-((2-methyl-[1,1′-biphenyl]-3-yl)methoxy)-2,3-dihydro-1H-inden- |
| | 5-yl)oxy)methyl)nicotinonitrile; |
| 26 | (S)-5-(((7-((2-cyano-[1,1′-biphenyl]-3-yl)methoxy)-4-((6- |
| | (hydroxymethyl)-5-azaspiro[2.4]heptan-5-yl)methyl)-2,3-dihydro- |
| | 1H-inden-5-yl)oxy)methyl)nicotinonitrile; |
| 27 | (S)-(1-((5-(2-fluoroethoxy)-7-((2-methyl-[1,1′-biphenyl]-3- |
| | yl)methoxy)-2,3-dihydro-1H-inden-4-yl)methyl)piperidin-2- |
| | yl)methanol; |
| 28 | (S)-(1-((5-(2-fluoroethoxy)-7-((2-methyl-[1,1′-biphenyl]-3- |
| | yl)methoxy)-2,3-dihydro-1H-inden-4-yl)methyl)pyrrolidin-2- |
| | yl)methanol; |
| 29 | (S)-3-(((6-(2-fluoroethoxy)-7-((2-(hydroxymethyl)piperidin-1- |
| | yl)methyl)-2,3-dihydro-1H-inden-4-yl)oxy)methyl)-[1,1′-biphenyl]- |
| | 2-carbonitrile; |
| 30 | (S)-(1-((5-((5-fluoropentyl)oxy)-7-((2-methyl-[1,1′-biphenyl]-3- |
| | yl)methoxy)-2,3-dihydro-1H-inden-4-yl)methyl)piperidin-2- |
| | yl)methanol; |
| 31 | (S)-(1-((5-(4-fluorobutoxy)-7-((2-methyl-[1,1′-biphenyl]-3- |
| | yl)methoxy)-2,3-dihydro-1H-inden-4-yl)methyl)piperidin-2- |
| | yl)methanol; |
| 32 | (S)-(1-((5-(3-fluoropropoxy)-7-((2-methyl-[1,1′-biphenyl]-3- |
| | yl)methoxy)-2,3-dihydro-1H-inden-4-yl)methyl)piperidin-2- |
| | yl)methanol |
| 33 | (S)-(1-((5-(4-fluorobutoxy)-7-((2-methyl-[1,1′-biphenyl]-3- |
| | yl)methoxy)-2,3-dihydro-1H-inden-4-yl)methyl)azetidin-2- |
| | yl)methanol; |
| 34 | 3-(((4-((1-(hydroxymethyl)-2-azabicyclo[4.1.0]heptan-2-yl)methyl)- |
| | 7-((2-methyl-[1,1′-biphenyl]-3-yl)methoxy)-2,3-dihydro-1H-inden- |
| | 5-yl)oxy)methyl)benzonitrile; |
| 35 | 5-(((4-((1-(hydroxymethyl)-2-azabicyclo[4.1.0]heptan-2-yl)methyl)- |
| | 7-((2-methyl-[1,1′-biphenyl]-3-yl)methoxy)-2,3-dihydro-1H-inden- |
| | 5-yl)oxy)methyl)nicotinonitrile; |
| 36 | 3-(((6-((3-cyanobenzyl)oxy)-7-((1-(hydroxymethyl)-2- |
| | azabicyclo[4.1.0]heptan-2-yl)methyl)-2,3-dihydro-1H-inden-4- |
| | yl)oxy)methyl)-[1,1′-biphenyl]-2-carbonitrile; |
| 37 | (S)-5-(((4-((2-(hydroxymethyl)piperidin-1-yl)methyl)-7-((2-methyl- |
| | [1,1′-biphenyl]-3-yl)methoxy)-2,3-dihydro-1H-inden-5- |
| | yl)oxy)methyl)nicotinamide; |
| 38 | (S)-3-(((4-((2-(hydroxymethyl)pyrrolidin-1-yl)methyl)-7-((2- |
| | methyl-[1,1′-biphenyl]-3-yl)methoxy)-2,3-dihydro-1H-inden-5- |
| | yl)oxy)methyl)benzamide; |
| 39 | (S)-(1-((5-((1-methyl-1H-pyrazol-4-yl)methoxy)-7-((2-methyl-[1,1′- |
| | biphenyl]-3-yl)methoxy)-2,3-dihydro-1H-inden-4- |
| | yl)methyl)piperidin-2-yl)methanol; |
| 40 | (1-((7-((2-methyl-[1,1′-biphenyl]-3-yl)methoxy)-5-(thiazol-4- |
| | ylmethoxy)-2,3-dihydro-1H-inden-4-yl)methyl)azetidin-2- |
| | yl)methanol; |
| 41 | (1-((5-((1-methyl-1H-imidazol-4-yl)methoxy)-7-((2-methyl-[1,1′- |
| | biphenyl]-3-yl)methoxy)-2,3-dihydro-1H-inden-4- |
| | yl)methyl)azetidin-2-yl)methanol; |
| 42 | (1-((7-((2-methyl-[1,1′-biphenyl]-3-yl)methoxy)-5-(pyrimidin-5- |
| | ylmethoxy)-2,3-dihydro-1H-inden-4-yl)methyl)azetidin-2- |
| | yl)methanol; |
| 43 | (1-((7-((2-methyl-[1,1′-biphenyl]-3-yl)methoxy)-5-(oxazol-4- |
| | ylmethoxy)-2,3-dihydro-1H-inden-4-yl)methyl)azetidin-2- |
| | yl)methanol; |
| 44 | (S)-3-cyano-5-(((4-((2-(hydroxymethyl)pyrrolidin-1-yl)methyl)-7- |
| | ((2-methyl-[1,1′-biphenyl]-3-yl)methoxy)-2,3-dihydro-1H-inden-5- |
| | yl)oxy)methyl)pyridine 1-oxide; |
| 45 | (S)-3-cyano-5-(((4-((2-(hydroxymethyl)piperidin-1-yl)methyl)-7- |
| | ((2-methyl-[1,1′-biphenyl]-3-yl)methoxy)-2,3-dihydro-1H-inden-5- |
| | yl)oxy)methyl)pyridine 1-oxide; |
| 46 | (S)-5-(((7-((4′-fluoro-2-methyl-[1,1′-biphenyl]-3-yl)methoxy)-4-((2- |
| | (hydroxymethyl)piperidin-1-yl)methyl)-2,3-dihydro-1H-inden-5- |
| | yl)oxy)methyl)nicotinonitrile; |
| 47 | (S)-5-(((7-((4′-fluoro-2-methyl-[1,1′-biphenyl]-3-yl)methoxy)-4-((6- |
| | (hydroxymethyl)-5-azaspiro[2.4]heptan-5-yl)methyl)-2,3-dihydro- |
| | 1H-inden-5-yl)oxy)methyl)nicotinonitrile; |
| 48 | (S)-5-(((7-((4′-fluoro-2-methyl-[1,1′-biphenyl]-3-yl)methoxy)-4-((2- |
| | (hydroxymethyl)pyrrolidin-1-yl)methyl)-2,3-dihydro-1H-inden-5- |
| | yl)oxy)methyl)nicotinonitrile; |
| 49 | 5-((5-cyanopyridin-3-yl)methoxy)-7-((2-methyl-[1,1′-biphenyl]-3- |
| | yl)methoxy)-N-(1-methylpiperidin-4-yl)-2,3-dihydro-1H-indene-4- |
| | carboxamide; |
| 50 | (S)-5-(((4-(2-(hydroxymethyl)pyrrolidine-1-carbonyl)-7-((2-methyl- |
| | [1,1′-biphenyl]-3-yl)methoxy)-2,3-dihydro-1H-inden-5- |
| | yl)oxy)methyl)nicotinonitrile; |
| 51 | (S)-5-(((4-(3-(hydroxymethyl)pyrrolidine-1-carbonyl)-7-((2-methyl- |
| | [1,1′-biphenyl]-3-yl)methoxy)-2,3-dihydro-1H-inden-5- |
| | yl)oxy)methyl)nicotinonitrile; |
| 52 | N-((1-((5-((5-cyanopyridin-3-yl)methoxy)-7-((2-methyl-[1,1′- |
| | biphenyl]-3-yl)methoxy)-2,3-dihydro-1H-inden-4- |
| | yl)methyl)pyrrolidin-2-yl)methyl)acetamide; |
| 53 | 5-(((4-((2-(aminomethyl)pyrrolidin-1-yl)methyl)-7-((2-methyl-[1,1′- |
| | biphenyl]-3-yl)methoxy)-2,3-dihydro-1H-inden-5- |
| | yl)oxy)methyl)nicotinonitrile; |
| 54 | 5-(((7-((3-bromo-2-methylbenzyl)oxy)-4-((2- |
| | (hydroxymethyl)azetidin-1-yl)methyl)-2,3-dihydro-1H-inden-5- |
| | yl)oxy)methyl)nicotinonitrile; |
| 55 | 5-(((7-((4′-hydroxy-2-methyl-[1,1′-biphenyl]-3-yl)methoxy)-4-((2- |
| | (hydroxymethyl)azetidin-1-yl)methyl)-2,3-dihydro-1H-inden-5- |
| | yl)oxy)methyl)nicotinonitrile; |
| 56 | 5-(((7-((2′-fluoro-2,5′-dimethyl-[1,1′-biphenyl]-3-yl)methoxy)-4- |
| | ((2-(hydroxymethyl)azetidin-1-yl)methyl)-2,3-dihydro-1H-inden-5- |
| | yl)oxy)methyl)nicotinonitrile; |
| 57 | 5-(((4-((2-(hydroxymethyl)azetidin-1-yl)methyl)-7-((2-methyl-3- |
| | (1H-pyrazol-4-yl)benzyl)oxy)-2,3-dihydro-1H-inden-5- |
| | yl)oxy)methyl)nicotinonitrile; |
| 58 | 5-(((4-((2-(hydroxymethyl)azetidin-1-yl)methyl)-7-((4′-methoxy- |
| | 2,2′-dimethyl-[1,1′-biphenyl]-3-yl)methoxy)-2,3-dihydro-1H-inden- |
| | 5-yl)oxy)methyl)nicotinonitrile; |
| 59 | 5-(((4-((2-(hydroxymethyl)azetidin-1-yl)methyl)-7-((2-methyl-3- |
| | (pyrimidin-5-yl)benzyl)oxy)-2,3-dihydro-1H-inden-5- |
| | yl)oxy)methyl)nicotinonitrile; |
| 60 | 5-(((4-((2-(hydroxymethyl)azetidin-1-yl)methyl)-7-((2-methyl-2′- |
| | (trifluoromethyl)-[1,1′-biphenyl]-3-yl)methoxy)-2,3-dihydro-1H- |
| | inden-5-yl)oxy)methyl)nicotinonitrile; |
| 61 | 5-(((4-((2-(hydroxymethyl)azetidin-1-yl)methyl)-7-((2-methyl-3-(6- |
| | morpholinopyridin-3-yl)benzyl)oxy)-2,3-dihydro-1H-inden-5- |
| | yl)oxy)methyl)nicotinonitrile; |
| 62 | 5-(((7-((2′-fluoro-2-methyl-[1,1′-biphenyl]-3-yl)methoxy)-4-((2- |
| | (hydroxymethyl)azetidin-1-yl)methyl)-2,3-dihydro-1H-inden-5- |
| | yl)oxy)methyl)nicotinonitrile; |
| 63 | 5-(((4-((2-(hydroxymethyl)azetidin-1-yl)methyl)-7-((4′-methoxy-2- |
| | methyl-[1,1′-biphenyl]-3-yl)methoxy)-2,3-dihydro-1H-inden-5- |
| | yl)oxy)methyl)nicotinonitrile; |
| 64 | 5-(((7-((3′-fluoro-2-methyl-[1,1′-biphenyl]-3-yl)methoxy)-4-((2- |
| | (hydroxymethyl)azetidin-1-yl)methyl)-2,3-dihydro-1H-inden-5- |
| | yl)oxy)methyl)nicotinonitrile; |
| 65 | 5-(((4-((2-(hydroxymethyl)azetidin-1-yl)methyl)-7-((3′-methoxy-2- |
| | methyl-[1,1′-biphenyl]-3-yl)methoxy)-2,3-dihydro-1H-inden-5- |
| | yl)oxy)methyl)nicotinonitrile; |
| 66 | 5-(((4-((2-(hydroxymethyl)azetidin-1-yl)methyl)-7-((2-methyl-4′- |
| | (trifluoromethoxy)-[1,1′-biphenyl]-3-yl)methoxy)-2,3-dihydro-1H- |
| | inden-5-yl)oxy)methyl)nicotinonitrile; |
| 67 | 5-(((7-((3′-hydroxy-2-methyl-[1,1′-biphenyl]-3-yl)methoxy)-4-((2- |
| | (hydroxymethyl)azetidin-1-yl)methyl)-2,3-dihydro-1H-inden-5- |
| | yl)oxy)methyl)nicotinonitrile; |
| 68 | 5-(((4-((2-(hydroxymethyl)azetidin-1-yl)methyl)-7-((2-methyl-3-(1- |
| | methyl-1H-pyrazol-4-yl)benzyl)oxy)-2,3-dihydro-1H-inden-5- |
| | yl)oxy)methyl)nicotinonitrile; |
| 69 | 5-(((4-((2-(hydroxymethyl)azetidin-1-yl)methyl)-7-((2-methyl-3- |
| | (quinolin-8-yl)benzyl)oxy)-2,3-dihydro-1H-inden-5- |
| | yl)oxy)methyl)nicotinonitrile; |
| 70 | 5-(((4-((2-(hydroxymethyl)azetidin-1-yl)methyl)-7-((2-methyl-3- |
| | (pyridin-4-yl)benzyl)oxy)-2,3-dihydro-1H-inden-5- |
| | yl)oxy)methyl)nicotinonitrile; |
| 71 | 5-(((4-((2-(hydroxymethyl)azetidin-1-yl)methyl)-7-((2-methyl-3- |
| | (pyridin-3-yl)benzyl)oxy)-2,3-dihydro-1H-inden-5- |
| | yl)oxy)methyl)nicotinonitrile; |
| 72 | (S)-5-(((4-((2-(hydroxymethyl)azetidin-1-yl)methyl)-7-((2-methyl- |
| | 3-(1H-pyrrolo[2,3-b]pyridin-5-yl)benzyl)oxy)-2,3-dihydro-1H- |
| | inden-5-yl)oxy)methyl)nicotinonitrile; |
| 73 | 5-(((7-((2,4′-dimethyl-[1,1′-biphenyl]-3-yl)methoxy)-4-((2- |
| | (hydroxymethyl)azetidin-1-yl)methyl)-2,3-dihydro-1H-inden-5- |
| | yl)oxy)methyl)nicotinonitrile; |
| 74 | 5-(((4-((2-(hydroxymethyl)azetidin-1-yl)methyl)-7-((2-methyl-3- |
| | (1H-pyrrolo[2,3-b]pyridin-5-yl)benzyl)oxy)-2,3-dihydro-1H-inden- |
| | 5-yl)oxy)methyl)nicotinonitrile 2,2,2-trifluoroacetate; |
| 75 | 5-(((7-((4′-(tert-butyl)-2-methyl-[1,1′-biphenyl]-3-yl)methoxy)-4- |
| | ((2-(hydroxymethyl)azetidin-1-yl)methyl)-2,3-dihydro-1H-inden-5- |
| | yl)oxy)methyl)nicotinonitrile; |
| 76 | 5-(((7-((4′-fluoro-2-methyl-[1,1′-biphenyl]-3-yl)methoxy)-4-((2- |
| | (hydroxymethyl)azetidin-1-yl)methyl)-2,3-dihydro-1H-inden-5- |
| | yl)oxy)methyl)nicotinonitrile; |
| 77 | 5-(((7-((3-(6-aminopyridin-3-yl)-2-methylbenzyl)oxy)-4-((2- |
| | (hydroxymethyl)azetidin-1-yl)methyl)-2,3-dihydro-1H-inden-5- |
| | yl)oxy)methyl)nicotinonitrile; |
| 77A | (S)-5-(((7-((3-(6-aminopyridin-3-yl)-2-methylbenzyl)oxy)-4-((2- |
| | (hydroxymethyl)azetidin-1-yl)methyl)-2,3-dihydro-1H-inden-5- |
| | yl)oxy)methyl)nicotinonitrile; (Isomer 1 of example 77) |
| 78 | (1-((7-((2-methyl-[1,1′-biphenyl]-3-yl)methoxy)-5-phenoxy-2,3- |
| | dihydro-1H-inden-4-yl)methyl)azetidin-2-yl)methanol; |
| 79 | (1-((7-((2-methyl-[1,1′-biphenyl]-3-yl)methoxy)-5-(pyrazin-2- |
| | ylmethoxy)-2,3-dihydro-1H-inden-4-yl)methyl)azetidin-2- |
| | yl)methanol. |
| 80 | 1-((1-((5-((5-cyanopyridin-3-yl)methoxy)-7-((2-methyl-[1,1′- |
| | biphenyl]-3-yl)methoxy)-2,3-dihydro-1H-inden-4- |
| | yl)methyl)pyrrolidin-2-yl)methyl)-3-methylurea formate; and |
| 81 | (1-((7-((2-methyl-[1,1′-biphenyl]-3-yl)methoxy)-5-((1- |
| | methylpiperidin-4-yl)methoxy)-2,3-dihydro-1H-inden-4- |
| | yl)methyl)azetidin-2-yl)methanol. |

or stereoisomers thereof, a pharmaceutically acceptable salts thereof, or an N-oxide thereof.

In an implementation of the present invention, there is provided compound of Formula (I), their stereoisomers, an N-oxide or pharmaceutically acceptable salts thereof, wherein the compound acts as inhibitors for PD1/PD-L1 interaction.

In an implementation of the present invention, there is provided a process for preparation of compounds of Formula (I), their stereoisomers, an N-oxide or pharmaceutically acceptable salts thereof, comprising the steps of: (a) reacting compounds of Formula (Ia) with a compound A in the presence of a reducing agent and a solvent to obtain compounds of Formula (I):

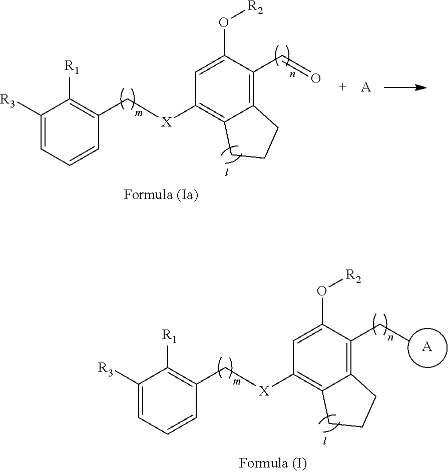

In an implementation of the present invention, there is provided a process for preparation of compounds of Formula (I) as disclosed herein, wherein the process is carried out at a temperature in the range of 25 to 80° C. for a time period in the range of 2 hours to 20 hours; the reducing agent is selected from sodium cyanoborohydride, sodium triacetoxyborohydride or sodium borohydride and the solvent is selected from methanol, ethanol, dimethyl formamide or combinations thereof.

In an implementation of the present invention, there is provided a process of preparation of compounds of Formula (I) as disclosed herein, wherein the Formula I optionally reacted with potassium tertiary butoxide in the presence of a solvent selected from tetrahydrofuran, t-butanol or combinations thereof.

In an implementation of the present invention, there is provided a process for preparation of compounds of Formula (I), their stereoisomers, an N-oxide or pharmaceutically acceptable salts thereof, comprising the steps of: (a) reacting compounds of Formula (Ia) with a compound A in the presence of sodium cyanoborohydride or sodium triacetoxyborohydride or sodium borohydride and a solvent selected from methanol, ethanol, dimethyl formamide or combinations thereof at a temperature in the range of 25 to 80° C. for a time period in the range of 2 to 20 hours to obtain compounds of Formula I.

In an implementation of the present invention, there is provided a process for preparation of compounds of Formula (I), their stereoisomers, an N-oxide or pharmaceutically acceptable salts thereof, comprising the steps of: (a) reacting compounds of Formula (Ia) with a compound A in the presence of sodium cyanoborohydride or sodium triacetoxyborohydride or sodium borohydride and a solvent selected from methanol, ethanol, dimethyl formamide or combinations thereof at a temperature in the range of 25 to 80° C. for a time period in the range of 2 hours to 20 hours to obtain compounds of Formula (I) and wherein the Formula (I) further reacted with potassium tertiary butoxide in the presence of a solvent selected from tetrahydrofuran, t-butanol or combinations thereof.

In an implementation of the present invention, there is provided a pharmaceutical composition comprising a compound of Formula (I) or a pharmaceutically acceptable salt thereof, together with a pharmaceutically acceptable carrier, optionally in combination with one or more other pharmaceutical compositions.

In an implementation of the present invention, there is provided a pharmaceutical composition comprising a compound of Formula (I) or a pharmaceutically acceptable salt thereof together with a pharmaceutically acceptable carrier, optionally in combination with one or more other pharmaceutical compositions, wherein the composition is in the form selected from the group consisting of a tablet, capsule, powder, syrup, solution, aerosol and suspension.

In an implementation of the present invention, there is provided a method for the treatment and/or prevention of a condition mediated by PD-1/PD-L1 or a proliferative disorder or cancer, comprising administering to a subject suffering from the condition mediated by PD-1/PD-L1 or the proliferative disorder or cancer, a therapeutically effective amount of the compounds of Formula (I) or the pharmaceutical composition as disclosed herein.

In an implementation of the present invention, there is provided a compounds of Formula (I) or the pharmaceutical composition as disclosed herein, for use in the manufacture of a medicament for inhibiting PD-1/PD-L1 interaction in a cell.

In an implementation of the present invention, there is provided a compound of Formula (I) or the pharmaceutical composition as disclosed herein, for use in the treatment and/or prevention of a condition mediated by PD-1/PD-L1 interaction or a proliferative disorder or cancer, comprising administering to a subject suffering from the condition mediated by PD-1/PD-L1 interaction or the proliferative disorder or cancer.

In an implementation of the present invention, there is provided a method for the treatment or prevention of disease or proliferative disorder or cancer comprising administering to a subject suffering from the disease or proliferative disorder or cancer a therapeutically effective amount of the compound of Formula (I) or the pharmaceutical composition as disclosed herein, with other clinically relevant cytotoxic agents or non-cytotoxic agents to a subject in need thereof.

In an implementation of the present invention, there is provided a method for the treatment or prevention of diseases, cancer or infectious diseases selected from metastatic cancer, breast cancer, prostate cancer, pancreatic cancer, gastric cancer, lung cancer, colon cancer, rectal cancer, esophagus cancer, duodenal cancer, tongue cancer, pharyngeal cancer, brain tumor, neurinoma, clear cell carcinoma, non-small cell lung cancer, small cell lung cancer, liver cancer, kidney cancer, Hodgkin's lymphoma, head and neck cancer, urothelial cancer, bile duct cancer, uterine body cancer, cervical cancer, ovarian cancer, urinary bladder, skin cancer, hemangioma, malignant lymphoma, malignant melanoma, thyroid cancer, bone tumor, vascular fibroma, glioblastoma, neuroblastoma, hepatoblastoma, medulloblastoma, nephroblastoma, pancreatoblastoma, pleuropulmonary blastoma, sarcoma, neuroendocrine tumors, retinoblastoma, penile cancer, pediatric solid cancer, renal cell carcinoma, lymphoma, myeloma, leukemia, acute myelogenous leukemia (AML), chronic myelogenous leukemia (CML), chronic neutrophilic leukemia, chronic eosinophilic leukemia, chronic lymphocytic leukemia (CLL), acute lymphoblastic leukemia (ALL), hairy cell leukemia, cutaneous T-cell lymphoma (CTCL), multiple myeloma (MM), metastatic cancer, Myeloproliferative neoplasms (MPN), a disease category that includes polycythemia vera (PV), essential thrombocythemia, essential thrombocytosis (ET) and myelofibrosis (MF), chronic myelogenous leukemia (CML), chronic neutrophilic leukemia (CNL), chronic eosinophilic leukemia (CEL), cancers with mutations in specific oncogenes, EGFR, KRAS, or RET comprising administering to a subject suffering from the proliferative disorder or cancer a therapeutically effective amount of the compound of Formula (I) or the pharmaceutical composition as disclosed herein, with other clinically relevant cytotoxic agents or non-cytotoxic agents to a subject in need thereof.

In an implementation of the present invention, there is provided use of the compound of Formula (I) or the pharmaceutical composition as disclosed herein, for the treatment or prevention of various diseases including proliferative disorder or cancer; or treatment of cancer together with other clinically relevant cytotoxic agents or non-cytotoxic agents.

In an implementation of the present invention, there is provided a method for the treatment of cancer, said method comprising administering a combination of the compounds of Formula (I) or the pharmaceutical composition as disclosed herein, with other clinically relevant cytotoxic agents or non-cytotoxic agents to a subject in need thereof.

In an implementation of the present invention, there is provided a method of treatment of cancer, said method comprising administering a combination of the compounds of Formula (I), or the pharmaceutical composition as disclosed herein with other clinically relevant immune modulators agents to a subject in need of thereof.

In an implementation of the present invention, there is provided a method of treating and/or preventing a disease or disorder comprising administering, to a patient in need of treatment, a therapeutically effective amount of a composition comprising a compound of Formula (I) and a pharmaceutically acceptable carrier.

In an implementation of the present invention, there is provided a compound of Formula (I) for use in treating and/or preventing a disease, a disorder or condition. In a related aspect, the invention provides for the use of a compound of Formula (I) for the manufacture of a medicament for treating and/or preventing a disease, disorder or condition.

In an implementation of the present invention, there is provided a method for the treatment or prevention of metastatic cancer selected from brain metastasis, bladder metastasis, breast metastasis, colon metastasis, kidney metastasis, lung metastasis, melanoma metastasis, ovary metastasis, pancreas metastasis, prostate metastasis, rectal metastasis, stomach metastasis, thyroid metastasis, or uterus metastasis, said method comprising administering a combination of the compounds of Formula (I), or the pharmaceutical composition as disclosed herein with other clinically relevant immune modulators agents to a subject in need of thereof.

In an implementation of the present invention, there is provided a compound of Formula (I) or the pharmaceutical composition as disclosed herein, wherein the compound of Formula (I) or the pharmaceutical composition acts as inhibitors for PD-1/PD-L1 interactions for brain metastasis.

In an implementation of the present disclosure, there is provided a compound of Formula (I) or the pharmaceutical composition as therapy for brain metastasis and for reducing neurologic toxicity risks associated with radiotherapy or radionecrosis.

In an implementation of the present invention, there is provided a compound which may be administered in combination therapy. “Combination therapy” includes the administration of the subject compounds in further combination with other biologically active ingredients (such as, but are not limited to, different antineoplastic agent) and non-drug therapies (such as, but are not limited to, surgery or radiation treatment). The compounds described herein can be used in combination with other pharmaceutically active compounds, preferably, which will enhance the effect of the compounds of the invention. The compounds can be administered simultaneously or sequentially to the other drug therapy.

In an implementation of the present invention, the subject compounds may be combined with the antineoplastic agents (e.g. small molecules, cytotoxic reagents, non-cytotoxic reagents, monoclonal antibodies, antisense RNA and fusion proteins) that inhibit one or more biological targets. Such combination may enhance therapeutic efficacy over the efficacy achieved by any of the agents alone and may prevent or delay the appearance of resistant variants.

### EXAMPLES

The following examples provide details about the synthesis, activities, and applications of the compounds of the present invention. It should be understood the following is representative only, and that the invention is not limited by the details set forth in these examples.

There is also provided a process as shown in the following scheme-1, for the preparation of compounds of the Formula (I), wherein all the groups are as defined earlier. The intermediate aldehydes used for the synthesis were prepared according to the method mentioned in WO2019/175897.

The following abbreviations refer respectively to the definitions herein: rt (Retention time); RT (Room temperature); ° C. (degree Celsius); DMF (Dimethyl formamide); h (hour); THF (tetrahydrofuran); HCl (Hydrochloric acid); DCM, CH₂Cl₂ (Dichloromethane); TFA (Trifluoroacetic acid); TLC (Thin layer chromatography); Na₂SO₄ (Sodium sulphate); ACN/CH₃CN (acetonitrile); AcOH (Acetic acid); MeOH (Methanol); DMSO-d₆ (Dimethyl sulfoxide-d); HPLC (High pressure liquid chromatography); LCMS (Liquid chromatography mass spectrometry); NMR (Nuclear magnetic resonance); TEA (triethyl amine); Cs₂CO₃ (Cesium carbonate); BH₃-DMS (Borane-DMS); K₂CO₃ (Potassium carbonate); MHz (megahertz); s (singlet); m (multiplet); and d (doublet). NMM (N-Methylmorpholine); KO^{t}Bu (potassium tert butoxide); t-BuOH (tert butyl alcohol); LAF (Lithium aluminum hydride); LAH (Lithium aluminium hydride); MsCl (methanesulphonyl chloride); mCPBA (3-chlorobenzene-1-carboperoxoic acid/meta-Chloroperoxybenzoic acid); Et₃N (triethylamine); Na(CN)BH₃/NaBH₃CN (Sodium cyanoborohydride); PPh₃ (triphenylphosphane); Pd(dppf)Cl₂ ([1,1′-Bis(diphenylphosphino)ferrocene]dichloropalladium(II)); PdCl₂(PPh₃)₂ (Bis(triphenylphosphine)palladium(II) dichloride); LiOH (Lithium hydroxide); NaBH₄ (Sodium borohydride); PBr₃ (tribromophosphane); POBr₃ (Tribromo phosphane/Phosphoryl bromide); HATU (1-[Bis(dimethylamino)methylene]-1H-1,2,3-triazolo[4,5-b]pyridinium 3-oxide hexafluorophosphate/Hexafluorophosphate azabenzotriazole tetramethyl uronium); DIPEA (N,N-Diisopropylethylamine).

### EXAMPLES

The present invention is further exemplified, but not limited, by following examples that illustrate the preparation of compounds according to the invention.

### Example 1: Synthesis of (S)-5-(((4-((2-(hydroxymethyl)piperidin-1-yl)methyl)-7-((2-methyl-[1,1′-biphenyl]-3-yl)methoxy)-2,3-dihydro-1H-inden-5-yl)oxy)methyl)nicotinonitrile

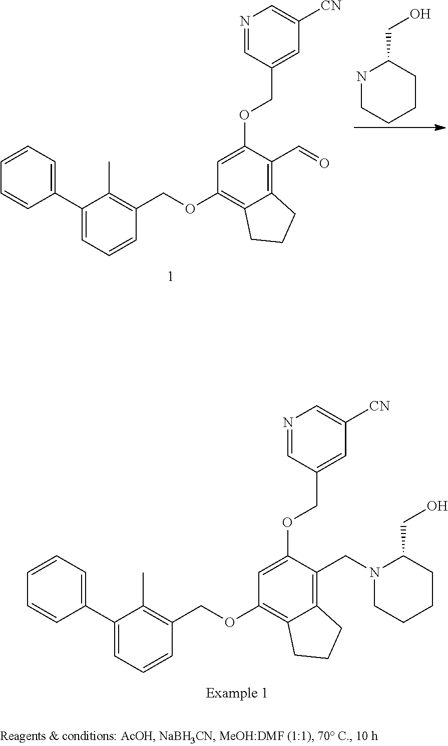 [SMILES: Cc1c(COc2cc(OCc3cncc(C#N)c3)c(C=O)c3c2CCC3)cccc1-c1ccccc1 ; OC[C@@H]1CCCCN1 ; Cc1c(COc2cc(OCc3cncc(C#N)c3)c(CN3CCCC[C@H]3CO)c3c2CCC3)cccc1-c1ccccc1]

A solution of 5-(((4-formyl-7-((2-methyl-[1,1′-biphenyl]-3-yl)methoxy)-2,3-dihydro-1H-inden-5-yl)oxy)methyl)nicotinonitrile (1, 0.3 g, 0.63 mmol), (S)-piperidin-2-ylmethanol (0.076 g, 0.76 mmol), sodium cyanoborohydride (0.118 g, 0.18 mmol) and acetic acid (2 drops) in methanol (5 mL) and N,N-dimethylformamide (5 mL) was heated at 70° C. for 10 h. After completion of the reaction, the reaction mixture was diluted with water (10 mL) and extracted with 10% methanol in dichloromethane (3×35 mL). The organic layer was dried over anhydrous sodium sulfate and concentrated. The resulting crude was purified by silica gel flash column chromatography using 10% methanol in dichloromethane as eluent to get the desired compound. The compound was again purified by reverse phase prep-HPLC (ammonium acetate buffer) to afford the title product (Example 1, 0.15 g, 41%) as white solid.

LCMS (ES) m/z=574.43 [M+H]⁺; ¹H NMR (400 MHz, DMSO-d₆) δ ppm: 1.66 (m, 4H), 1.98 (m, 2H), 2.21 (s, 3H), 2.60 (m, 1H), 2.49-2.86 (m, 5H), 3.16 (m, 1H), 3.40 (m, 1H), 3.68 (m, 1H), 3.96-4.02 (m, 2H), 4.28 (m, 1H), 4.65 (m, 1H), 5.13-5.40 (m, 5H), 6.74-6.88 (bs, 1H), 7.13 (m, 1H), 7.20-7.31 (m, 3H), 7.39 (m, 1H), 7.44-7.48 (m, 3H), 8.49 (m, 1H), 9.02 (in, 2H). HPLC: 98.54%

The compounds listed in below Table-1 were prepared by a procedure similar to the one described in Example-1 with appropriate variations in reactants, quantity of reagents, protections & deprotections, solvents & reaction conditions. The characterization data of the compounds are summarized herein below table.

TABLE 1

| Example | | |
|---|---|---|
| No. | Structure | Characterization data |
| 2 | 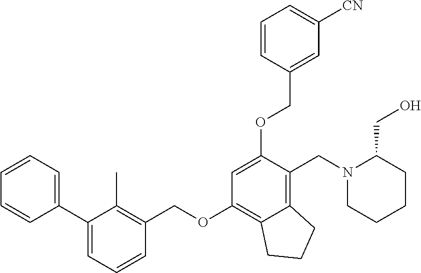 [SMILES: Cc1c(COc2cc(OCc3cccc(C#N)c3)c(CN3CCCC[C@H]3CO)c3c2CCC3)cccc1-c1ccccc1] | LCMS (ES) m/z = 573.48 [M + H]⁺; ¹H NMR (400 MHz, DMSO-d₆) δ ppm: 1.20-1.35 (m, 3H), 1.36-1.42 (m, 1H), 1.56-1.70 (m, 2H), 1.90- 2.00 (m, 3H), 2.20 (s, 3H), 2.21- 2.25 (m, 1H), 2.53-2.62 (m, 1H), 2.70-3.00 (m, 4H), 3.22 (d, J = 12.0 Hz, 1H), 3.40-3.45 (m, 1H), 3.68- 3.75 (m, 1H), 3.97 (d, J = 12.0 Hz, 1H), 4.30 (bs, 1H), 5.11 (s, 2H), 5.18-5.26 (m, 2H), 6.70 (s, 1H), 7.19 (d, J = 7.6 Hz, 1H), 7.28 (t, J = 7.2 Hz, 1H), 7.30-7.47 (m, 6H), 7.62 (t, J = 8.0 Hz, 1H), 7.80 (d, J = 7.6 Hz, 1H), 7.85 (d, J = 8.0 Hz, 1H), 7.95 (s, 1H); HPLC: 98.85%. |
| 3 | 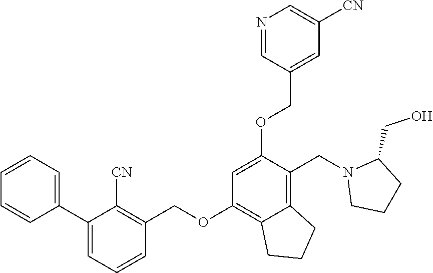 [SMILES: N#Cc1cncc(COc2cc(OCc3cccc(-c4ccccc4)c3C#N)c3c(c2CN2CCC[C@H]2CO)CCC3)c1] | LCMS (ES) m/z = 571.40 [M + H]⁺; ¹H NMR (400 MHz, DMSO-d₆) δ ppm: 1.48-1.60 (m, 3H), 1.75-1.85 (m, 1H), 1.92-2.02 (m, 2H), 2.12- 2.22 (m, 1H), 2.62-2.70 (m, 1H), 2.73-3.00 (m, 4H), 3.12-3.20 (m, 1H), 3.36-3.47 (m, 3H), 3.80-3.86 (m, 1H), 4.21 (bs, 1H), 5.21-5.38 (m, 4H), 6.73 (s, 1H), 7.49-7.64 (m, 6H), 7.72 (d, J = 7.6 Hz, 1H), 7.77 (t, J = 7.6 Hz, 1H), 8.42 (s, 1H), 8.98 (s, 2H); HPLC: 98.37%. |
| 4 | 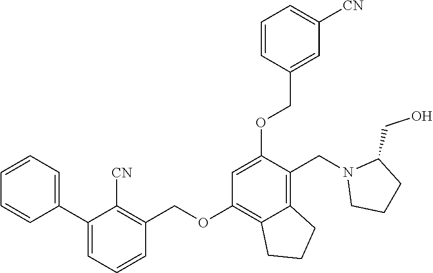 [SMILES: N#Cc1cccc(COc2cc(OCc3cccc(-c4ccccc4)c3C#N)c3c(c2CN2CCC[C@H]2CO)CCC3)c1] | LCMS (ES) m/z = 570.37 [M + H]⁺; ¹H NMR (400 MHz, DMSO-d₆) δ ppm: 1.52-1.58 (m, 3H), 1.81-1.84 (m, 1H), 1.96-2.01 (m, 2H), 2.15- 2.22 (m, 1H), 2.73-2.78 (m, 2H), 2.83-2.97 (m, 2H), 3.15-3.23 (m, 1H), 3.38-3.44 (m, 2H), 3.82 (d, J = 11.6 Hz, 1H), 4.20-4.23 (bs, 1H), 5.16-5.26 (m, 2H), 5.30 (s, 2H), 6.69 (s, 1H), 7.49-7.63 (m, 7H), 7.70 (d, J = 7.6 Hz, 1H), 7.76- 7.82 (m, 3H), 7.93 (s, 1H); 2H merged with DMSO residual peak. HPLC: 99.48%. |
| 5 | 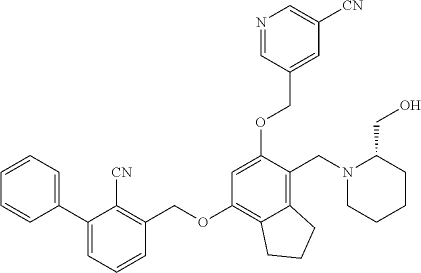 [SMILES: N#Cc1cncc(COc2cc(OCc3cccc(-c4ccccc4)c3C#N)c3c(c2CN2CCCC[C@H]2CO)CCC3)c1] | LCMS (ES) m/z = 585.23 [M + H]⁺; ¹H NMR (400 MHz, DMSO-d₆) δ ppm: 1.24-1.42 (m, 4H), 1.58-1.70 (m, 2H), 1.95-2.05 (m, 2H), 2.25- 2.32 (m, 1H), 2.55-2.62 (m, 2H), 2.78-2.84 (m, 2H), 2.86-3.00 (m, 2H), 3.24 (d, J = 8.0 Hz, 1H), 3.41- 3.43 (m, 1H), 3.65-3.72 (m, 1H), 3.95-4.00 (m, 2H), 5.20-5.30 (m, 2H), 5.31 (s, 2H), 6.71 (s, 1H), 7.49-7.61 (m, 6H), 7.72 (d, J = 7.6 Hz, 1H), 7.75-7.80 (m, 1H), 8.32 (s, 1H), 8.94 (s, 1H), 8.97 (d, J = 2.0 Hz, 1H). HPLC: 95.72%. |
| 6 | 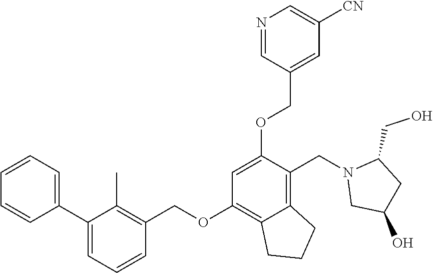 [SMILES: Cc1c(COc2cc(OCc3cncc(C#N)c3)c(CN3C[C@H](O)C[C@H]3CO)c3c2CCC3)cccc1-c1ccccc1] | LCMS (ES) m/z = 576.43 [M + H]⁺; ¹H NMR (400 MHz, DMSO-d₆) δ ppm: 1.60-1.78 (m, 2H), 1.92-2.02 (m, 2H), 2.10-2.18 (m, 1H), 2.20 (s, 3H), 2.70-3.00 (m, 6H), 3.10-3.20 (m, 1H), 3.40-3.46 (m, 2H), 3.80- 3.85 (m, 1H), 3.95-4.05 (m, 1H), 4.18 (bs, 1H), 4.61 (bs, 1H), 5.10- 5.16 (m, 2H), 5.20-5.32 (m, 2H), 6.74 (s, 1H), 7.19 (d, J = 6.8 Hz, 1H), 7.24-7.40 (m, 4H), 7.40-7.48 (m, 3H), 8.43 (s, 1H), 8.98 (s, 2H), HPLC: 96.95%. |
| 7 | 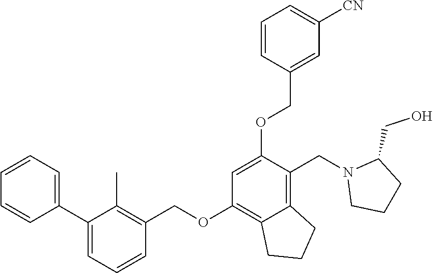 [SMILES: Cc1c(COc2cc(OCc3cccc(C#N)c3)c(CN3CCC[C@H]3CO)c3c2CCC3)cccc1-c1ccccc1] | LCMS (ES) m/z = 559.4 [M + H]⁺; ¹H NMR (400 MHz, DMSO-d₆) δ ppm: 1.50-1.60 (m, 3H), 1.75-1.85 (m, 1H), 1.90-2.00 (m, 2H), 2.20 (s, 4H), 2.66-2.75 (m, 3H), 2.80-3.00 (m, 2H), 3.15-3.23 (m, 1H), 3.40- 3.47 (m, 2H), 3.76-3.74 (m, 1H), 4.20 (bs, 1H), 5.11 (m, 2H), 5.15- 5.25 (m, 2H), 6.70 (s, 1H), 7.19 (d, J = 6.8 Hz, 1H), 7.26 (t, J = 7.2 Hz, 1H), 7.32 (d, J = 8.0 Hz, 2H), 7.35- 7.47 (m, 4H), 7.56-7.60 (t, J = 7.6 Hz, 1H), 7.78-7.83 (m, 2H), 7.95 (s, 1H); 1H merged with DMSO residual peak. HPLC: 98.19%. |
| 8 | 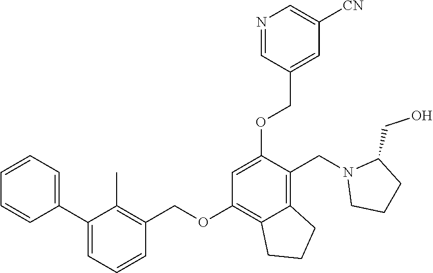 [SMILES: Cc1c(COc2cc(OCc3cncc(C#N)c3)c(CN3CCC[C@H]3CO)c3c2CCC3)cccc1-c1ccccc1] | LCMS (ES) m/z = 560.4 [M + H]⁺; ¹H NMR (400 MHz, DMSO-d₆) δ ppm: 1.48-1.60 (m, 3H), 1.75-1.85 (m, 1H), 1.90-2.02 (m, 2H), 2.12- 2.22 (s, 4H), 2.60-2.78 (m, 3H), 2.80-3.00 (m, 2H), 3.10-3.20 (m, 1H), 3.36-30.46 (m, 2H), 3.78-3.85 (m, 1H), 4.20 (bs, 1H), 5.13 (m, 2H), 5.20-5.34 (m, 2H), 6.74 (s, 1H), 7.19 (d, J = 7.4 Hz, 1H), 7.24- 7.40 (m, 4H), 7.42-7.48 (m, 3H), 8.43 (s, 1H), 8.90 (s, 2H); 1H merged with DMSO residual peak. HPLC: 98.19%. |
| 9 | 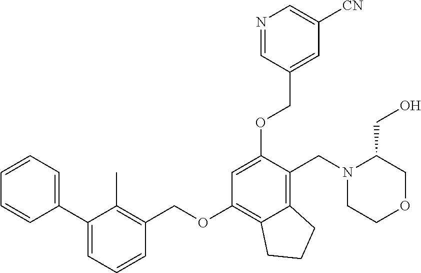 [SMILES: Cc1c(COc2cc(OCc3cncc(C#N)c3)c(CN3CCOC[C@H]3CO)c3c2CCC3)cccc1-c1ccccc1] | LCMS (ES) m/z = 576.46 [M + H]⁺; ¹H NMR (400 MHz, DMSO-d₆) δ ppm: 9.02 (d, J = 2.0 Hz, 1H), 8.99 (d, J = 2 Hz, 1H), 8.42 (s, 1H), 7.47- 7.44 (m, 3H), 7.39-7.36 (m, 1H), 7.32 (d, J = 6.8 Hz, 2H), 7.26 (t, J = 7.6 Hz, 1H), 7.19 (d, J = 6.4 Hz, 1H), 6.75 (s, 1H), 5.31-5.23 (m, 2H), 5.13 (s, 2H), 4.47 (bs, 1H), 3.92 (d, J = 12 Hz, 1H), 3.73-3.70 (m, 2H), 3.54-3.52 (m, 1H), 3.27- 3.18 (m, 3H), 2.97-2.80 (m, 2H), 2.74 (t, J = 7.6 Hz, 2H), 2.20 (s, 3H), 2.08-2.03 (m, 1H), 2.00-1.93 (m, 2H). HPLC: 96.57%. |
| 10 | 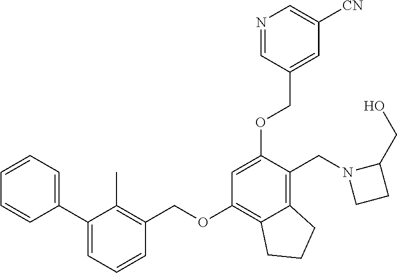 [SMILES: Cc1c(COc2cc(OCc3cncc(C#N)c3)c(CN3CCC3CO)c3c2CCC3)cccc1-c1ccccc1] | LCMS (ES) m/z = 546.41 [M + H]⁺; ¹H NMR (400 MHz, DMSO-d₆) δ ppm: 9.00 (s, 2H), 8.51 (s, 1H), 7.46 (t, J = 8 Hz, 3H), 7.38 (t, J = 7.2 Hz, 1H), 7.32 (d, J = 7.2 Hz, 2H), 7.27 (t, J = 7.6 Hz, 1H), 7.19 (d, J = 7.2 Hz, 1H), 6.74 (s, 1H), 5.30-5.21 (m, 2H), 5.14 (s, 2H), 4.24 (bs, 1H), 3.58 (d, J = 11.6 Hz, 1H), 3.50-3.45 (m, 1H), 3.21 (bs, 3H), 3.05-3.03 (m, 1H), 2.98-2.78 (m, 3H), 2.74 (t, J = 7.6 Hz, 2H), 2.20 (s, 3H), 2.00- 1.90 (m, 2H), 1.89-1.80 (m, 1H), 1.75-1.71 (m, 1H). HPLC: 95.55%. |
| 11 | 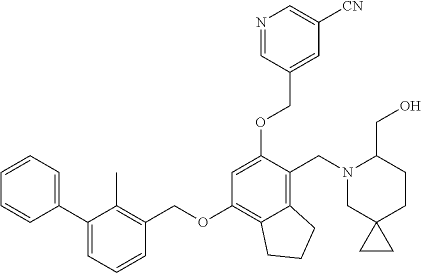 [SMILES: Cc1c(COc2cc(OCc3cncc(C#N)c3)c(CN3CC4(CCC3CO)CC4)c3c2CCC3)cccc1-c1ccccc1] | LCMS (ES) m/z = 600.45 [M + H]⁺; ¹H NMR (400 MHz, DMSO-d₆) δ ppm: 9.00 (d, J = 2.0 Hz, 2H), 8.42 (s, 1H), 7.46 (t, J = 7.6 Hz, 3H), 7.39-7.36 (m, 1H), 7.33-7.31 (m, 2H), 7.27 (t, J = 7.6 Hz, 1H), 7.19 (d, J = 6.4 Hz, 1H), 6.72 (s, 1H), 5.25 (s, 2H), 5.13 (s, 2H), 4.28 (t, J = 5.2 Hz, 1H), 4.00 (d, J = 12.4 Hz, 1H), 3.68-3.64 (m, 1H), 3.46- 3.40 (m, 1H), 3.23 (d, J = 12.4 Hz, 1H), 3.0-2.91 (m, 1H), 2.89-2.79 (m, 1H), 2.73 (t, J = 7.6 Hz, 2H), 2.28-2.22 (m, 1H), 2.21 (s, 3H), 2.10-2.01 (m, 2H), 1.98-1.92 (m, 2H), 1.79-1.70 (m, 1H), 1.49-1.43 (m, 1H), 1.42-1.37 (m, 1H), 1.25- |
| | | 1.15 (m, 1H), 0.18-0.12 (m, 3H), |
| | | −0.00-−0.03 (m, 1H). HPLC: |
| | | 97.83%. |
| 12 | 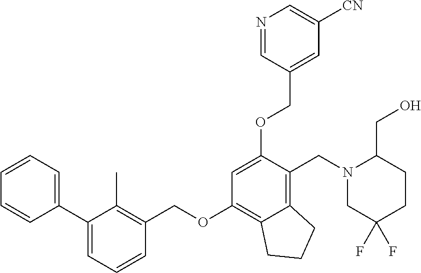 [SMILES: Cc1c(COc2cc(OCc3cncc(C#N)c3)c(CN3CC(F)(F)CCC3CO)c3c2CCC3)cccc1-c1ccccc1] | LCMS (ES) m/z = 610.30 [M + H]⁺; ¹H NMR (400 MHz, DMSO-d₆) δ ppm: 8.99 (dd, J = 8.4, 2.0 Hz, 2H), 8.39 (s, 1H), 7.48-7.44 (m, 3H), 7.38 (t, J = 7.2 Hz, 1H), 7.33-7.31 (m, 2H), 7.28 (t, J = 7.2 Hz, 1H), 7.20 (d, J = 7.2 Hz, 1H), 6.77 (s, 1H), 5.26 (s, 2H), 5.16 (s, 2H), 4.54 (t, J = 5.2 Hz, 1H), 3.94 (d, J = 12.0 Hz, 1H), 3.69-3.64 (m, 1H), 3.49- 3.43 (m, 1H), 3.40 (d, J = 12.4 Hz, 1H), 2.92-2.81 (m, 2H), 2.73 (t, J = 7.2 Hz, 3H), 2.44-2.38 (m, 2H), 2.21 (s, 3H), 2.02-1.92 (m, 3H), 1.85-1.73 (m, 2H), 1.62-1.53 (m, 1H). HPLC: 99.76%. |
| 13 | 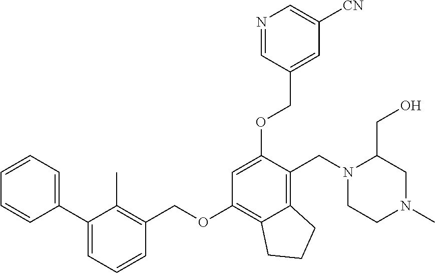 [SMILES: Cc1c(COc2cc(OCc3cncc(C#N)c3)c(CN3CCN(C)CC3CO)c3c2CCC3)cccc1-c1ccccc1] | LCMS (ES) m/z = 589.45 [M + H]⁺; ¹H NMR (400 MHz, DMSO-d₆) δ ppm: 9.00 (d, J = 7.2 Hz, 2H), 8.41 (s, 1H), 7.47-7.44 (m, 3H), 7.39- 7.37 (m, 1H), 7.32 (d, J = 7.2 Hz, 2H), 7.26 (t, J = 7.4 Hz, 1H), 7.19 (d, J = 7.6 Hz, 1H), 6.74 (s, 1H), 5.31-5.26 (m, 2H), 5.13 (s, 2H), 4.41 (bs, 1H), 3.92 (d, J = 12.4 Hz, 1H), 3.73 (d, J = 9.6 Hz, 1H), 3.41- 3.38 (m, 2H), 3.18 (d, J = 12.4 Hz, 1H), 2.98-2.82 (m, 2H), 2.73 (t, J = 7.2 Hz, 2H), 2.62-2.58 (m, 2H), 2.33 (bs, 1H), 2.20 (s, 3H), 2.09 (bs, 4H), 2.00-1.93 (m, 4H). HPLC: 98.29%. |
| 14 | 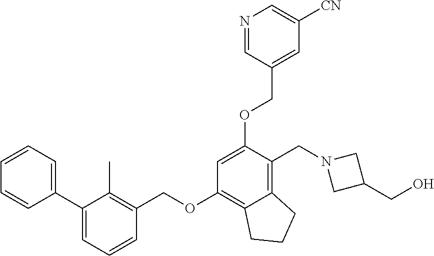 [SMILES: Cc1c(COc2cc(OCc3cncc(C#N)c3)c(CN3CC(CO)C3)c3c2CCC3)cccc1-c1ccccc1] | LCMS (ES) m/z = 546.45 [M + H]⁺; ¹H NMR (400 MHz, DMSO-d₆) δ ppm: 9.00 (s, 2H), 8.49 (s, 1H), 7.47-7.43 (m, 3H), 7.39-7.36 (m, 1H), 7.32 (d, J = 6.8 Hz, 2H), 7.26 (t, J = 7.6 Hz, 1H), 7.19 (d, J = 7.6 Hz, 1H), 6.75 (s, 1H), 5.28 (s, 2H), 5.14 (s, 2H), 4.56 (s, 1H), 3.56 (bs, 2H), 3.43 (d, J = 6.4 Hz, 2H), 3.25 (bs, 2H), 2.95 (bs, 2H), 2.89 (t, J = 7.2 Hz, 2H), 2.74 (t, J = 7.6 Hz, 2H), 2.46-2.40 (m, 1H), 2.20 (s, 3H), 2.01-1.95 (m, 2H). HPLC: 98.21%. |
| 15 | 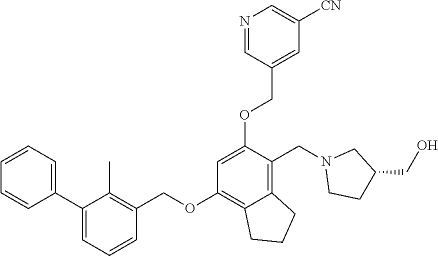 [SMILES: Cc1c(COc2cc(OCc3cncc(C#N)c3)c(CN3CC[C@@H](CO)C3)c3c2CCC3)cccc1-c1ccccc1] | LCMS (ES) m/z = 560.36 [M + H]⁺; ¹H NMR (400 MHz, DMSO-d₆) δ ppm: 8.99 (s, 2H), 8.41 (s, 1H), 8.20 (s, 1H), 7.47-7.44 (m, 2H), 7.39- 7.36 (m, 1H), 7.32 (d, J = 7.2 Hz, 2H), 7.27 (t, J = 7.6 Hz, 1H), 7.19 (d, J = 7.6 Hz, 1H), 6.76 (s, 1H), 5.27 (s, 2H), 5.14 (s, 2H), 3.52 (s, 2H), 3.25-3.19 (m, 3H), 2.89 (t, J = 7.2 Hz, 2H), 2.75 (t, J = 7.6 Hz, 2H), 2.44 (bs, 2H), 2.29-2.26 (m, 1H), 2.21 (s, 3H), 2.16-2.13 (m, 1H), 2.01-2.95 (m, 2H), 1.79-1.75 (m, 1H), 1.36-1.31 (m, 1H), 1.17- 1.13 (m, 1H). HPLC: 96.1%. |
| 16 | 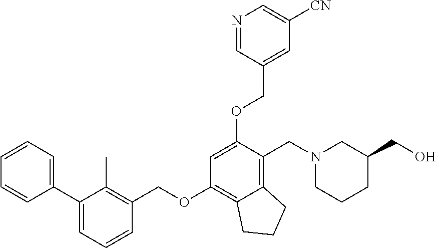 [SMILES: Cc1c(COc2cc(OCc3cncc(C#N)c3)c(CN3CCC[C@H](CO)C3)c3c2CCC3)cccc1-c1ccccc1] | LCMS (ES) m/z = 574.40 [M + H]⁺; ¹H NMR (400 MHz, DMSO-d₆) δ ppm: 9.02 (s, 1H), 8.99 (s, 1H), 8.39 (s, 1H), 7.47-7.44 (m, 3H), 7.37 (t, J = 7.2 Hz, 1H), 7.32 (d, J = 7.2 Hz, 2H), 7.27 (t, J = 7.6 Hz, 1H), 7.19 (d, J = 7.2 Hz, 1H), 6.76 (s, 1H), 5.27 (s, 2H), 5.14 (s, 2H), 4.35 (t, J = 5.6 Hz, 1H), 3.29-3.14 (m, 4H), 2.87 (t, J = 7.2 Hz, 2H), 2.83-2.81 (m, 1H), 2.75 (t, J = 7.2 Hz, 2H), 2.64 (bs, 1H), 2.21 (s, 3H), 2.05- 1.85 (m, 3H), 1.65-1.48 (m, 4H), 1.45-1.33 (m, 1H), 0.90-0.82 (m, 1H). HPLC: 96.05%. |
| 17 | 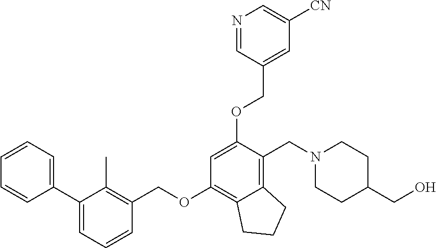 [SMILES: Cc1c(COc2cc(OCc3cncc(C#N)c3)c(CN3CCC(CO)CC3)c3c2CCC3)cccc1-c1ccccc1] | LCMS (ES) m/z = 574.35 [M + H]⁺; ¹H NMR (400 MHz, DMSO-d₆) δ ppm: 9.01 (dd, J = 14.0, 1.6 Hz, 2H), 8.39 (t, J = 2.8 Hz, 1H), 7.48- 7.44 (m, 3H), 7.41-7.36 (m, 1H), 7.33-7.31 (m, 2H), 7.27 (t, J = 7.6 Hz, 1H), 7.19 (d, J = 6.8 Hz, 1H), 6.76 (s, 1H), 5.28 (s, 2H), 5.14 (s, 2H), 4.36 (t, J = 5.2 Hz, 1H), 3.35 (bs, 2H), 3.20 (t, J = 6 Hz, 2H), 2.87 (t, J = 7.2 Hz, 2H), 2.76-2.73 (m, 3H), 2.21 (s, 3H), 2.00-1.86 (m, 5H), 1.59 (d, J = 11.2 Hz, 2H), 1.30 (bs, 1H), 1.05-0.99 (m, 2H). HPLC: 95.39%. |
| 18 | 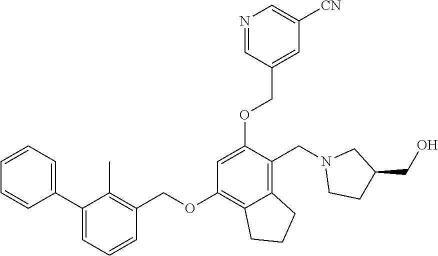 [SMILES: Cc1c(COc2cc(OCc3cncc(C#N)c3)c(CN3CC[C@H](CO)C3)c3c2CCC3)cccc1-c1ccccc1] | LCMS (ES) m/z = 560.30 [M + H]⁺; ¹H NMR (400 MHz, DMSO-d₆) δ ppm: 8.99 (t, J = 2.4 Hz, 2H), 8.41 (t, J = 1.6 Hz, 1H), 7.47-7.44 (m, 3H), 7.39-7.36 (m, 1H), 7.33-7.31 (m, 2H), 7.27 (t, J = 7.6 Hz, 1H), 7.20-7.18 (m, 1H), 6.75 (s, 1H), 5.27 (t, J = 13.6 Hz, 2H), 5.14 (s, 2H), 4.47 (bs, 1H), 3.49 (bs, 2H), 3.27-3.18 (m, 2H), 2.88 (t, J = 7.2 Hz, 2H), 2.75 (t, J = 7.2 Hz, 2H), 2.60 (bs, 1H), 2.45-2.32 (m, 2H), 2.28-2.20 (m, 4H), 2.15 (bs, 1H), 2.02-1.93 (m, 2H), 1.80-1.72 (m, 1H), 1.36-1.30 (m, 1H). HPLC: 96.34%. |
| 19 | 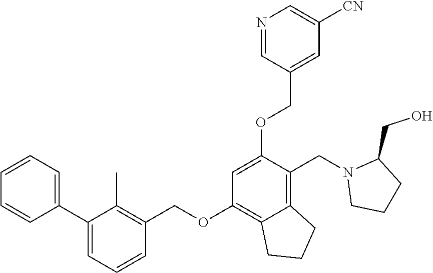 [SMILES: Cc1c(COc2cc(OCc3cncc(C#N)c3)c(CN3CCC[C@@H]3CO)c3c2CCC3)cccc1-c1ccccc1] | LCMS (ES) m/z = 560.35 [M + H]⁺; ¹H NMR (400 MHz, DMSO-d₆) δ ppm: 8.98 (d, J = 1.6 Hz, 2H), 8.43 (t, J = 2.0 Hz, 1H), 7.47-7.44 (m, 3H), 7.39-7.37 (m, 1H), 7.33-7.31 (m, 2H), 7.26 (t, J = 7.6 Hz, 1H), 7.19 (d, J = 6.8 Hz, 1H), 6.74 (s, 1H), 5.31-5.22 (m, 2H), 5.13 (t, J = 13.2 Hz, 2H), 4.21 (t, J = 5.2 Hz, 1H), 3.82 (d, J = 12 Hz, 1H), 3.43- 3.34 (m, 2H), 3.18-3.13 (m, 2H), 2.98-2.83 (m, 2H), 2.74 (t, J = 7.2 Hz, 2H), 2.66-2.64 (m, 1H), 2.20- 2.14 (m, 4H), 2.00-1.92 (m, 2H), 1.83-1.79 (m, 1H), 1.57-1.47 (m, 3H). HPLC: 95.73%. |
| 20 | 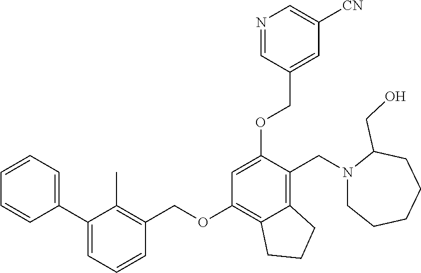 [SMILES: Cc1c(COc2cc(OCc3cncc(C#N)c3)c(CN3CCCCCC3CO)c3c2CCC3)cccc1-c1ccccc1] | LCMS (ES) m/z = 588.39 [M + H]⁺; ¹H NMR (400 MHz, DMSO-d₆) δ ppm: 8.99 (d, J = 10 Hz, 2H), 8.42 (s, 1H), 7.51-7.46 (m, 3H), 7.40- 7.22 (m, 4H), 7.19 (d, J = 7.6 Hz, 1H), 6.74 (s, 1H), 5.30-5.22 (m, 2H), 5.14 (s, 2H), 4.05 (bs, 1H), 3.75 (d, J = 11.6 Hz, 1H), 3.59 (d, J = 11.6 Hz, 1H), 3.10-3.06 (m, 1H), 2.89 (t, J = 6.4 Hz, 2H), 2.74 (t, J = 6.4 Hz, 2H), 2.58 (bs, 2H), 2.21 (s, 3H), 1.98 (t, J = 1.98 Hz, 2H), 1.95 (s, 1H), 1.83 (bs, 1H), 1.66 (bs, 1H), 1.55 (bs, 2H), 1.45- 1.20 (m, 5). HPLC: 90.29%. |
| 21 | 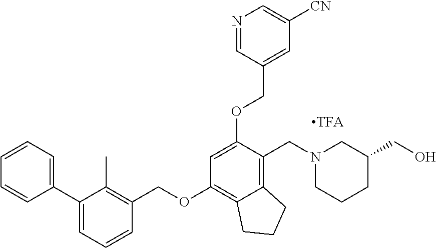 [SMILES: Cc1c(COc2cc(OCc3cncc(C#N)c3)c(CN3CCC[C@@H](CO)C3)c3c2CCC3)cccc1-c1ccccc1] | LCMS (ES) m/z = 574.48 [M + H]⁺; ¹H NMR (400 MHz, DMSO-d₆) δ ppm: 9.04 (t, J = 2.0 Hz, 2H), 8.59 (bs, 1H), 8.49 (t, J = 2.0 Hz, 1H), 7.48-7.45 (m, 3H), 7.38 (t, J = 7.2 Hz, 1H), 7.34-7.19 (m, 4H), 6.91 (s, 1H), 5.34 (s, 2H), 5.24 (s, 2H), 4.77 (bs, 1H), 4.19 (d, J = 4.4 Hz, 2H), 3.45-3.34 (m, 3H), 3.22-3.19 (m, 1H), 3.01-2.84 (m, 3H), 2.83-2.71 (m, 3H), 2.22 (s, 3H), 2.07-2.00 (m, 2H), 1.92-1.76 (m, 2H), 1.64 (bs, 2H), 1.14-1.04 (m, 1H). HPLC: 97.05%. |
| 22 | 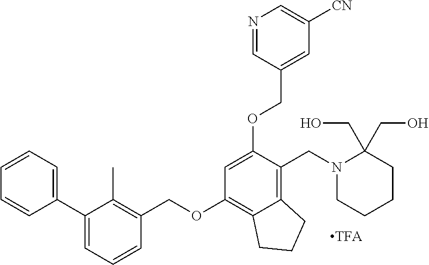 [SMILES: Cc1c(COc2cc(OCc3cncc(C#N)c3)c(CN3CCCCC3(CO)CO)c3c2CCC3)cccc1-c1ccccc1] | LCMS (ES) m/z = 604.52 [M + H]⁺; ¹H NMR (400 MHz, DMSO-d₆) δ ppm: 9.07-8.98 (m, 2H), 8.49 (t, J = 8 Hz, 1H), 7.88 (bs, 1H), 7.50-7.41 (m, 3H), 7.36 (d, J = 5.6 Hz, 1H), 7.32 (d, J = 7.2 Hz, 2H), 7.28 (dd, J = 8.8 Hz, 1H), 7.21 (t, J = 7.6 Hz, 1H), 6.87 (s, 1H), 5.58 (bs, 1H), 5.40-5.30 (m, 3H), 5.22 (s, 2H), 4.56 (d, J = 12.8 Hz, 1H), 4.15-4.05 (m, 1H), 3.92-3.80 (m, 3H), 3.60- 3.40 (m, 1H), 3.20-3.10 (m, 2H), 3.09-3.00 (m, 1H), 2.90-2.82 (m, 1H), 2.75 (t, J = 7.6 Hz, 2H), 2.21 (s, 3H), 2.08-2.00 (m, 2H), 1.85- 1.76 (m, 1H), 1.72-1.60 (m, 4H), 1.55-1.50 (m, 1H). HPLC: 94.02%. |

### Example 23: Synthesis of (S)-3-(((7-((2-(hydroxymethyl)piperidin-1-yl)methyl)-6-(2,2,2-trifluoroethoxy)-2,3-dihydro-1H-inden-4-yl)oxy)methyl)-[1,1′-biphenyl]-2-carbonitrile

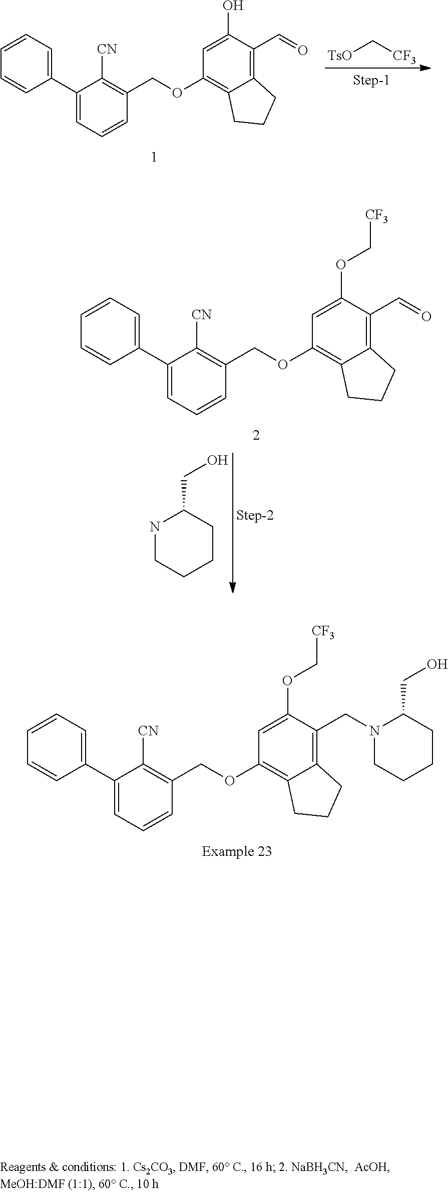

### Step-1: Synthesis of 3-(((7-formyl-6-(2,2,2-trifluoroethoxy)-2,3-dihydro-1H-inden-4-yl)oxy)methyl)-[1,1′-biphenyl]-2-carbonitrile (2)

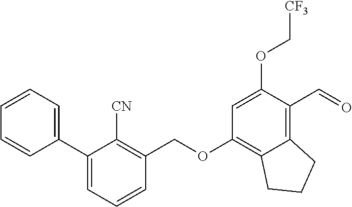 [SMILES: N#Cc1c(COc2cc(OCC(F)(F)F)c(C=O)c3c2CCC3)cccc1-c1ccccc1]

To a solution of 3-(((7-formyl-6-hydroxy-2,3-dihydro-1H-inden-4-yl)oxy)methyl)-[1,1′-biphenyl]-2-carbonitrile (1, 0.50 g, 1.35 mmol) and 2,2,2-trifluoroethyl 4-methylbenzenesulfonate (1.0 g, 4 mmol) in N,N-dimethylformamide (8 mL), cesium carbonate (0.39 g, 2.0 mmol) was added and the mixture was heated at 60° C. for 16 h. After completion of the reaction, the reaction mixture was diluted with water (20 mL) and extracted with ethyl acetate (3×25 mL). The combined organic layer was dried over anhydrous sodium sulfate and concentrated. The crude was purified by silica gel flash column chromatography using 20% ethyl acetate in hexanes as eluent to obtain the desired product (2, 0.3 g, 47%) as a white solid. LCMS (ES) m/z=452.57 [M+H]⁺;

### Step-2: Synthesis of (S)-3-(((7-((2-(hydroxymethyl)piperidin-1-yl)methyl)-6-(2,2,2-trifluoroethoxy)-2,3-dihydro-1H-inden-4-yl)oxy)methyl)-[1,1′-biphenyl]-2-carbonitrile (Example 23)

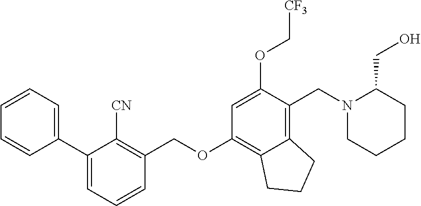 [SMILES: N#Cc1c(COc2cc(OCC(F)(F)F)c(CN3CCCC[C@H]3CO)c3c2CCC3)cccc1-c1ccccc1]

A solution of 3-(((7-formyl-6-(2,2,2-trifluoroethoxy)-2,3-dihydro-1H-inden-4-yl)oxy)methyl)-[1,1′-biphenyl]-2-carbonitrile (2, 0.15 g, 0.34 mmol), (S)-piperidin-2-ylmethanol (0.114 g, 0.94 mmol), sodium cyanoborohydride (0.061 g, 0.9 mmol) and acetic acid (2 drops) in methanol (3 mL) and N, N-dimethylformamide (3 mL) was heated at 60° C. for 10 h. After completion of the reaction, the reaction mixture was diluted with water (10 mL) and extracted with 10% methanol in dichloromethane (3×25 mL). The combined organic layer was dried over anhydrous sodium sulfate and concentrated. The resulting crude was purified by silica gel flash column chromatography using 10% methanol in dichloromethane as eluent to obtain desired compound. The compound was again purified by reverse phase prep-HPLC (ammonium acetate buffer) to afford the title product (Example 23, 0.020 g, 11%) as white solid.

LCMS (ES) m/z=551.37 [M+H]⁺; ¹H NMR (400 MHz, DMSO-d₆) δ ppm: 1.18-1.30 (m, 4H), 1.32-1.42 (m, 1H), 1.55-1.62 (m, 1H), 1.65-1.70 (m, 1H), 1.90-2.02 (m, 3H), 2.22 (bs, 1H), 2.76 (t, J=7.6 Hz, 2H), 2.80-2.90 (m, 1H), 2.92-3.02 (m, 1H), 3.22 (d, J=12.4 Hz, 1H), 3.37-3.43 (m, 1H), 3.70-3.76 (m, 1H), 3.92 (d, J=12.0 Hz, 1H), 4.36 (t, J=4.8 Hz, 1H), 4.67-4.76 (m, 2H), 5.32 (s, 2H), 6.75 (s, 1H), 7.50-7.64 (m, 6H), 7.76 (d, J=7.6 Hz, 1H), 7.84 (t, J=7.6 Hz, 1H); HPLC purity 97.61%.

### Example 24: Synthesis of (S)-(1-((7-((2-methyl-[1,1′-biphenyl]-3-yl)methoxy)-5-(2,2,2-trifluoroethoxy)-2,3-dihydro-1H-inden-4-yl)methyl)piperidin-2-yl)methanol

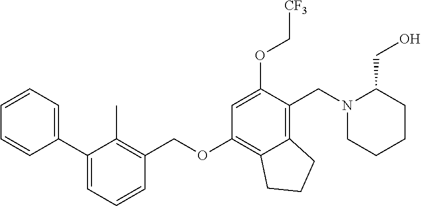 [SMILES: Cc1c(COc2cc(OCC(F)(F)F)c(CN3CCCC[C@H]3CO)c3c2CCC3)cccc1-c1ccccc1]

The Example 24 was prepared by procedure similar to the one described in Example 23 by using 5-hydroxy-7-((2-methyl-[1,1′-biphenyl]-3-yl)methoxy)-2,3-dihydro-1H-indene-4-carbaldehyde as starting material. LCMS (ES) m/z=540.39 [M+H]⁺; ¹H NMR (400 MHz, DMSO-d₆) δ ppm: 1.20-1.34 (m, 2H), 1.55-1.70 (m, 1H), 1.85 (s, 2H), 1.95-2.05 (m, 2H), 2.12 (s, 3H), 2.21 (s, 3H), 2.72-2.76 (m, 2H), 2.85-2.90 (m, 2H), 2.96-3.00 (m, 1H), 3.30 (s, 1H), 3.42-3.48 (m, 1H), 3.70-3.75 (m 1H), 3.87-3.92 (m, 1H), 4.35 (bs, 1H), 4.68-4.76 (m, 2H), 5.14 (s, 2H), 6.77 (d, J=9.0 Hz, 1H), 7.21 (d, J=6.4 Hz, 1H), 7.28-7.36 (m, 3H), 7.37-7.42 (m, 1H), 7.44-7.51 (m, 3H); HPLC: 99.57%.

### Example 25: Synthesis of (S)-5-(((4-((6-(hydroxymethyl)-5-azaspiro[2.4]heptan-5-yl)methyl)-7-((2-methyl-[1,1′-biphenyl]-3-yl)methoxy)-2,3-dihydro-1H-inden-5-yl)oxy)methyl)nicotinonitrile

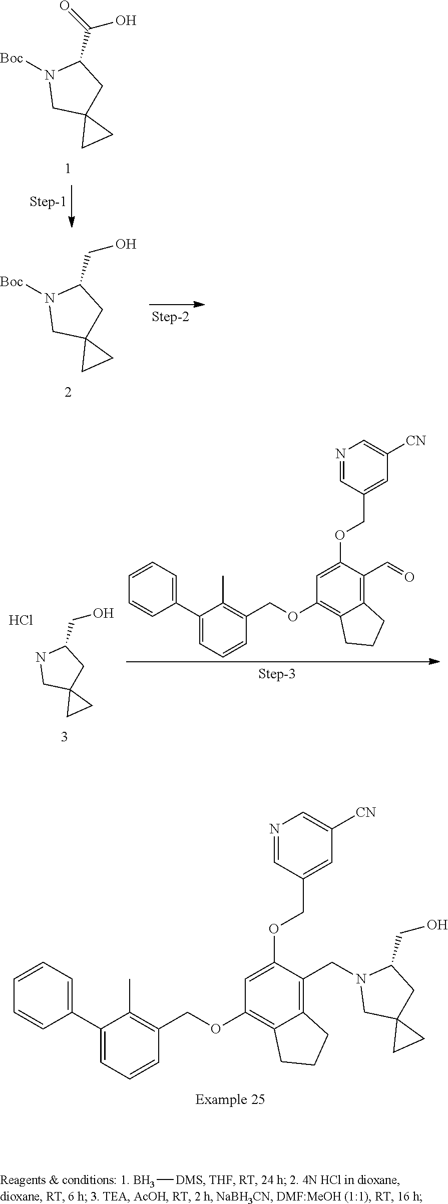

### Step-1: Synthesis of tert-butyl (S)-6-(hydroxymethyl)-5-azaspiro[2.4]heptane-5-carboxylate (2)

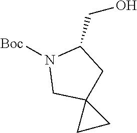 [SMILES: CC(C)(C)OC(=O)N1CC2(CC2)C[C@H]1CO]

A solution of (S)-5-(tert-butoxycarbonyl)-5-azaspiro[2.4]heptane-6-carboxylic acid (1, 0.8 g, 3.3 mmol) in dry tetrahydrofuran (15 mL) at 0° C. was treated with borane-DMS (6.6 mL, 1M in tetrahydrofuran, 2 eq) and the reaction mixture was stirred at room temperature for 24 h. After completion of the reaction, the reaction mixture was quenched with methanol (20 mL) and concentrated under vacuum. The residue was diluted with dichloromethane (100 mL) and was washed with water (80 mL), saturated sodium bicarbonate solution (80 mL), brine (80 mL) and concentrated under reduced pressure to obtain the desired product (2, 0.73 g, 96%) as light yellow liquid.

### Step-2: Synthesis of (S)-(5-azaspiro[2.4]heptan-6-yl)methanol hydrochloride (3)

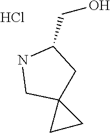

To a solution of tert-butyl (S)-6-(hydroxymethyl)-5-azaspiro[2.4]heptane-5-carboxylate (2, 0.73 g, 3.2 mmol) in 1,4-dioxane (25 mL), 4N hydrochloric acid in 1,4-dioxane (2.5 mL) was added. The reaction mixture was stirred at room temperature for 6 h. After completion of the reaction, the reaction mixture was concentrated to obtain the desired product (3, 0.53 g, 98.3%) as light yellow solid.

### Step-3: Synthesis of (S)-5-(((4-((6-(hydroxymethyl)-5-azaspiro[2.4]heptan-5-yl)methyl)-7-((2-methyl-[1,1′-biphenyl]-3-yl)methoxy)-2,3-dihydro-1H-inden-5-yl)oxy)methyl)nicotinonitrile (Example 25)

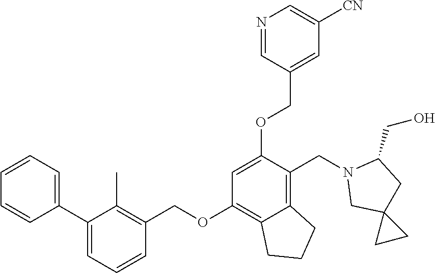 [SMILES: Cc1c(COc2cc(OCc3cncc(C#N)c3)c(CN3CC4(CC4)C[C@H]3CO)c3c2CCC3)cccc1-c1ccccc1]

To a solution of 5-(((4-formyl-7-((2-methyl-[1,1′-biphenyl]-3-yl)methoxy)-2,3-dihydro-1H-inden-5-yl)oxy)methyl)nicotinonitrile (0.3 g, 0.632 mmol) in N,N-dimethylformamide (5 mL) and methanol (5 mL), (S)-(5-azaspiro[2.4]heptan-6-yl)methanol hydrogen chloride (3, 155 mg, 0.94 mmol), triethylamine (0.096 g, 0.94 mmol) and acetic acid (2 drops) were added and stirred for 2 h. To this mixture, sodium cyanoborohydride (0.119 g, 1.8 mmol) was added and the reaction mixture was stirred at room temperature for 16 h. After completion of the reaction, the reaction mixture was diluted with water (15 mL) and extracted with 10% methanol in dichloromethane (3×30 mL). The combined organic layer was dried over anhydrous sodium sulphate and concentrated. The resulting crude was purified by silica gel flash column chromatography using 0-10% methanol in dichloromethane as eluent to afford the title product (Example 25, 0.05 g, 13.5%) as white solid. LCMS (ES) m/z=586.74 [M+H]⁺; ¹H NMR (400 MHz, DMSO-d₆) δ ppm: 0.30-0.45 (m, 4H), 1.45-1.55 (m, 1H), 1.86-2.00 (m, 3H), 2.20 (s, 3H), 2.45 (m, 1H), 2.60-3.00 (m, 5H), 3.25-3.39 (m, 2H), 3.50-3.55 (m, 1H), 3.80-3.86 (m, 1H), 4.25 (bs, 1H), 5.13 (s, 2H), 5.20-5.30 (m, 2H), 6.73 (s, 1H), 7.18 (d, J=7.6 Hz, 1H), 7.26-7.40 (m, 4H), 7.42-7.48 (m, 3H), 8.41 (s, 1H), 8.89-9.02 (m, 2H). 1H merged with DMSO residual peak. HPLC: 99.47%.

### Example 26: Synthesis (S)-5-(((7-((2-cyano-[1,1′-biphenyl]-3-yl)methoxy)-4-((6-(hydroxymethyl)-5-azaspiro[2.4]heptan-5-yl)methyl)-2,3-dihydro-1H-inden-5-yl)oxy)methyl)nicotinonitrile

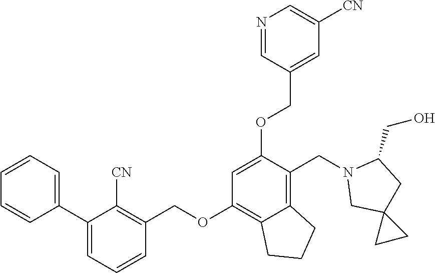 [SMILES: N#Cc1cncc(COc2cc(OCc3cccc(-c4ccccc4)c3C#N)c3c(c2CN2CC4(CC4)C[C@H]2CO)CCC3)c1]

The Example-26 was prepared by a procedure similar to the one described in Example-25 by using 5-(((7-((2-cyano-[1,1′-biphenyl]-3-yl)methoxy)-4-formyl-2,3-dihydro-1H-inden-5-yl)oxy)methyl)nicotinonitrile as starting material. LCMS (ES) m/z=597.36 [M+H]⁺; ¹H NMR (400 MHz, DMSO-d₆) δ ppm: 0.32-0.47 (m, 4H), 1.45-1.53 (m, 1H), 1.84-2.02 (m, 3H), 2.30-2.35 (m, 1H), 2.43-2.48 (m, 2H), 2.70-2.80 (m, 2H), 2.80-3.01 (m, 2H), 3.23-3.30 (m, 1H), 3.35-3.40 (m, 1H), 3.47-3.55 (m, 1H), 3.83 (d, J=12.0 Hz, 1H), 4.24 (bs, 1H), 5.20-5.5.29 (m, 2H), 5.31 (s, 2H), 6.73 (s, 1H), 7.50-7.62 (m, 6H), 7.70 (d, J=7.6 Hz, 1H), 7.79 (t, J=8.0 Hz, 1H), 8.40 (s, 1H) 8.98 (dd, J=8.0, 1.6 Hz, 2H). HPLC: 98.51%.

### Example 27: Synthesis of (S)-(1-((5-(2-fluoroethoxy)-7-((2-methyl-[1,1′-biphenyl]-3-yl)methoxy)-2,3-dihydro-1H-inden-4-yl)methyl)piperidin-2-yl)methanol

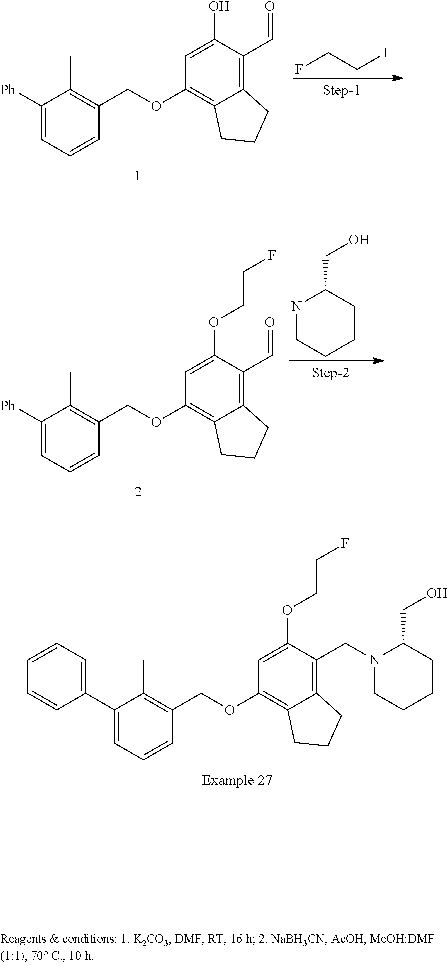

### Step-1: Synthesis of 5-(2-fluoroethoxy)-7-((2-methyl-[1,1′-biphenyl]-3-yl)methoxy)-2,3-dihydro-1H-indene-4-carbaldehyde (2)

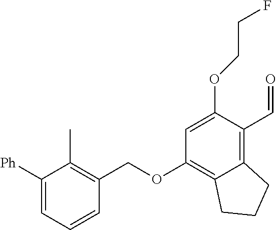 [SMILES: Cc1c(COc2cc(OCCF)c(C=O)c3c2CCC3)cccc1-c1ccccc1]

To a solution of 5-hydroxy-7-((2-methyl-[1,1′-biphenyl]-3-yl)methoxy)-2,3-dihydro-1H-indene-4-carbaldehyde (1, 0.7 g, 1.95 mmol) in N,N-dimethylformamide (20 mL), potassium carbonate (0.958 g, 6.84 mmol) and 1-fluoro-2-iodoethane (0.51 g, 2.93 mmol) were added. The reaction mixture was stirred at room temperature for 16 h. After completion of the reaction, the reaction mixture was diluted with water (20 mL) and extracted with ethyl acetate (3×20 mL). The combined organic layer was dried over sodium sulfate and concentrated. The resulting crude was purified by silica gel flash column chromatography using 0-50% ethyl acetate in hexane as eluent to afford the desired product (2, 0.47 g, 58.5%) as white solid. LCMS (ES) m/z=405.08 [M+H]⁺.

### Step-2: Synthesis of (S)-(1-((5-(2-fluoroethoxy)-7-((2-methyl-[1,1′-biphenyl]-3-yl)methoxy)-2,3-dihydro-1H-inden-4-yl)methyl)piperidin-2-yl)methanol (Example 27)

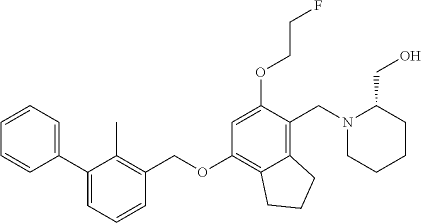 [SMILES: Cc1c(COc2cc(OCCF)c(CN3CCCC[C@H]3CO)c3c2CCC3)cccc1-c1ccccc1]

A solution of 5-(2-fluoroethoxy)-7-((2-methyl-[1,1′-biphenyl]-3-yl)methoxy)-2,3-dihydro-1H-indene-4-carbaldehyde (2, 0.225 g, 0.55 mmol), (S)-piperidin-2-ylmethanol (0.096 g, 0.83 mmol), sodium cyanoborohydride (0.107 g, 1.67 mmol) and acetic acid (2 drops) in methanol (4 mL) and N,N-dimethylformamide (4 mL) was heated at 70° C. for 10 h. After completion of the reaction, the reaction mixture was diluted with water (10 mL) and extracted with 10% methanol in dichloromethane (3×35 mL). The combined organic layer was dried over anhydrous sodium sulfate and concentrated. The resulting crude was purified by silica gel flash column chromatography using 10% methanol in dichloromethane as eluent to afford the title product (Example 27, 0.040 g, 14.28%) as white solid. LCMS (ES) m/z=504.23 [M+H]⁺; ¹H NMR (400 MHz, DMSO-d₆) δ ppm: 1.15-1.30 (m, 3H), 1.35-1.43 (m, 1H), 1.55-1.70 (m, 2H), 1.90-2.02 (m, 3H), 2.21 (s, 4H), 2.57-2.61 (m, 1H), 2.70-2.78 (m, 2H), 2.80-2.90 (m, 1H), 2.90-3.00 (m, 1H), 3.18-3.24 (m, 1H), 3.40-3.48 (m, 1H), 3.66-3.76 (m, 1H), 3.85-3.93 (m, 1H), 4.16-4.36 (m, 3H), 4.65-4.82 (m, 2H), 5.14 (s, 2H), 6.64 (s, 1H), 7.20 (d, J=7.4 Hz 1H), 7.26-7.34 (m, 3H), 7.36-7.40 (m, 1H), 7.44-7.50 (m, 3H); HPLC: 97.24%.

The compounds listed in below Table-2 were prepared by a procedure similar to the one described in Example-27 with appropriate variations in reactants, quantity of reagents, protections & deprotections, solvents & reaction conditions. The characterization data of the compounds are summarized herein below table.

TABLE 2

| Example | | |
|---|---|---|
| No. | Structure | Characterization data |
| 28 | 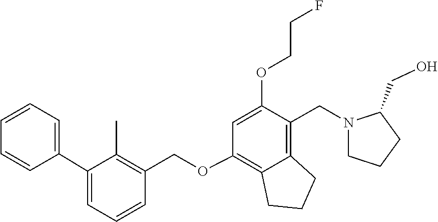 [SMILES: Cc1c(COc2cc(OCCF)c(CN3CCC[C@H]3CO)c3c2CCC3)cccc1-c1ccccc1] | LCMS (ES) m/z = 490.17 [M + H]⁺; ¹H NMR (400 MHz, DMSO-d₆) δ ppm: 1.40-1.60 (m, 3H), 1.75-1.85 (m, 1H), 1.92-2.00 (m, 2H), 2.21 (s, 4H), 2.60-2.76 (m, 3H), 2.80-3.00 (m, 2H), 3.20-3.30 (m, 1H), 3.35- 3.45 (m, 1H), 3.46-3.56 (m, 1H), 3.72-3.80 (m, 1H), 4.13-4.30 (m, 3H), 4.63-4.83 (m, 2H), 5.15 (s, 2H), 6.64 (s, 1H), 7.20 (d, J = 7.4 Hz, 1H), 7.26-7.35 (m, 3H), 7.36-7.40 (m, 1H), 7.44-7.50 (m, 3H); 1H merged with DMSO residual peak. HPLC: 99.84%. |
| 29 | 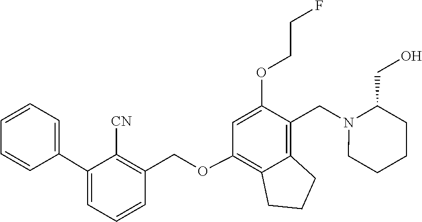 [SMILES: N#Cc1c(COc2cc(OCCF)c(CN3CCCC[C@H]3CO)c3c2CCC3)cccc1-c1ccccc1] | LCMS (ES) m/z = 515.28 [M + H]⁺; ¹H NMR (400 MHz, DMSO-d₆) δ ppm: 1.15-1.30 (m, 3H), 1.32-1.42 (m, 1H), 1.53-1.85 (m, 3H), 1.90- 2.00 (m, 3H), 2.15-2.25 (m, 1H), 2.70-3.00 (m, 4H), 3.18-3.25 (m, 1H), 3.40-3.48 (m, 1H), 3.64-3.76 (m, 1H), 3.85-3.93 (m, 1H), 4.15- 4.32 (m, 3H), 4.63-4.80 (m, 2H), 5.32 (s, 2H), 6.63 (s, 1H), 7.49-7.63 (m, 6H), 7.76 (d, J = 7.2 Hz, 1H), 7.82 (t, J = 7.6 Hz, 1H); HPLC: 97.24%. |
| 30 | 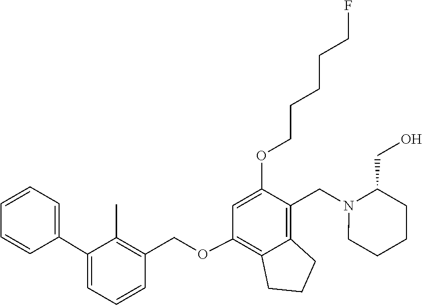 [SMILES: Cc1c(COc2cc(OCCCCCF)c(CN3CCCC[C@H]3CO)c3c2CCC3)cccc1-c1ccccc1] | LCMS (ES) m/z = 546.51 [M + H^{]+}; ¹H NMR (400 MHz, DMSO-d₆) δ ppm: 7.48-7.45 (m, 3H), 7.39-7.39- 7.36 (m, 1H), 7.32-7.26 (m, 3H), 7.19 (d, J = 7.2 Hz, 1H), 6.60 (bs, 1H), 5.17 (bs, 2H), 4.52 (t, J = 6 Hz, 1H), 4.40 (t, J = 6 Hz, 1H), 4.30- 3.81 (m, 4H), 3.70-3.68 (m, 1H), 3.44 (bs, 1H), 3.17 (bs, 1H), 3.05- 2.80 (m, 3H), 2.75 (bs, 2H), 2.55 (bs, 1H), 2.21 (s, 3H), 1.98 (bs, 2H), 1.85-1.48 (m, 9H), 1.38 (bs, 1H), 1.25-1.15 (m, 3H). HPLC: 98.16%. |
| 31 | 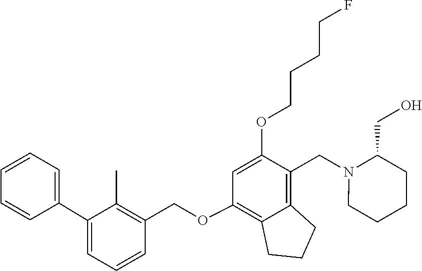 [SMILES: Cc1c(COc2cc(OCCCCF)c(CN3CCCC[C@H]3CO)c3c2CCC3)cccc1-c1ccccc1] | LCMS (ES) m/z = 532.49 [M + H^{]+}; ¹H NMR (400 MHz, DMSO-d₆) δ ppm: 7.48-7.43 (m, 3H), 7.38 (t, J = 7.2 Hz, 1H), 7.32-7.26 (m, 3H), 7.19 (d, J = 6.8 Hz, 1H), 6.59 (s, 1H), 5.14 (s, 2H), 4.57 (t, J = 6.0 Hz, 1H), 4.45 (s, 1H), 4.25 (bs, 1H), 4.02-3.98 (m, 2H), 3.87 (d, J = 11.6 Hz, 1H), 3.72-3.68 (m, 1H), 3.46-3.42 (m, 1H), 3.18 (d, J = 12.8 Hz, 1H), 2.97- 2.90 (m, 1H), 2.86-2.78 (m, 1H), 2.73 (t, J = 7.2 Hz, 2H), 2.21 (s, 4H), 1.99-1.90 (m, 3H), 1.88-1.82 (m, 1H), 1.88-1.77 (m, 3H), 1.67-1.58 (m, 2H), 1.40 (bs, 1H), 1.29-1.23 (m, 4H). HPLC: 99.63%. |
| 32 | 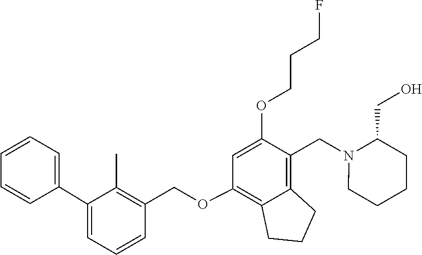 [SMILES: Cc1c(COc2cc(OCCCF)c(CN3CCCC[C@H]3CO)c3c2CCC3)cccc1-c1ccccc1] | LCMS (ES) m/z = 518.47 [M + H^{]+}; ¹H NMR (400 MHz, DMSO-d₆) δ ppm: 7.48-7.43 (m, 3H), 7.37 (t, J = 7.2 Hz, 1H), 7.31 (d, J = 7.2 Hz, 2H), 7.27 (d, J = 7.6 Hz, 1H), 7.19 (d, J = 7.2 Hz, 1H), 6.62 (s, 1H), 5.15 (s, 2H), 4.71 (t, J = 6 Hz, 1H), 4.59 (t, J = 6 Hz, 1H), 4.27 (bs, 1H), 4.12-4.05 (m, 2H), 3.89 (d, J = 12.4 Hz, 1H), 3.72-3.69 (m, 1H), 3.20- 3.11 (m, 1H), 2.96-2.90 (m, 1H), 2.86-2.78 (m, 1H), 2.73 (t, J =7.2 Hz, 2H), 2.21 (s, 4H), 2.16-2.05 (m, 2H), 1.99-1.90 (m, 3H), 1.65 (bs, 1H), 1.59 (bs, 1H), 1.37 bs, 1H), 1.32- 1.28 (m, 4H). 1H merged with |
| | | DMSO residual peak. HPLC: |
| | | 98.52%. |
| 33 | 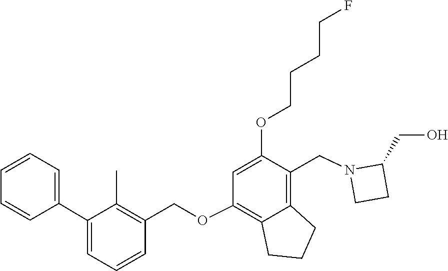 [SMILES: Cc1c(COc2cc(OCCCCF)c(CN3CC[C@H]3CO)c3c2CCC3)cccc1-c1ccccc1] | LCMS (ES) m/z = 504.4 [M + H^{]+}; ¹H NMR (400 MHz, DMSO-d₆) δ ppm: 7.45-7.39 (m, 3H), 7.38-7.34 (m, 1H), 7.34-7.28 (m, 2H), 7.80-7.23 (m, 2H), 6.43 (s, 1H), 5.11 (s, 2H), 4.61 (t, J = 5.6 Hz, 1H), 4.49 (t, J = 5.6 Hz, 1H), 4.09-4.05 (m, 2H), 4.03-3.91 (m, 3H), 3.78-3.60 (m, 3H), 3.49-3.45 (m, 1H), 2.95 (t, J = 7.2 Hz, 2H), 2.88 (t, J = 7.6 Hz, 2H), 2.45-2.38 (m, 1H), 2.26 (s, 3H), 2.13-2.05 (m, 3H), 2.04-1.83 (m, 5H). HPLC: 95.11%. |

### Example 34: Synthesis of 3-(((4-((1-(hydroxymethyl)-2-azabicyclo[4.1.0]heptan-2-yl)methyl)-7-((2-methyl-[1,1′-biphenyl]-3-yl)methoxy)-2,3-dihydro-1H-inden-5-yl)oxy)methyl)benzonitrile

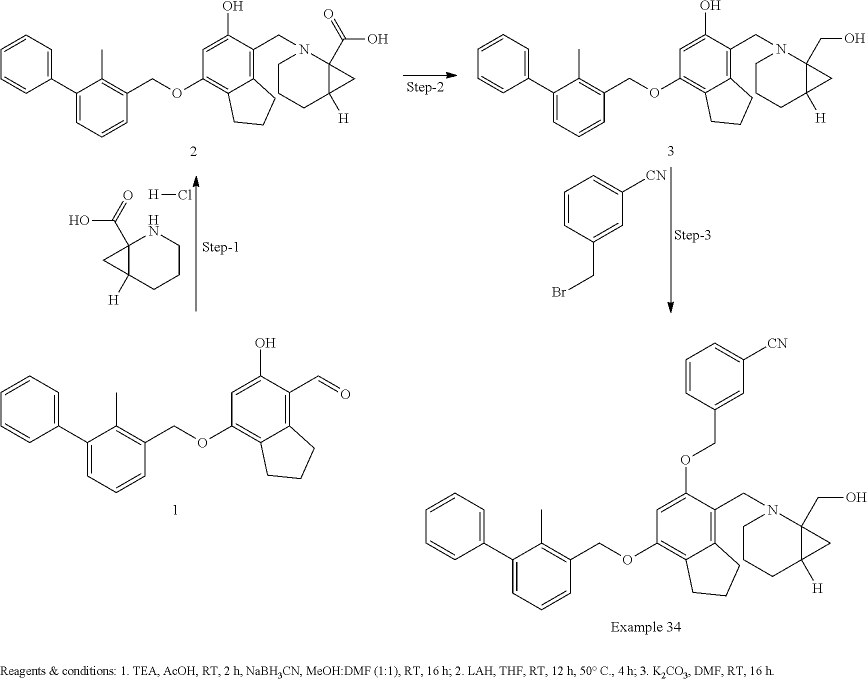

### Step-1: Synthesis of 2-((5-hydroxy-7-((2-methyl-[1,1′-biphenyl]-3-yl)methoxy)-2,3-dihydro-1H-inden-4-yl)methyl)-2-azabicyclo[4.1.0]heptane-1-carboxylic acid (2)

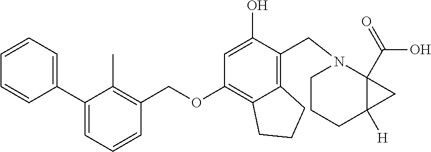 [SMILES: Cc1c(COc2cc(O)c(CN3CCCC4CC43C(=O)O)c3c2CCC3)cccc1-c1ccccc1]

A solution of 5-hydroxy-7-((2-methyl-[1,1′-biphenyl]-3-yl)methoxy)-2,3-dihydro-1H-indene-4-carbaldehyde (1, 0.5 g, 1.39 mmol), 2-azabicyclo [4.1.0]heptane-1-carboxylic acid hydrochloride (0.247 g, 1.67 mmol) in N,N-dimethylformamide (7 mL) and methanol (7 mL), triethylamine (0.282 g, 2.79 mmol) and acetic acid (3 drops) were added and the reaction mixture was stirred for 2 h. To this mixture, sodium cyanoborohydride (0.259 g, 4.18 mmol) was added and continued stirring at room temperature for 16 h. After completion of the reaction, the reaction mixture was diluted with water (10 mL) and extracted with 10% methanol in dichloromethane (2×150 mL). The combined organic layer was dried over sodium sulfate and concentrated. The resulting crude was purified by silica gel flash column chromatography using 0-10% methanol in dichloromethane as eluent to afford the desired product (2, 0.25 g, 37%) as off-white solid. LCMS (ES) m/z=484.49 [M+H]⁺, Crude Purity (79%).

### Step-2: Synthesis of 4-((1-(hydroxymethyl)-2-azabicyclo[4.1.0]heptan-2-yl)methyl)-7-((2-methyl-[1,1′-biphenyl]-3-yl)methoxy)-2,3-dihydro-1H-inden-5-ol (3)

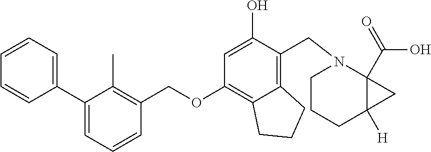 [SMILES: Cc1c(COc2cc(O)c(CN3CCCC4CC43C(=O)O)c3c2CCC3)cccc1-c1ccccc1]

To a solution of 2-((5-hydroxy-7-((2-methyl-[1,1′-biphenyl]-3-yl)methoxy)-2,3-dihydro-1H-inden-4-yl)methyl)-2-azabicyclo[4.1.0]heptane-1-carboxylic acid (2, 0.25 g, 0.51 mmol) in dry tetrahydrofuran (8 mL), lithium aluminum hydride solution 2 M in tetrahydrofuran (10 mL) was added dropwise and the reaction mixture was stirred at room temperature for 12 h followed by heating the mixture at 50° C. for 4 h. After completion of the reaction, the reaction mixture was cooled to 0° C. and ethyl acetate was added dropwise in the reaction mixture. The reaction mixture was then diluted with water (10 mL) and extracted with 10% methanol:dichloromethane (2×100 mL). The combined organic layer was dried over sodium sulfate and concentrated. The resulting crude was purified by silica gel flash column chromatography using 0-30% ethyl acetate in Hexane as eluent to afford the desired product (3, 0.075 g, 30%) as off-white solid. LCMS (ES) m/z=477 [M+H]⁺.

### Step-3: Synthesis of 3-(((4-((1-(hydroxymethyl)-2-azabicyclo[4.1.0]heptan-2-yl)methyl)-7-((2-methyl-[1,1′-biphenyl]-3-yl)methoxy)-2,3-dihydro-1H-inden-5-yl)oxy)methyl)benzonitrile (Example 34)

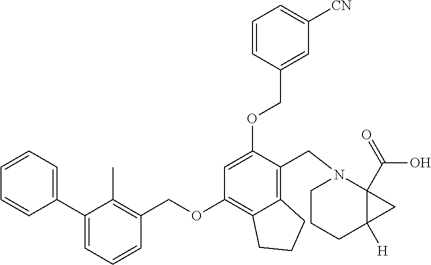 [SMILES: Cc1c(COc2cc(OCc3cccc(C#N)c3)c(CN3CCCC4CC43C(=O)O)c3c2CCC3)cccc1-c1ccccc1]

To a solution of 4-((1-(hydroxymethyl)-2-azabicyclo[4.1.0]heptan-2-yl)methyl)-7-((2-methyl-[1,1′-biphenyl]-3-yl) methoxy)-2,3-dihydro-1H-inden-5-ol (3, 0.075 g, 0.15 mmol) in N,N-dimethylformamide (10 mL), potassium carbonate (0.088 g, 0.63 mmol) and 3-(bromomethyl)benzonitrile (0.062 g, 0.31 mmol) were added. The reaction mixture was stirred at room temperature for 16 h. After completion of the reaction, the reaction mixture was diluted with water (20 mL) and extracted with ethyl acetate (3×20 mL). The combined organic layer was dried over sodium sulfate and concentrated. The resulting crude was purified by silica gel flash column chromatography using 0-50% ethyl acetate in hexane as eluent to afford the title product (Example 34, 0.020 g, 21%) as white solid. LCMS (ES) m/z=585.45 [M+H]⁺; ¹H NMR (400 MHz, DMSO-d₆) δ ppm: 0.40-0.45 (m, 1H), 0.50-0.55 (m, 1H), 1.00-1.12 (m, 2H), 1.50 (bs, 1H), 1.53-1.80 (m, 2H), 1.93-2.03 (m, 2H), 2.14-2.23 (m, 5H), 2.72-2.90 (m, 3H), 3.00-3.13 (m, 2H), 3.48-3.60 (m, 3H), 4.06 (t, J=7.2 Hz, 1H), 5.11 (s, 2H), 5.20 (s, 2H), 6.69 (s, 1H), 7.19 (d, J=7.2 Hz, 1H), 7.26 (t, J=7.6 Hz, 1H), 7.30-7.34 (m, 2H), 7.36-7.48 (m, 4H), 7.60 (t, J=7.6 Hz, 1H), 7.80 (t, J=7.6 Hz, 2H), 7.95 (s, 1H); HPLC purity 98.85%.

The compounds listed in below Table-3 were prepared by a procedure similar to the one described in Example-34 with appropriate variations in reactants, quantity of reagents, protections & deprotections, solvents & reaction conditions. The characterization data of the compounds are summarized herein below table.

TABLE 3

| Example | | |
|---|---|---|
| No. | Structure | Characterization data |
| 35 | 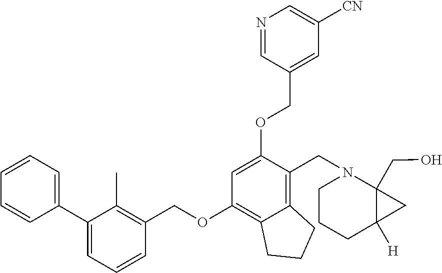 [SMILES: Cc1c(COc2cc(OCc3cncc(C#N)c3)c(CN3CCCC4CC43CO)c3c2CCC3)cccc1-c1ccccc1] | LCMS (ES) m/z = 586.37 [M + H]⁺; ¹H NMR (400 MHz, DMSO-d₆) δ ppm: 0.40-0.43 (m, 1H), 0.52 (t, J = 5.6 Hz, 1H), 1.00-1.12 (m, 2H), 1.13-1.28 (m, 1H), 1.57-1.73 (m, 2H), 1.95-2.01 (m, 2H), 2.17-2.20 (m, 5H), 2.72-2.84 (m, 3H), 3.01- 3.06 (m, 2H), 3.51-3.54 (m, 3H), 4.04 (t, J = 6.0 Hz, 1H), 5.10-5.16 (m, 2H), 5.22-5.30 (m, 2H), 6.74 (s, 1H), 7.19 (d, J = 6.0 Hz, 1H), 7.26 (t, J = 8 Hz, 1H), 7.32 (d, J = 6.8 Hz, 2H), 7.36-7.47 (m, 4H), 8.45 (s, 1H), 8.98 (s, 2H); HPLC: 98.10%. |
| 36 | 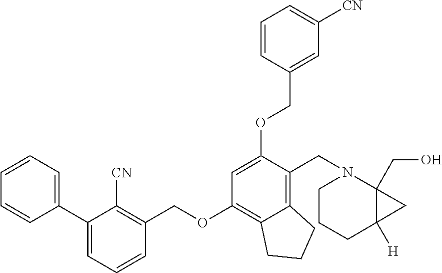 [SMILES: N#Cc1cccc(COc2cc(OCc3cccc(-c4ccccc4)c3C#N)c3c(c2CN2CCCC4CC42CO)CCC3)c1] | LCMS (ES) m/z = 596.27 [M + H]⁺; ¹H NMR (400 MHz, DMSO-d₆) δ ppm: 0.41-0.51 (m, 2H), 1.05-1.09 (m, 2H), 1.23 (bs, 1H), 1.61 (bs, 1H), 1.72 (bs, 1H), 1.96-2.01 (m, 2H), 2.18 (bs, 2H), 2.72-2.90 (m, 3H), 3.00-3.12 (m, 2H), 3.48-3.60 (m, 3H), 4.08 (bs, 1H), 5.19 (s, 2H), 5.30 (s, 2H), 6.68 (bs, 1H), 7.49-7.61 (m, 7H), 7.69 (d, J = 7.6 Hz, 1H), 7.76- 7.80 (m, 3H), 7.92 (bs, 1H); HPLC: 95.18%. |

### Example 37: Synthesis of (S)-5-(((4-((2-(hydroxymethyl)piperidin-1-yl)methyl)-7-((2-methyl-[1,1′-biphenyl]-3-yl)methoxy)-2,3-dihydro-1H-inden-5-yl)oxy)methyl)nicotinamide

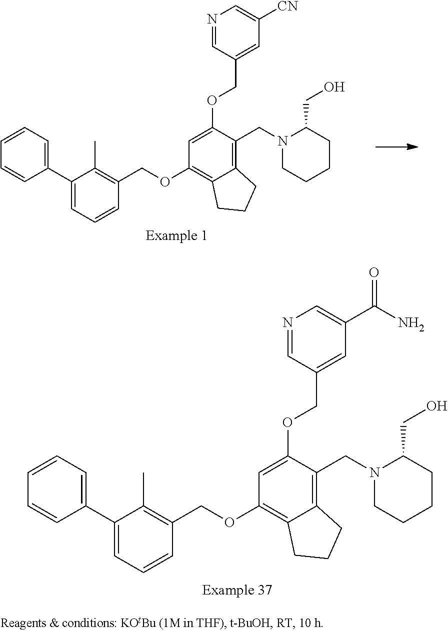 [SMILES: Cc1c(COc2cc(OCc3cncc(C#N)c3)c(CN3CCCC[C@H]3CO)c3c2CCC3)cccc1-c1ccccc1 ; Cc1c(COc2cc(OCc3cncc(C(N)=O)c3)c(CN3CCCC[C@H]3CO)c3c2CCC3)cccc1-c1ccccc1]

To a solution of (S)-5-(((4-((2-(hydroxymethyl)piperidin-1-yl)methyl)-7-((2-methyl-[1,1′-biphenyl]-3-yl)methoxy)-2,3-dihydro-1H-inden-5-yl)oxy)methyl)nicotinonitrile (Example 1, 0.29 g, 0.5 mmol) in tert butanol (10 mL) under nitrogen atmosphere, potassium tert butoxide (1M in tetrahydrofuran, 10 mL) was added and the reaction mixture was stirred at room temperature for 10 h. After completion of the reaction, the reaction mixture was diluted with water (10 mL) and extracted with 10% methanol in dichloromethane (3×55 mL). The organic layer was dried over anhydrous sodium sulfate and concentrated. The resulting crude was purified by silica gel flash column chromatography using 10% methanol in dichloromethane as eluent to afford desired compound. The compound was again purified by reverse phase prep-HPLC (ammonium acetate buffer) to afford the title product (Example 37, 0.030 g, 10%) as white solid. LCMS (ES) m/z=592.22 [M+H]⁺; ¹H NMR (400 MHz, DMSO-d₆) δ ppm: 1.15-1.40 (m, 5H), 1.55-1.65 (m, 2H), 1.88-2.00 (m, 3H), 2.21 (s, 4H), 2.70-3.00 (m, 4H), 3.15-3.22 (m, 1H), 3.35-3.45 (m, 1H), 3.70 (m, 1H), 3.90-3.95 (m, 1H), 4.28 (bs, 1H), 5.13 (s, 2H), 5.20-5.28 (m, 2H), 6.76 (s, 1H), 7.19 (d, J=7.6 Hz, 1H), 7.26 (t, J=7.6 Hz, 1H), 7.30-7.40 (m, 3H), 7.44-7.48 (m, 3H), 7.62 (s, 1H), 8.18 (s, 1H), 8.32 (s, 1H), 8.84 (s, 1H), 8.98 (s, 1H); HPLC purity 98.12%.

### Example 38: Synthesis of (S)-3-(((4-((2-(hydroxymethyl)pyrrolidin-1-yl)methyl)-7-((2-methyl-[1,1′-biphenyl]-3-yl)methoxy)-2,3-dihydro-1H-inden-5-yl)oxy)methyl)benzamide

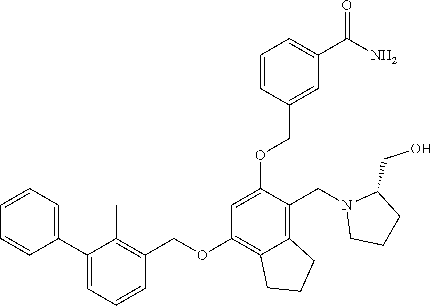 [SMILES: Cc1c(COc2cc(OCc3cccc(C(N)=O)c3)c(CN3CCC[C@H]3CO)c3c2CCC3)cccc1-c1ccccc1]

The Example-38 was prepared by a procedure similar to the one described in Example-37 by using (S)-3-(((4-((2-(hydroxymethyl)pyrrolidin-1-yl)methyl)-7-((2-methyl-[1,1′-biphenyl]-3-yl)methoxy)-2,3-dihydro-1H-inden-5-yl)oxy)methyl)benzonitrile as starting material. LCMS (ES) m/z=577.45 [M+H]⁺; ¹H NMR (400 MHz, DMSO-d₆) δ ppm: 1.70-2.15 (m, 6H), 2.22 (s, 3H), 2.78-2.84 (m, 2H), 2.90-3.40 (m, 6H), 3.60-3.80 (m, 2H), 4.20 (m, 1H), 4.40 (m, 1H), 5.20 (s, 2H), 5.27 (s, 2H), 6.85 (s, 1H), 7.20 (d, J=7.6 Hz, 1H), 7.25-7.33 (m, 3H), 7.36-7.50 (m, 5H), 7.66 (d, J=7.6 Hz, 1H), 7.87 (d, J=7.6 Hz, 1H), 8.04 (m, 2H), 8.63 (bs, 1H); HPLC: 96.9%.

### Example 39: Synthesis of (S)-(1-((5-((1-methyl-1H-pyrazol-4-yl)methoxy)-7-((2-methyl-[1,1′-biphenyl]-3-yl)methoxy)-2,3-dihydro-1H-inden-4-yl)methyl)piperidin-2-yl)methanol

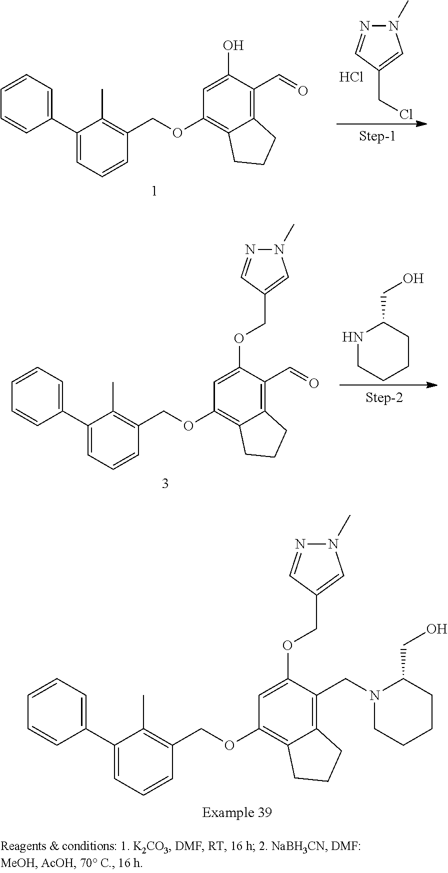

### Step-1: Synthesis of 5-((1-methyl-1H-pyrazol-4-yl)methoxy)-7-((2-methyl-[1,1′-biphenyl]-3-yl)methoxy)-2,3-dihydro-1H-indene-4-carbaldehyde (2)

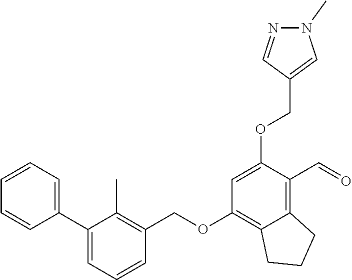 [SMILES: Cc1c(COc2cc(OCc3cnn(C)c3)c(C=O)c3c2CCC3)cccc1-c1ccccc1]

To a stirred solution of 5-hydroxy-7-({2-methyl-[1,1′-biphenyl]-3-yl}methoxy)-2,3-dihydro-1H-indene-4-carbaldehyde (1, 1 g, 2.79 mmol) in N,N-dimethylformamide (20 mL) was added dipotassium carbonate (1.16 g, 3 eq., 8.37 mmol) and 4-(chloromethyl)-1-methyl-1H-pyrazole hydrochloride (0.699 g, 4.18 mmol) at room temperature. The reaction mass was stirred for a further 16 h at the same temperature. After completion, the reaction mixture was diluted with water (20 mL) and extracted with ethyl acetate (3×30 mL). The combined organic layer was dried over sodium sulfate and concentrated. The resulting crude was purified by flash chromatography on silica gel using 40% ethyl acetate in hexane to obtain the desired product (3, 0.8 g, 63.36%) as a yellow solid. LCMS (ES) m/z=453.3 [M+H]⁺.

### Step-2: Synthesis of (S)-(1-((5-((1-methyl-1H-pyrazol-4-yl)methoxy)-7-((2-methyl-[1,1′-biphenyl]-3-yl)methoxy)-2,3-dihydro-1H-inden-4-yl)methyl)piperidin-2-yl)methanol (Example 39)

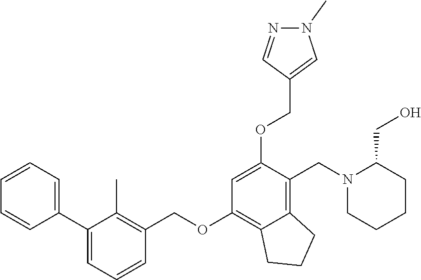 [SMILES: Cc1c(COc2cc(OCc3cnn(C)c3)c(CN3CCCC[C@H]3CO)c3c2CCC3)cccc1-c1ccccc1]

To a stirred solution of 5-[(1-methyl-1H-pyrazol-4-yl)methoxy]-7-({2-methyl-[1,1′-biphenyl]-3-yl}methoxy)-2,3-dihydro-1H-indene-4-carbaldehyde (3, 0.15 g, 3.31 mmol) and [(2S)-piperidin-2-yl]methanol (0.057 g, 14.9 mmol) in dimethylformamide (15 mL) and methanol (15 mL) was added acetic acid (0.95 mL, 16.6 mmol) under nitrogen atmosphere at room temperature and stirred reaction mixture for 6 h at 70° C. To this reaction mass, was added sodium cyanoborohydride (0.625 g, 9.94 mmol) and stirred at the same temperature for further 16 h. After completion of the reaction, monitored by TLC, the reaction mixture was diluted with water (10 mL) and extracted with 10% methanol in dichloromethane (3×15 mL). The organic layer was dried over anhydrous sodium sulfate and concentrated. The resulting crude was purified by silica gel flash column chromatography using 10% methanol in dichloromethane as eluent to get the desired compound. The compound was again purified by reverse-phase prep-HPLC to afford the title product (Example 39, 0.055 g, 30.1%) as a yellow solid. 1H LCMS (ES) m/z=552.21 [M+H]⁺; ¹H NMR (400 MHz, DMSO-d₆) δ ppm: 7.71 (s, 1H), 7.49-7.43 (m, 4H), 7.39-7.35 (m, 1H), 7.33-7.29 (m, 3H), 7.20 (dd, J=7.6, 1.2 Hz, 1H), 6.74 (s, 1H), 5.17 (s, 2H), 4.97 (s, 2H), 4.30 (bs, 1H), 3.85 (bs, 1H), 3.81 (s, 3H), 3.67 (dd, J=10.8, 4.4 Hz, 1H), 3.46 (bs, 1H), 3.20 (bs, 1H), 2.92-2.90 (m, 1H), 2.84-2.78 (m, 1H), 2.74 (t, J=7.2 Hz, 2H), 2.28-2.13 (m, 4H), 1.99-1.93 (m, 2H), 1.91 (s, 2H), 1.70-1.54 (m, 2H), 1.44-1.36 (m, 1H), 1.35-1.17 (m, 3H). HPLC: 95.15%.

The compounds listed in below Table-4 were prepared by a procedure similar to the one described in Example-39 with appropriate variations in reactants, quantity of reagents, protections & deprotections, solvents & reaction conditions. The characterization data of the compounds are summarized herein below table.

TABLE 4

| Example | | |
|---|---|---|
| No. | Structure | Characterization data |
| 40 | 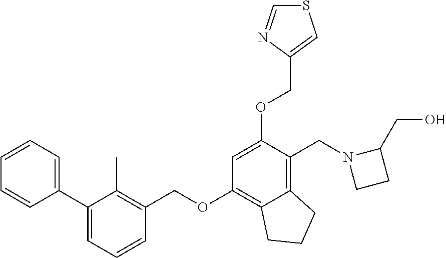 [SMILES: Cc1c(COc2cc(OCc3cscn3)c(CN3CCC3CO)c3c2CCC3)cccc1-c1ccccc1] | LCMS (ES) m/z = 527.25 [M + H]⁺; ¹H NMR (400 MHz, DMSO-d₆) δ ppm: 9.14 (d, J = 20 Hz, 1H), 7.77 (d, J = 1.6 Hz, 1H), 7.46 (t, J = 7.2 Hz, 3H), 7.38 (t, J = 7.2 Hz, 1H), 7.35-7.25 (m, 3H), 7.19 (d, J = 6.8 Hz, 1H), 6.78 (s, 1H), 5.24 (s, 2H), 5.16 (s, 2H), 4.27 (bs, 1H), 3.58- 3.49 (m, 2H), 3.27-3.21 (m, 3H), 3.05-3.01 (m, 1H), 2.95-2.80 (m, 3H), 2.74 (t, J = 7.2 Hz, 2H), 2.22 (s, 3H), 2.01-1.92 (m, 2H), 1.88- 1.78 (m, 1H), 1.76-1.66 (m, 1H); HPLC: 92.96%. |
| 41 | 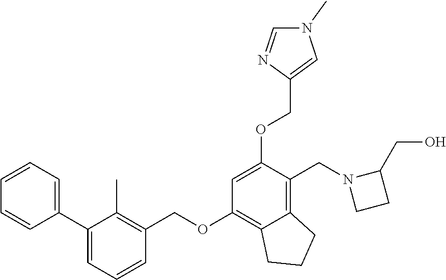 [SMILES: Cc1c(COc2cc(OCc3cn(C)cn3)c(CN3CCC3CO)c3c2CCC3)cccc1-c1ccccc1] | LCMS (ES) m/z = 524.33 [M + H]⁺; ¹H NMR (400 MHz, DMSO-d₆) δ ppm: 9.43 (bs, 1H), 7.58 (bs, 1H), 7.48-7.44 (m, 3H), 7.38 (t, J = 7.6 Hz, 1H), 7.35-7.25 (m, 3H), 7.23 (bs, 1H), 6.92 (s, 1H), 5.24-5.17 (m, 4H), 4.45 (bs, 1H), 4.27 (bs, 2H), 3.88-3.86 (m, 2H), 3.77 (s, 3H), 3.64-3.58 (m, 2H), 2.92 (d, J = 6.8 Hz, 2H), 2.77 (t, J = 7.2 Hz, 2H), 2.22 (s, 3H), 2.01-1.92 (m, 2H); HPLC: 91.08%. |

### Example 42: Synthesis of (1-((7-((2-methyl-[1,1′-biphenyl]-3-yl)methoxy)-5-(pyrimidin-5-ylmethoxy)-2,3-dihydro-1H-inden-4-yl)methyl)azetidin-2-yl)methanol

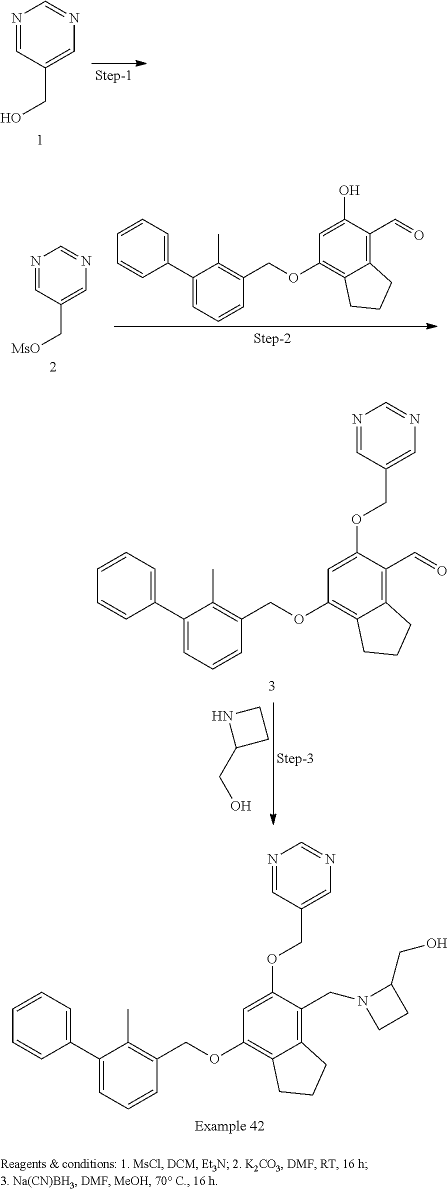

### Step 1: Synthesis of pyrimidin-5-ylmethyl methanesulfonate (2)

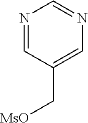 [SMILES: CS(=O)(=O)OCc1cncnc1]

To a solution of (pyrimidin-5-yl)methanol (0.1 g, 0.908 mmol) in dichloromethane (4 mL), triethylamine (0.276 g, 2.72 mmol) and methanesulphonyl chloride (0.171 mL, 1.82 mmol) were sequentially added at 0° C. Progress of the reaction was monitored by TLC. After completion of the reaction, the reaction mixture was diluted with water (40 mL) and extracted with dichloromethane (2×30 mL). The organic layer was dried over anhydrous sodium sulfate and concentrated under vacuum to afford the desired compound (2, 0.12 g crude) which was used in next step without further purification.

### Step 2: Synthesis of 7-((2-methyl-[1,1′-biphenyl]-3-yl)methoxy)-5-(pyrimidin-5-ylmethoxy)-2,3-dihydro-1H-indene-4-carbaldehyde (3)

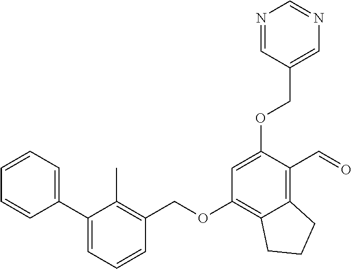 [SMILES: Cc1c(COc2cc(OCc3cncnc3)c(C=O)c3c2CCC3)cccc1-c1ccccc1]

To a solution of 5-hydroxy-7-(1,2,3,4-tetrahydroisoquinoline-2-carbonyl)-2,3-dihydro-1H-indene-4-carbaldehyde (0.57 g, 1.59 mmol) in N,N-dimethylformamide (4 mL), potassium carbonate (0.66 g, 4.78 mmol) and (pyrimidin-5-yl)methyl methanesulfonate (2, 0.3 g, 1.59 mmol) were sequentially added under nitrogen atmosphere at room temperature. The reaction mixture was stirred for 16 h at room temperature. After completion of the reaction (monitored by TLC), the reaction mixture was quenched with chilled water (50 mL) and extracted with ethyl acetate (2×50 mL). The organic layer was dried over anhydrous sodium sulfate and concentrated under reduced pressure. The resulting crude was purified by silica gel flash column chromatography to afford the desired compound (3, 0.16 g, crude) as brown solid. LCMS (ES) m/z=451.35 [M+H]⁺.

### Step 3: Synthesis of (1-((7-((2-methyl-[1,1′-biphenyl]-3-yl) methoxy)-5-(pyrimidin-5-ylmethoxy)-2,3-dihydro-1H-inden-4-yl) methyl) azetidin-2-yl) methanol (Example 42)

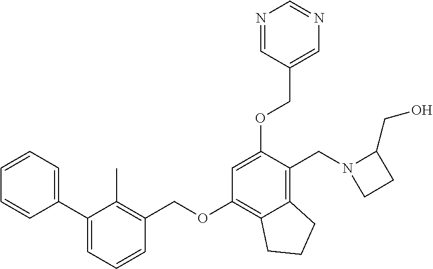 [SMILES: Cc1c(COc2cc(OCc3cncnc3)c(CN3CCC3CO)c3c2CCC3)cccc1-c1ccccc1]

To a solution of 7-((2-methyl-[1,1′-biphenyl]-3-yl) methoxy)-5-(pyrimidin-5-ylmethoxy)-2,3-dihydro-1H-indene-4-carbaldehyde (3, 0.16 g, 0.35 mmol) and azetidin-2-ylmethanol (0.212 g, 1.74 mmol) in dimethylformamide (3 mL) and methanol (7 mL), acetic acid (0.2 mL) was added. The reaction mixture was stirred at 70° C. for 0.5 h and sodium cyanoborohydride (0.059 g, 0.932 mmol) was added to it. The reaction was further stirred at 70° C. for 16 h. After completion of the reaction (monitored by TLC), the reaction mixture was diluted with water (40 mL) and extracted with 10% methanol in dichloromethane (3×30 mL). The organic layer was dried over anhydrous sodium sulfate and concentrated und reduced pressure. The residue was purified by silica gel column chromatography 5% methanol in dichloromethane to afford the title compound (Example 42, 0.008 g, 4.2%) as white solid. LCMS (ES) m/z=522.35 [M+H]⁺; ¹H NMR (400 MHz, DMSO-d₆) δ ppm: 9.17 (s, 1H), 8.95 (s, 2H), 7.46 (t, J=6.8 Hz, 3H), 7.40-7.36 (m, 1H), 7.33-7.31 (m, 2H), 7.26 (t, J=7.6 Hz, 1H), 7.19 (d, J=7.6 Hz, 1H), 6.77 (s, 1H), 5.21 (s, 2H), 5.16 (s, 2H), 4.21 (bs, 1H), 3.53 (d, J=12 Hz, 1H), 3.43 (d, J=12.4 Hz, 1H), 3.21-3.10 (m, 3H), 3.03-2.95 (m, 1H), 2.90-2.80 (m, 2H), 2.79-2.70 (m, 3H), 2.21 (s, 3H), 2.00-1.90 (m, 2H), 1.87-1.80 (m, 1H), 1.75-1.65 (m, 1H). HPLC: 96.47%.

### Example 43: Synthesis of (1-((7-((2-methyl-[1,1′-biphenyl]-3-yl)methoxy)-5-(oxazol-4-ylmethoxy)-2,3-dihydro-1H-inden-4-yl)methyl)azetidin-2-yl)methanol

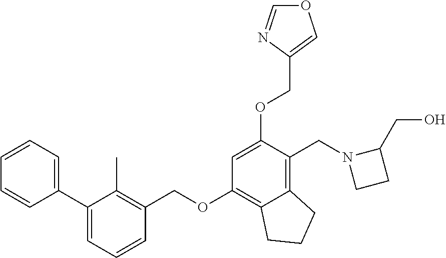 [SMILES: Cc1c(COc2cc(OCc3cocn3)c(CN3CCC3CO)c3c2CCC3)cccc1-c1ccccc1]

The Example-43 was prepared by a procedure similar to the one described in Example-42 by using oxazol-4-ylmethanol as starting material. LCMS (ES) m/z=511.42 [M+H]⁺; ¹H NMR (400 MHz, DMSO-d₆) δ ppm: 8.42 (s, 1H), 8.18 (s, 1H), 7.50-7.42 (M, 3H), 7.40-7.36 (m, 1H), 7.35-7.26 (m, 3H), 7.19 (d, J=6.8 Hz, 1H), 6.79 (s, 1H), 5.17 (s, 2H), 5.03 (s, 2H), 4.24 (bs, 1H), 3.51 (bs, 2H), 3.28-3.18 (m, 3H), 3.02 (bs, 1H), 2.95-2.79 (m, 3H), 2.74 (t, J=7.2 Hz, 2H), 2.22 (s, 3H), 2.00-1.93 (m, 2H), 1.83 (bs, 1H), 1.71 (bs, 1H); HPLC: 92.63%.

### Example 44: Synthesis of (S)-3-cyano-5-(((4-((2-(hydroxymethyl)pyrrolidin-1-yl)methyl)-7-((2-methyl-[1,1′-biphenyl]-3-yl)methoxy)-2,3-dihydro-1H-inden-5-yl)oxy)methyl)pyridine 1-oxide

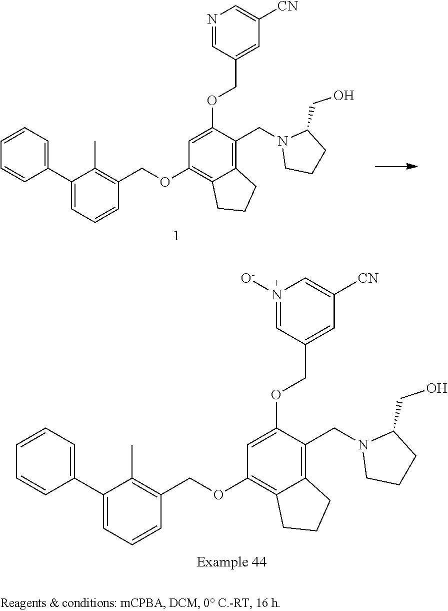

To a stirred solution of 5-{[(4-{[(2S)-2-(hydroxymethyl)pyrrolidin-1-yl]methyl}-7-({2-methyl-[1,1′-biphenyl]-3-yl}methoxy)-2,3-dihydro-1H-inden-5-yl)oxy]methyl}pyridine-3-carbonitrile (1, 0.5 g, 0.89 mmol) in dichloromethane (10 mL) was added 3-chlorobenzene-1-carboperoxoic acid (0.231 mg, 1.34 mmol) at 0° C. and the reaction mixture was stirred for 16 hours at room temperature. After competition of the reaction, the reaction mass was filtered on celite pad and the organic layer was concentrated under reduced pressure and purified by column chromatography to obtain the title compound (Example 44, 0.064 g, 12.4%) as a white solid. LCMS (ES) m/z=576.30 [M+H]⁺; ¹H NMR (400 MHz, DMSO-d₆) δ ppm: 9.01 (dd, J=12, 1.6 Hz, 2H), 8.49 (t, J=1.6 Hz, 1H), 8.46 (bs, 1H), 7.48-7.44 (m, 3H), 7.38 (t, J=7.2 Hz, 1H), 7.33-7.31 (m, 2H), 7.28 (t, J=7.6 Hz, 1H), 7.20 (d, J=6.8 Hz, 1H), 6.85 (s, 1H), 5.27 (s, 2H), 5.21 (s, 2H), 4.48 (d, J=13.2 Hz, 2H), 4.34 (d, J=12.8 Hz, 1H), 4.11 (d, J=11.6 Hz, 1H), 3.39-3.33 (m, 2H), 3.26-3.18 (m, 2H), 2.94-2.90 (m, 2H), 2.77 (t, J=7.2 Hz, 2H), 2.30-2.25 (m, 1H), 2.21 (s, 3H), 2.03-1.91 (m, 3H), 1.87-1.78 (m, 1H), 1.74-1.65 (m, 1H); HPLC: 99.93%.

### Example 45: Synthesis of (S)-3-cyano-5-(((4-((2-(hydroxymethyl)piperidin-1-yl)methyl)-7-((2-methyl-[1,1′-biphenyl]-3-yl)methoxy)-2,3-dihydro-1H-inden-5-yl)oxy)methyl)pyridine 1-oxide

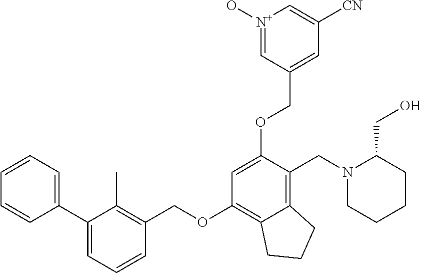

The Example-45 was prepared by a procedure similar to the one described in Example-44 by using (S)-5-(((4-((2-(hydroxymethyl)piperidin-1-yl)methyl)-7-((2-methyl-[1,1′-biphenyl]-3-yl)methoxy)-2,3-dihydro-1H-inden-5-yl)oxy)methyl)nicotinonitrile as starting material. LCMS (ES) m/z=590.35 [M+H]⁺; ¹H NMR (400 MHz, DMSO-d₆) δ ppm: 9.00 (dd, J=8.4, 2.0 Hz, 2H), 8.80 (bs, 1H), 8.47 (s, 1H), 7.48-7.44 (m, 3H), 7.41-7.25 (m, 4H), 7.20 (d, J=6.4 Hz, 1H), 6.82 (s, 1H), 5.29 (s, 2H), 5.20 (s, 2H), 4.59 (d, J=12 Hz, 1H), 4.43 (d, J=12.8 Hz, 2H), 3.47-3.37 (m, 2H), 3.21 (d, J=10.4 Hz, 1H), 2.95-2.83 (m, 2H), 2.76 (t, J=7.2 Hz, 3H), 2.42-2.37 (m, 2H), 2.21 (s, 3H), 2.05-1.78 (m, 3H), 1.65-1.45 (m, 2H), 1.39-1.21 (m, 2H); HPLC: 98.39%.

### Example 46: Synthesis of (S)-5-(((7-((4′-fluoro-2-methyl-[1,1′-biphenyl]-3-yl)methoxy)-4-((2-(hydroxymethyl)piperidin-1-yl)methyl)-2,3-dihydro-1H-inden-5-yl)oxy)methyl)nicotinonitrile

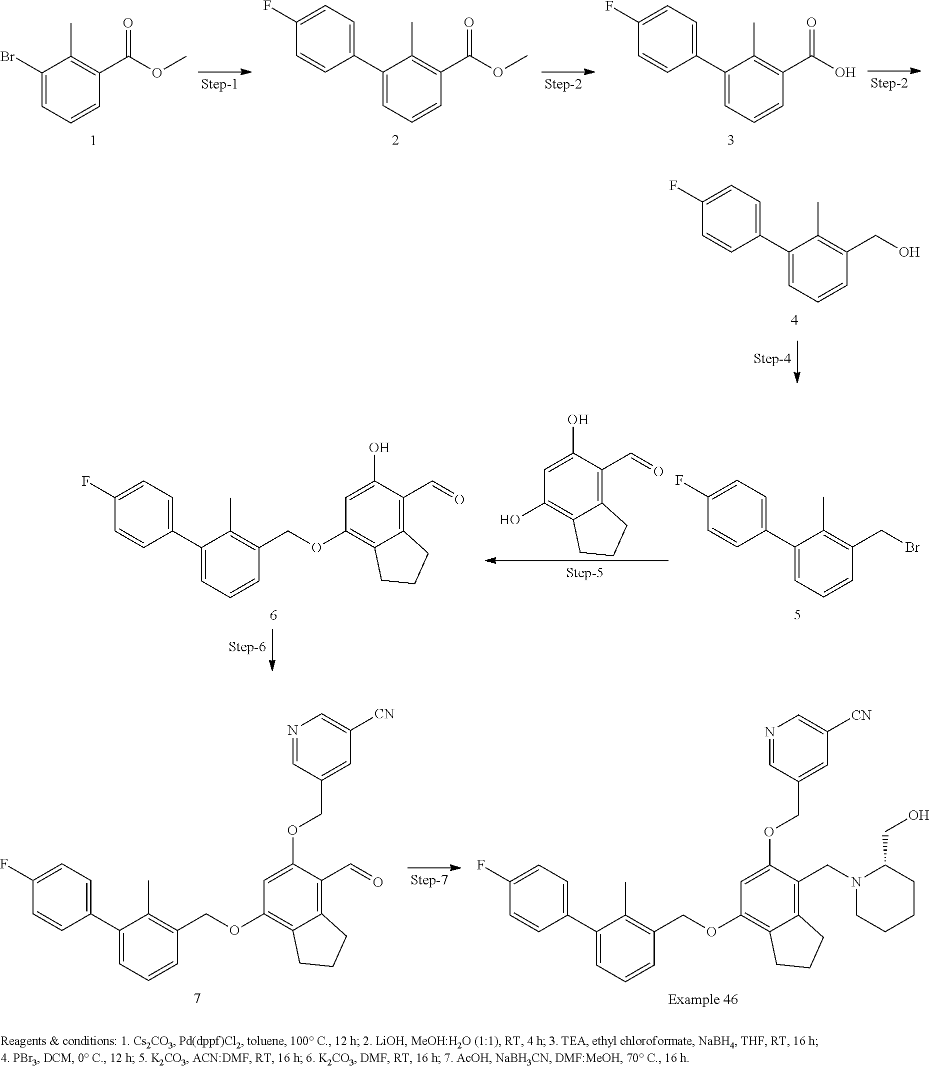

### Step-1: Synthesis of methyl 4′-fluoro-2-methyl-[1,1′-biphenyl]-3-carboxylate (2)

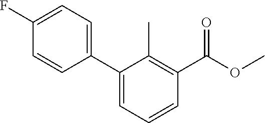 [SMILES: COC(=O)c1cccc(-c2ccc(F)cc2)c1C]

To a stirred solution methyl 3-bromo-2-methylbenzoate (1, 10 g, 43.7 mmol) in toluene (100 mL) was added cesium carbonate (42.7 g, 131 mmol) and (4-fluorophenyl)boronic acid (9.16 g, 65.5 mmol) at room temperature. The reaction mixture was degassed by passing nitrogen gas through reaction mass, and was then added Pd(dppf)Cl₂ (3.19 g, 4.37 mmol). The resulting reaction mixture was stirred for 12 h at 100° C. After completion of the reaction, monitored by TLC, water (50 mL) was added and extracted with ethyl acetate. The combined organic layer was concentrated under reduced pressure and purified by silica gel column chromatography to obtain the desired product (2, 10.2 g, 95% yield) as white solid. LCMS (ES) m/z=245.2 [M+H]⁺.

### Step-2: Synthesis of 4′-fluoro-2-methyl-[1,1′-biphenyl]-3-carboxylic acid (3)

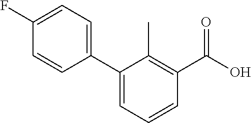 [SMILES: Cc1c(C(=O)O)cccc1-c1ccc(F)cc1]

To a stirred solution of methyl 4′-fluoro-2-methyl-[1,1′-biphenyl]-3-carboxylate (2, 5 g, 20.5 mmol) in methanol (10 mL) and water (10 mL) was added lithium hydroxide (4.9 g, 205 mmol) at room temperature and stirred for 4 hours. The reaction mass was acidified to pH 2 using 2M hydrochloric acid solution and then extracted with ethyl acetate. The organic layer was concentrated under the reduced pressure and the crude was purified by flash silica gel column chromatography to get desired product (3, 5.2 g, 87%) as white solid. LCMS (ES) m/z=231.3 [M+H]⁺.

### Step-3: Synthesis of {4′-fluoro-2-methyl-[1,1′-biphenyl]-3-yl}methanol (4)

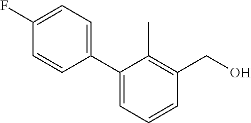 [SMILES: Cc1c(CO)cccc1-c1ccc(F)cc1]

To a stirred solution of 4′-fluoro-2-methyl-[1,1′-biphenyl]-3-carboxylic acid (3, 2.2 g, 9.56 mmol) in tetrahydrofuran (44 mL) was added triethylamine (2.66 mL, 19.1 mmol) at room temperature. The reaction mass was cooled to 0° C. and added ethyl chloroformate (1 mL, 10.5 mmol) over a period of 10 min. After stirring the reaction mixture for 2 h at 0° C., sodium borohydride (1.08 g, 28.7 mmol) was added portion wise and stirred for 16 h. The reaction was quenched by addition of water (20 mL) and extracted with ethyl acetate (2×50 mL). The organic layer was dried over sodium sulphate, concentrated under reduced pressure and the crude was purified by silica gel column chromatography to obtain the desired product (4, 1.5 g, 73%) as colorless oil. LCMS (ES) m/z=217.2 [M+H]⁺.

### Step-4: Synthesis of 3-(bromomethyl)-4′-fluoro-2-methyl-1,1′-biphenyl (5)

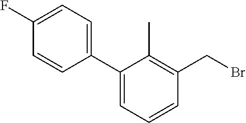 [SMILES: Cc1c(CBr)cccc1-c1ccc(F)cc1]

To a stirred solution of {4′-fluoro-2-methyl-[1,1′-biphenyl]-3-yl}methanol (4, 950 mg, 4.39 mmol) in dichloromethane (15 mL) was added tribromophosphane (0.46 mL, 4.83 mmol) at 0° C. under nitrogen atmosphere. The resulting solution was stirred for further 2 h. The reaction was quenched with aqueous sodium bicarbonate (10 mL) solution. The organic layer was separated, dried over sodium sulphate and concentrated under reduced pressure to obtain the desired product (5, 1.2 g, 97%) as white solid. LCMS (ES) m/z=280.1 [M+H]⁺.

### Step-5: Synthesis of 7-({4′-fluoro-2-methyl-[1,1′-biphenyl]-3-yl}methyl)-5-hydroxy-2,3-dihydro-1H-indene-4-carbaldehyde (6)

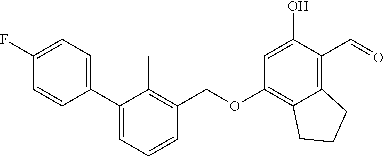 [SMILES: Cc1c(COc2cc(O)c(C=O)c3c2CCC3)cccc1-c1ccc(F)cc1]

To a stirred solution of 5,7-dihydroxy-2,3-dihydro-1H-indene-4-carbaldehyde (0.6 g, 3.37 mmol) in acetonitrile (20 mL) and N,N-dimethylformamide (10 mL) was added dipotassium carbonate (1.4 g, 10.1 mmol) and 3-(bromomethyl)-4′-fluoro-2-methyl-1,1′-biphenyl (5, 940 mg, 3.37 mmol) at room temperature and stirred the reaction mixture for 16 h at room temperature. After completion of the reaction as monitored by TLC, the solvent was evaporated, diluted with water (30 mL) and extracted with ethyl acetate (2×30 mL). The organic layer was dried over anhydrous sodium sulfate and concentrated. The resulting crude was purified by silica gel flash column chromatography to get desired product (6, 0.5 g, 40%) as brown solid. LCMS (ES) m/z=377.1 [M+H]⁺.

### Step-6: Synthesis of 5-({[7-({4′-fluoro-2-methyl-[1,1′-biphenyl]-3-yl}methoxy)-4-formyl-2,3-dihydro-1H-inden-5-yl]oxy}methyl)pyridine-3-carbonitrile (7)

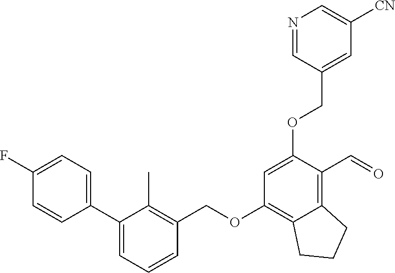 [SMILES: Cc1c(COc2cc(OCc3cncc(C#N)c3)c(C=O)c3c2CCC3)cccc1-c1ccc(F)cc1]

To a stirred solution of 7-({4′-fluoro-2-methyl-[1,1′-biphenyl]-3-yl}methoxy)-5-hydroxy-2,3-dihydro-1H-indene-4-carbaldehyde (6, 0.38 g, 1.01 mmol) in N,N-dimethylformamide (10 mL) was added dipotassium carbonate (0.698 g, 5.05 mmol) and (5-cyanopyridin-3-yl)methyl methanesulfonate (0.428 g, 2.02 mmol) at room temperature. The reaction mass was stirred for further 16 h at the same temperature. After completion, the reaction mixture was diluted with water (20 mL) and extracted with ethyl acetate (3×30 mL). The combined organic layer was dried over sodium sulfate and concentrated. The resulting crude was purified by flash chromatography on silica gel using ethyl acetate in hexane to obtain the desired product (7, 0.3 g, 60.33%) as a yellow solid. LCMS (ES) m/z=493.5 [M+H]⁺

### Step-7: Synthesis of 5-({[7-({4′-fluoro-2-methyl-[1,1′-biphenyl]-3-yl}methoxy)-4-{[(2S)-2-(hydroxymethyl)piperidin-1-yl]methyl}-2,3-dihydro-1H-inden-5-yl]oxy}methyl)pyridine-3-carbonitrile (Example 46)

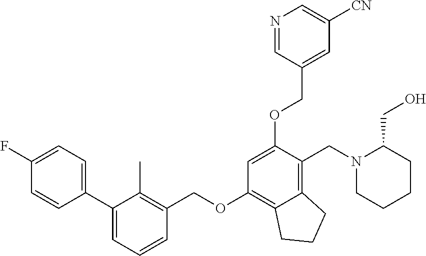 [SMILES: Cc1c(COc2cc(OCc3cncc(C#N)c3)c(CN3CCCC[C@H]3CO)c3c2CCC3)cccc1-c1ccc(F)cc1]

To a stirred solution of 5-({[7-({4′-fluoro-2-methyl-[1,1′-biphenyl]-3-yl}methoxy)-4-formyl-2,3-dihydro-1H-inden-5-yl]oxy}methyl)pyridine-3-carbonitrile (7, 0.650 g, 1.32 mmol) and [(2S)-piperidin-2-yl]methanol (0.228 g, 1.98 mmol) in N,N-dimethylformamide (5 mL) and methanol (5 mL) was added acetic acid (0.396 g, 6.6 mmol) under nitrogen atmosphere at room temperature and stirred reaction mixture for 6 h at 70° C. To this reaction mass, was added sodium cyanoborohydride (0.249 g, 3.96 mmol) and stirred at the same temperature for a further 16 h. After completion of the reaction as monitored by TLC, the reaction mixture was diluted with water (10 mL) and extracted with 10% methanol in dichloromethane (3×15 mL). The organic layer was dried over anhydrous sodium sulfate and concentrated. The resulting crude was purified by silica gel flash column chromatography using 10% methanol in dichloromethane as eluent to get the desired compound. The compound was again purified by reverse-phase prep-HPLC to afford the title compound (20 mg, 33.8 μmol) (Example 46, 0.020 g, 2.56%) as white solid. LCMS (ES) m/z=592.35 [M+H]⁺; ¹H NMR (400 MHz, DMSO-d₆) δ ppm: 9.00 (dd, J=14.4, 2.0 Hz, 2H), 8.41 (s, 1H), 7.44 (d, J=7.2 Hz, 1H), 7.38-7.34 (m, 2H), 7.30-7.24 (m, 3H), 7.18 (d, J=6.8 Hz, 1H), 6.73 (s, 1H), 5.32-5.23 (m, 2H), 5.13 (s, 2H), 4.29 (bs, 1H), 3.98 (d, J=12.4 Hz, 1H), 3.69 (d, J=10.4 Hz, 1H), 3.41 (bs, 1H), 3.14 (d, J=12.0 Hz, 1H), 2.99-2.80 (m, 2H), 2.73 (t, J=7.2 Hz, 2H), 2.52 (bs, 1H), 2.20 (bs, 4H), 1.98-1.86 (m, 3H), 1.66-1.60 (m, 2H), 1.39 (bs, 1H), 1.31-1.25 (m, 3H). HPLC: 95.58%.

The compounds listed in below Table-5 were prepared by a procedure similar to the one described in Example-46 with appropriate variations in reactants, quantity of reagents, protections & deprotections, solvents & reaction conditions. The characterization data of the compounds are summarized herein below table.

TABLE 5

| Example | | |
|---|---|---|
| No. | Structure | Characterization data |
| 47 | 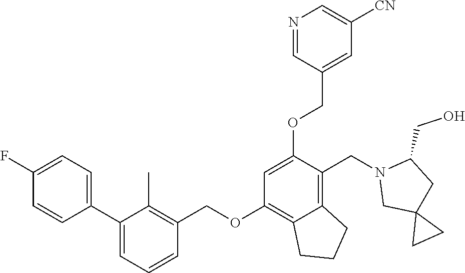 [SMILES: Cc1c(COc2cc(OCc3cncc(C#N)c3)c(CN3CC4(CC4)C[C@H]3CO)c3c2CCC3)cccc1-c1ccc(F)cc1] | LCMS (ES) m/z = 604.35 [M + H]⁺; ¹H NMR (400 MHz, DMSO-d₆) δ ppm: 8.99 (dd, J = 4.8, 2.0 Hz, 2H), 8.41 (t, J = 2.0 Hz, 1H), 7.45 (d, J = 6.8 Hz, 1H), 7.38-7.31 (m, 2H), 7.30-7.24 (m, 3H), 7.19 (d, J = 6.4 Hz, 1H), 6.73 (s, 1H), 5.28-5.21 (m, 2H), 5.13 (s, 2H), 3.84 (d, J = 12.0 Hz, 1H), 3.51 (dd, J = 10.4, 4.4 Hz, 1H), 3.36-3.25 (m, 1H), 3.03-2.91 (m, 1H), 2.89-2.80 (m, 1H), 2.74 (t, J = 7.2 Hz, 3H), 2.44 (bs, 1H), 2.33- 2.31 (m, 1H), 2.20 (s, 3H), 2.02-1.92 (m, 2H), 1.90-1.85 (m, 3H), 1.51- 1.47 (m, 1H), 0.45-0.34 (m, 4H); HPLC: 98.51%. |
| 48 | 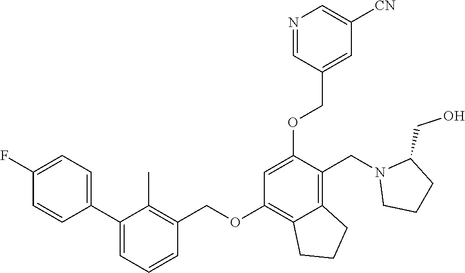 [SMILES: Cc1c(COc2cc(OCc3cncc(C#N)c3)c(CN3CCC[C@H]3CO)c3c2CCC3)cccc1-c1ccc(F)cc1] | LCMS (ES) m/z = 578.35 [M + H]⁺; ¹H NMR (400 MHz, DMSO-d₆) δ ppm: 8.99 (s, 2H), 8.43 (s, 1H), 7.44 (d, J = 7.2 Hz, 1H), 7.38-7.34 (m, 2H), 7.30-7.24 (m, 3H), 7.18 (d, J = 7.6 Hz, 1H), 6.74 (s, 1H), 5.31-5.22 (m, 2H), 5.13 (s, 2H), 4.21 (s, 1H), 3.82 (d, J = 12.0 Hz, 1H), 3.43-3.37 (m, 2H), 3.20-3.13 (m, 1H), 2.94- 2.81 (m, 2H), 2.74 (t, J = 6.8 Hz, 2H), 2.65 (bs, 2H), 2.20 (bs, 4H), 2.00-1.92 (m, 2H), 1.83-1.81 (m, 1H), 1.60-1.47 (m, 3H); HPLC: 98.26%. |

### Example 49: Synthesis of 5-((5-cyanopyridin-3-yl)methoxy)-7-((2-methyl-[1,1′-biphenyl]-3-yl)methoxy)-N-(1-methylpiperidin-4-yl)-2,3-dihydro-1H-indene-4-carboxamide

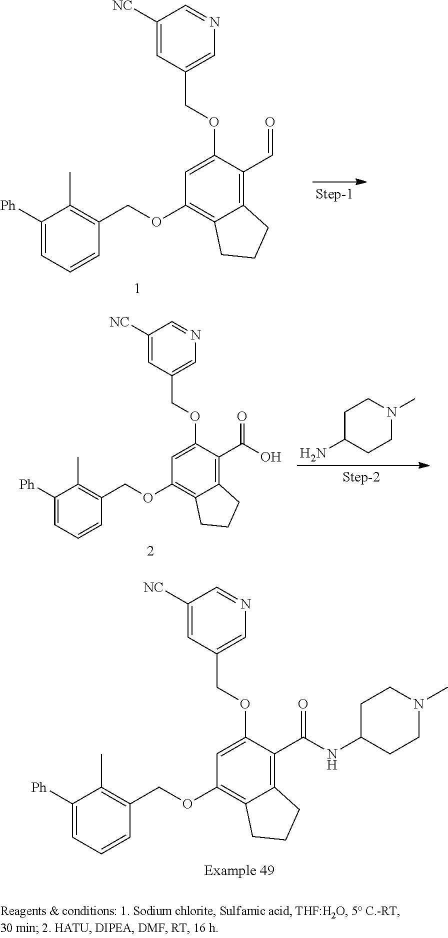

### Step-1: Synthesis of 5-((5-cyanopyridin-3-yl)methoxy)-7-((2-methyl-[1,1′-biphenyl]-3-yl)methoxy)-2,3-dihydro-1H-indene-4-carboxylic acid (2)

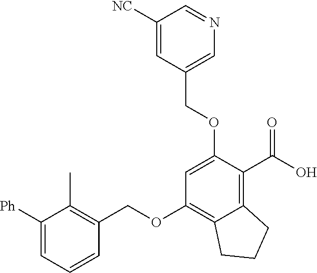 [SMILES: Cc1c(COc2cc(OCc3cncc(C#N)c3)c(C(=O)O)c3c2CCC3)cccc1-c1ccccc1]

To a stirred solution of 5-({[4-formyl-7-({2-methyl-[1,1′-biphenyl]-3-yl}methoxy)-2,3-dihydro-1H-inden-5-yl]oxy}methyl)pyridine-3-carbonitrile (1, 1 g, 2.11 mmol) in tetrahydrofuran (20 mL) and water (7 mL) was added sodium chlorite (0.572 g, 6.32 mmol) and sulfamic acid (0.614 g, 6.32 mmol) at 5° C. The reaction mixture was stirred at 5° C. for 10 minutes and then room temperature for 20 minutes. The reaction mixture was diluted with ethyl acetate (20 mL) and washed with water (20 mL). The precipitate was collected by filtration to give desired product (2, 0.850 g, 82%) as off white solid. LCMS (ES) m/z=491.2 [M+H]⁺

### Step-2: Synthesis of 5-[(5-cyanopyridin-3-yl)methoxy]-7-({2-methyl-[1,1′-biphenyl]-3-yl}methoxy)-N-(1-methylpiperidin-4-yl)-2,3-dihydro-1H-indene-4-carboxamide (Example 49)

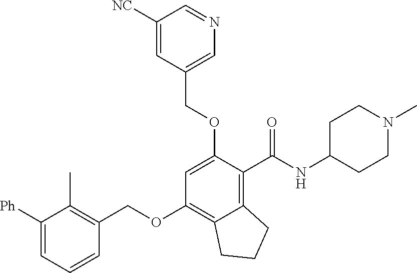 [SMILES: Cc1c(COc2cc(OCc3cncc(C#N)c3)c(C(=O)NC3CCN(C)CC3)c3c2CCC3)cccc1-c1ccccc1]

To a stirred solution of 5-[(5-cyanopyridin-3-yl)methoxy]-7-({2-methyl-[1,1′-biphenyl]-3-yl}methoxy)-2,3-dihydro-1H-indene-4-carboxylic acid (2, 0.350 g, 0.713 mmol) and 4-azaniumyl-1-methylpiperidin-1-ium (0.166 g, 1.43 mmol) in N,N-dimethylformamide (17.5 mL) was added and hexafluoro-λ⁵-phosphanuide 1-[bis(dimethylamino)methylidene]-1H-1λ⁵-[1,2,3]triazolo[4,5-b]pyridin-3-ium-1-ylium-3-olate (0.543 g, 1.43 mmol) and ethylbis(propan-2-yl)amine (0.32 mL, 1.78 mmol) at room temperature. The reaction mixture was stirred for 16 h at room temperature and monitored by LC-MS. After completion of the reaction the reaction mixture was quenched with ice cold water (15 mL). The precipitate was collected by filtration to give title compound (Example 49, 0.15 g, 35.83%) as white solid. LCMS (ES) m/z=587.35 [M+H]⁺; ¹H NMR (400 MHz, DMSO-d₆) δ ppm: 9.00 (d, J=1.6 Hz, 1H), 8.95 (d, J=2.0 Hz, 1H), 8.37 (t, J=2.0 Hz, 1H), 7.96 (d, J=8.0 Hz, 1H), 7.47-7.44 (m, 3H), 7.39-7.36 (m, 1H), 7.33-7.30 (m, 2H), 7.27 (t, J=7.6 Hz, 1H), 7.20 (d, J=6.4 Hz, 1H), 6.81 (s, 1H), 5.25 (s, 2H), 5.21 (s, 2H), 3.69-3.62 (m, 1H), 2.81 (t, J=7.2 Hz, 2H), 2.75 (t, J=7.2 Hz, 2H), 2.69-2.66 (m, 2H), 2.21 (s, 3H), 2.13 (s, 3H), 2.01-1.89 (m, 4H), 1.68 (d, J=12 Hz, 2H), 1.46-1.37 (m, 2H). HPLC: 99.04%.

The compounds listed in below Table-6 were prepared by a procedure similar to the one described in Example-49 with appropriate variations in reactants, quantity of reagents, protections & deprotections, solvents & reaction conditions. The characterization data of the compounds are summarized herein below table.

TABLE 6

| Example | | |
|---|---|---|
| No. | Structure | Characterization data |
| 50 | 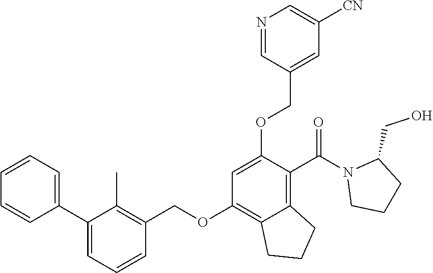 [SMILES: Cc1c(COc2cc(OCc3cncc(C#N)c3)c(C(=O)N3CCC[C@H]3CO)c3c2CCC3)cccc1-c1ccccc1] | LCMS (ES) m/z = 574.35 [M + H]⁺; ¹H NMR (400 MHz, DMSO-d₆) δ ppm: 9.0-8.97 (m, 1H), 8.91-8.88 (m, 1H), 8.31-8.25 (m, 1H), 7.50- 7.44 (m, 3H), 7.38 (t, J = 7.2 Hz, 1H), 7.34-7.24 (m, 3H), 7.22-7.19 (m, 1H), 6.86-6.82 (m, 1H), 5.39- 5.14 (m, 4H), 4.84-4.66 (m, 1H), 4.08-4.06 (m, 1H), 3.67-3.60 (m, 1H), 3.63-3.60 (m, 1H), 3.56-3.35 (m, 1H), 3.19-3.08 (m, 1H), 3.06- 2.95 (m, 1H), 2.85-2.55 (m, 4H), 2.22 (d, J = 6.0 Hz, 3H), 2.06-1.60 (m, 5H); HPLC: 98.63%. |
| 51 | 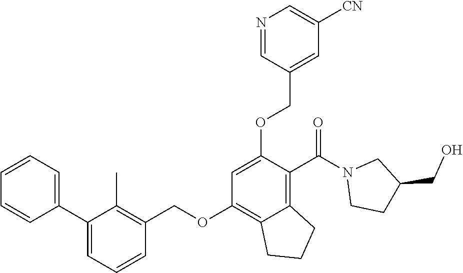 [SMILES: Cc1c(COc2cc(OCc3cncc(C#N)c3)c(C(=O)N3CC[C@H](CO)C3)c3c2CCC3)cccc1-c1ccccc1] | LCMS (ES) m/z = 574.30 [M + H]⁺; ¹H NMR (400 MHz, DMSO-d₆) δ ppm: 8.99 (s, 2H), 8.89 (s, 1H), 8.27 (d, J = 1.6 Hz, 1H), 7.48-7.44 (m, 3H), 7.40-7.36 (m, 1H), 7.32 (d, J = 7.2 Hz, 2H), 7.28 (t, J = 7.6 Hz, 1H), 7.20 (d, J = 7.2 Hz, 1H), 6.83 (s, 1H), 5.31-5.29 (m, 2H), 5.19 (s, 2H), 4.72-4.57 (m, 1H), 3.57-3.47 (m, 1H), 3.44-3.38 (m, 1H), 3.29-3.03 (m, 3H), 2.98-2.82 (m, 1H), 2.80- 2.70 (m, 3H), 2.69-2.58 (m, 1H), 2.35-2.14 (m, 4H), 2.05-1.78 (m, 3H), 1.69-1.49 (m, 1H); HPLC: 99.5%. |

### Example 52: Synthesis of N-((1-((5-((5-cyanopyridin-3-yl)methoxy)-7-((2-methyl-[1,1′-biphenyl]-3-yl)methoxy)-2,3-dihydro-1H-inden-4-yl)methyl)pyrrolidin-2-yl)methyl)acetamide

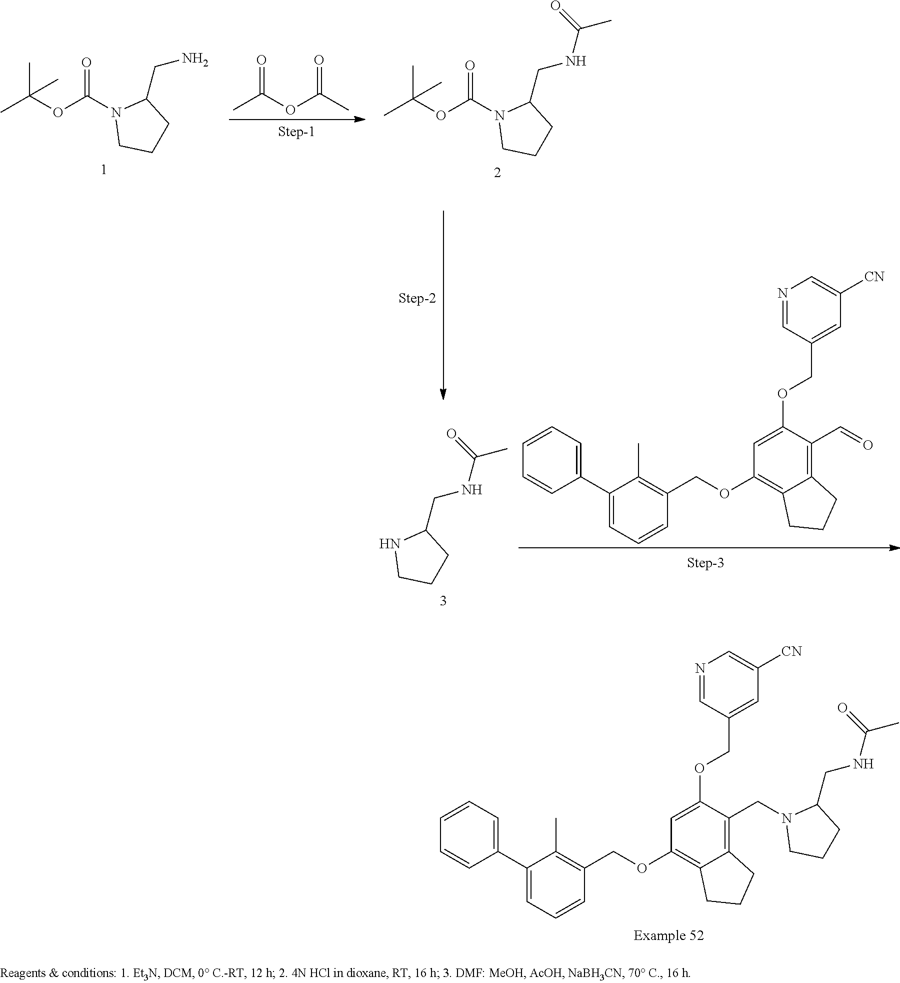

### Step-1: Synthesis of tert-butyl 2-(acetamidomethyl)pyrrolidine-1-carboxylate (2)

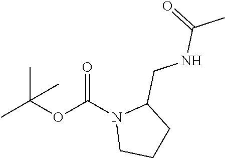 [SMILES: CC(=O)NCC1CCCN1C(=O)OC(C)(C)C]

To a stirred solution of tert-butyl 2-(aminomethyl)pyrrolidine-1-carboxylate (1, 0.5 g, 2.5 mmol) in dichloromethane (10 mL) was added triethylamine (0.69 mL, 4.99 mmol) and acetyl acetate (0.382 mg, 3.74 mmol) at 0° C. The reaction mixture was stirred for 12 h at room temperature. The crude product was quenched with ice cold water and extracted with dichloromethane. The organic layer was dried over sodium sulfate, concentrated under reduced pressure and purified the crude product by silica-gel column chromatography using ethyl acetate in hexane to afford the desired product (2, 0.58 g, 95.8% yield) as colorless oil. LCMS (ES) m/z=243.2 [M+H]⁺

### Step-2: Synthesis of N-(pyrrolidin-2-ylmethyl)acetamide (3)

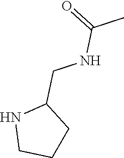

A solution of tert-butyl 2-(acetamidomethyl)pyrrolidine-1-carboxylate (2, 0.58 g, 2.39 mmol) in 4N hydrochloric acid in dioxane (15 mL) was stirred for 16 h at room temperature. The solvent was evaporated under reduced pressure to get the desired product (3, 0.32 g, 94.02% yield) as a colorless oil. LCMS (ES) m/z=143.1 [M+H]⁺

### Step-3: Synthesis of N-((1-((5-((5-cyanopyridin-3-yl)methoxy)-7-((2-methyl-[1,1′-biphenyl]-3-yl)methoxy)-2,3-dihydro-1H-inden-4-yl)methyl)pyrrolidin-2-yl)methyl)acetamide (Example 52)

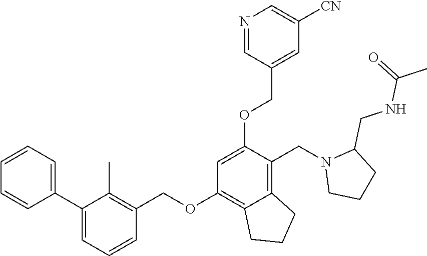 [SMILES: CC(=O)NCC1CCCN1Cc1c(OCc2cncc(C#N)c2)cc(OCc2cccc(-c3ccccc3)c2C)c2c1CCC2]

To a stirred solution of 5-({[4-formyl-7-({2-methyl-[1,1′-biphenyl]-3-yl}methoxy)-2,3-dihydro-1H-inden-5-yl]oxy}methyl)pyridine-3-carbonitrile (0.5 g, 1.05 mmol) and N-[(pyrrolidin-2-yl)methyl]acetamide (3, 0.225 g, 1.58 mmol) in N,N-dimethylformamide (5 mL) and methanol (5 mL) was added acetic acid (0.18 mL, 3.16 mmol) under nitrogen atmosphere at room temperature and the reaction mixture was stirred for 6 h at 70° C. To this reaction mass, was added sodium cyano borohydride (199 mg, 3.16 mmol) and stirred at the same temperature for a further 16 h. After completion of the reaction as monitored by TLC, the reaction mixture was diluted with water (10 mL) and extracted with 10% methanol in dichloromethane (3×15 mL). The combined organic layer was dried over anhydrous sodium sulfate and concentrated. The resulting crude was purified by silica gel flash column chromatography using 10% methanol in dichloromethane as eluent to get the desired compound. The compound was again purified by reverse-phase prep-HPLC to afford the title product (41 mg, 68.2 μmol) (Example 52, 0.041 g, 6.48%) as white solid. LCMS (ES) m/z=601.40 [M+H]⁺. ¹H NMR (400 MHz, DMSO-d₆) δ ppm: 8.98 (d, J=2.0 Hz, 2H), 8.42 (s, 1H), 7.49-7.43 (m, 4H), 7.40-7.36 (m, 1H), 7.33-7.31 (m, 2H), 7.26 (t, J=7.6 Hz, 1H), 7.19 (d, J=7.6 Hz, 1H), 6.75 (s, 1H), 5.32-5.23 (m, 2H), 5.14 (s, 2H), 3.78 (d, J=12.4 Hz, 1H), 3.35-3.29 (m, 2H), 2.97-2.81 (m, 3H), 2.80-2.68 (m, 4H), 2.21 (s, 3H), 2.20-2.12 (m, 1H), 2.03-1.94 (m, 2H), 1.76 (s, 4H), 1.60-1.40 (m, 3H). HPLC purity 87.59%.

### Example 53: Synthesis of 5-(((4-((2-(aminomethyl)pyrrolidin-1-yl)methyl)-7-((2-methyl-[1,1′-biphenyl]-3-yl)methoxy)-2,3-dihydro-1H-inden-5-yl)oxy)methyl)nicotinonitrile

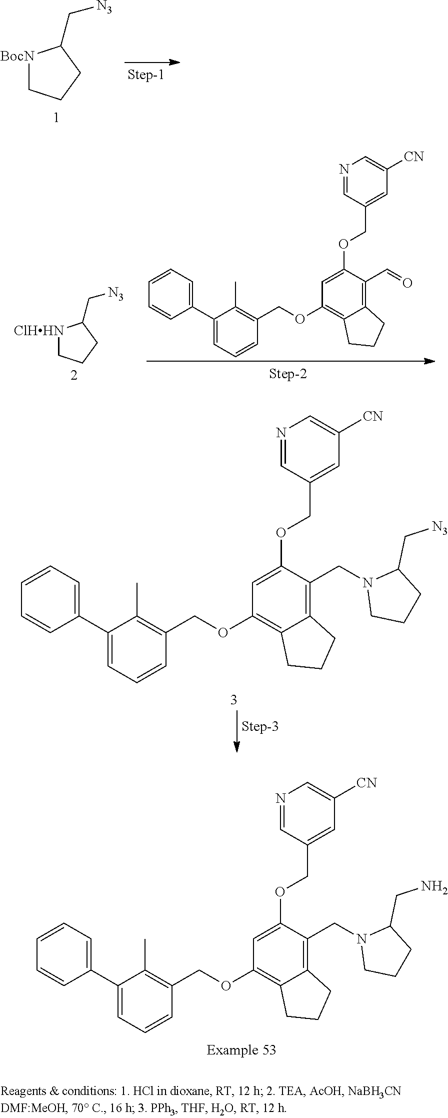

### Step-1: Synthesis of 2-(azidomethyl)pyrrolidine hydrochloride (2)

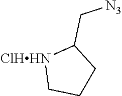

To a stirred solution of tert-butyl 2-(azidomethyl)pyrrolidine-1-carboxylate (1, 1.5 g, 6.63 mmol) in dioxane (10 mL) was added hydrochloride in dioxane (12 M) solution at 0° C. and stirred the reaction mixture for further 12 h at room temperature. The solvent was removed under reduced pressure to get the desired product (2, 0.7 g, crude) as hydrochloric acid salt. The crude material was as such used in the next step.

### Step-2: Synthesis of 5-{[(4-[2-(azidomethyl)pyrrolidin-1-yl]methyl}-7-({2-methyl-[1,1′-biphenyl]-3-yl}methoxy)-2,3-dihydro-1H-inden-5-yl)oxy]methyl}pyridine-3-carbonitrile (3)

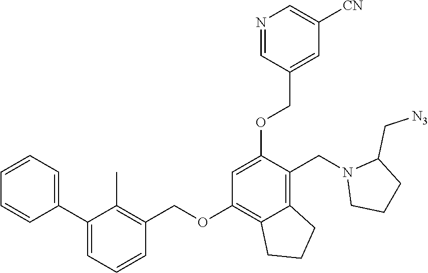 [SMILES: Cc1c(COc2cc(OCc3cncc(C#N)c3)c(CN3CCCC3CN=[N+]=[N-])c3c2CCC3)cccc1-c1ccccc1]

To a stirred solution of 5-({[4-formyl-7-({2-methyl-[1,1′-biphenyl]-3-yl}methoxy)-2,3-dihydro-1H-inden-5-yl]oxy}methyl)pyridine-3-carbonitrile (0.7 g, 1.48 mmol) and 2-(azidomethyl)pyrrolidine hydrochloride (2, 0.36 g, 2.21 mmol) in N,N-dimethylformamide (10 mL) and methanol (10 mL) was added triethylamine (0.62 mL, 4.43 mmol) and acetic acid (0.42 mL, 7.38 mmol) under nitrogen atmosphere at room temperature and the reaction mixture was stirred for 6 h at 70° C. To this reaction mass, was added sodium cyanoborohydride (0278 g, 4.43 mmol) and stirred at the same temperature for further 16 h. After completion of the reaction as monitored by TLC, the reaction mixture was diluted with water (10 mL) and extracted with 10% methanol in dichloromethane (3×15 mL). The organic layer was dried over anhydrous sodium sulfate and concentrated. The resulting crude was purified by silica gel flash column chromatography using 10% methanol in dichloromethane as eluent to get the desired product (3, 0.45 g, 52.17%) as brown solid. LCMS (ES): m/z=585.5 [M+H]⁺.

### Step-3: Synthesis of 5-(((4-((2-(aminomethyl)pyrrolidin-1-yl)methyl)-7-((2-methyl-[1,1′-biphenyl]-3-yl)methoxy)-2,3-dihydro-1H-inden-5-yl)oxy)methyl)nicotinonitrile (Example 53)

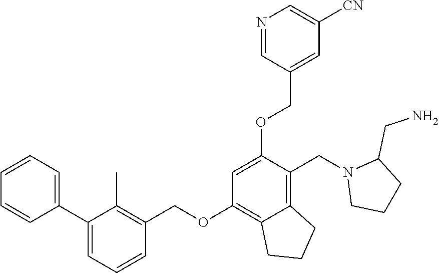 [SMILES: Cc1c(COc2cc(OCc3cncc(C#N)c3)c(CN3CCCC3CN)c3c2CCC3)cccc1-c1ccccc1]

To a stirred solution of 5-{[(4-{[2-(azidomethyl)pyrrolidin-1-yl]methyl}-7-({2-methyl-[1,1′-biphenyl]-3-yl}methoxy)-2,3-dihydro-1H-inden-5-yl)oxy]methyl}pyridine-3-carbonitrile (3, 0.450 g, 0.77 mmol) in tetrahydrofuran (10 mL) and water (0.5 mL) was added triphenylphosphine (0.428 g, 1.54 mmol) under nitrogen atmosphere at room temperature and the reaction mixture was stirred for 12 h at room temperature. After completion of the reaction as monitored by TLC, the reaction mixture was diluted with water (15 mL) and extracted with ethyl acetate (2×20 mL). The combined organic layer was washed with brine solution, dried over sodium sulphate, concentrated under reduced pressure and the crude was purified by silica gel column chromatography to obtain the title compound (Example 53, 0.25 g, 58.14%) as white solid. LCMS (ES) m/z=559.35 [M+H]⁺. ¹H NMR (400 MHz, DMSO-d₆) δ ppm: 8.98-8.97 (m, 2H), 8.41 (d, J=2 Hz, 1H), 8.36 (s, 1H), 7.47-7.36 (m, 4H), 7.33-7.31 (m, 2H), 7.26 (t, J=7.2 Hz, 1H), 7.19 (d, J=6.8 Hz, 1H), 6.74 (s, 1H), 5.33-5.25 (m, 2H), 5.13 (s, 2H), 3.75 (d, J=12 Hz, 1H), 3.35 (d, J=12 Hz, 2H), 2.94-2.86 (m, 3H), 2.76-2.59 (m, 6H), 2.25-1.98 (m, 4H), 2.01-1.92 (m, 2H), 1.89-1.80 (m, 1H), 1.63-1.50 (m, 3H). HPLC: 97.71%.

### Example 54: Synthesis of 5-(((7-((3-bromo-2-methylbenzyl)oxy)-4-((2-(hydroxymethyl)azetidin-1-yl)methyl)-2,3-dihydro-1H-inden-5-yl)oxy)methyl)nicotinonitrile and

### Example 55: 5-(((7-((4′-hydroxy-2-methyl-[1,1′-biphenyl]-3-yl)methoxy)-4-((2-(hydroxymethyl)azetidin-1-yl)methyl)-2,3-dihydro-1H-inden-5-yl)oxy)methyl)nicotinonitrile

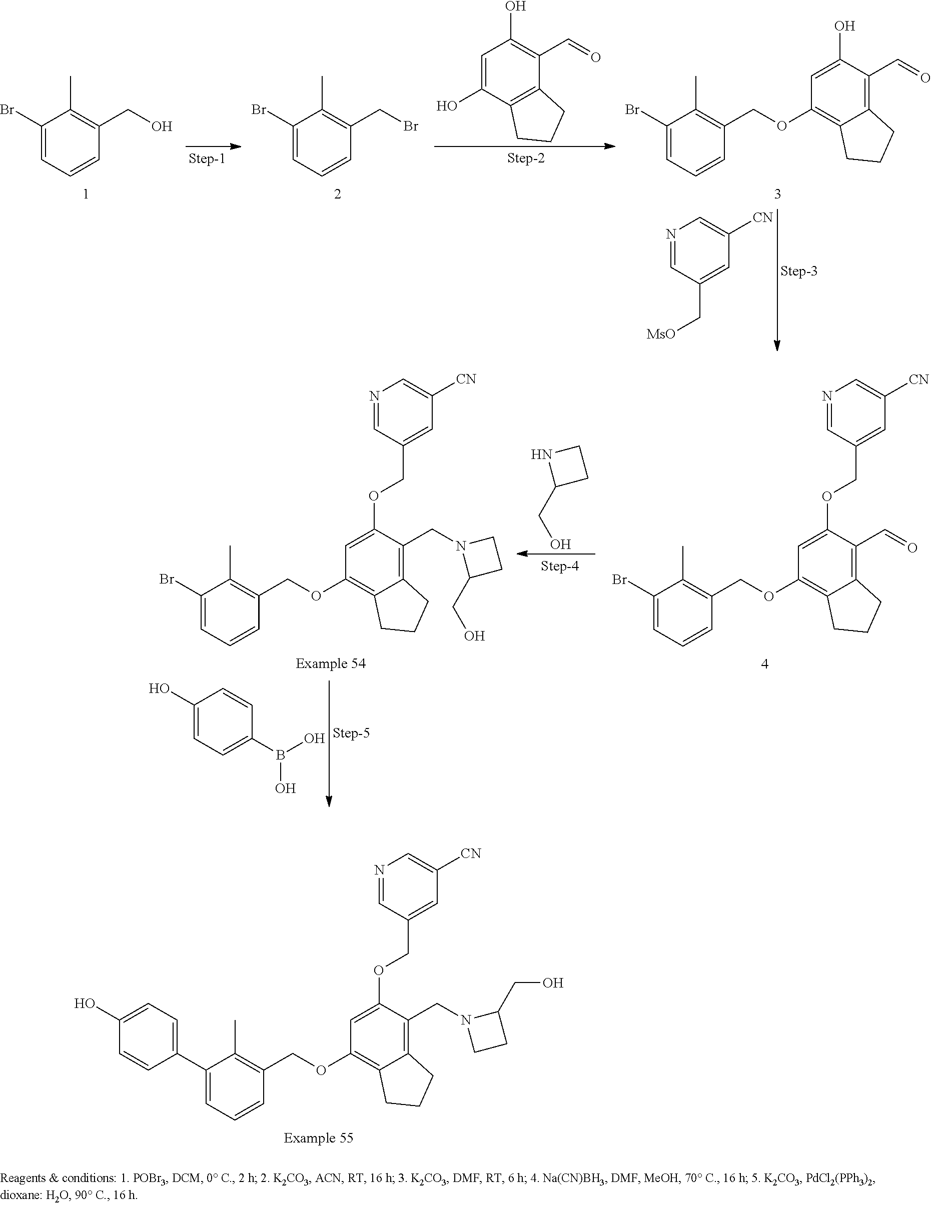

### Step-1: Synthesis of 1-bromo-3-(bromomethyl)-2-methylbenzene (2)

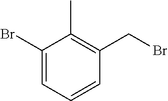

To a stirred solution of (3-bromo-2-methylphenyl)methanol (1, 10 g, 49.7 mmol) in dichloromethane (60 mL) was added tribromophosphane (21.4 g, 74.8 mmol) at 0° C. under nitrogen atmosphere. The resulting solution was stirred for further 2 h. The reaction was quenched with aqueous sodium bicarbonate (200 mL) solution. The organic layer was separated, dried over sodium sulphate, and concentrated under reduced pressure to obtain the desired product (2, 7.91 g, 61% yield) as an off white solid. ¹H NMR (400 MHz, DMSO-d₆) δ ppm: 7.58 (d, J=8 Hz, 1H), 7.44 (t, J=7.6 Hz, 3H), 7.12 (t, J=8 Hz, 1H), 4.88 (s, 2H), 2.41 (s, 3H).

### Step-2: Synthesis of 7-((3-bromo-2-methylbenzyl)oxy)-5-hydroxy-2,3-dihydro-1H-indene-4-carbaldehyde (3)

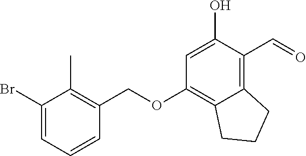 [SMILES: Cc1c(Br)cccc1COc1cc(O)c(C=O)c2c1CCC2]

To a stirred solution of 5,7-dihydroxy-2,3-dihydro-1H-indene-4-carbaldehyde (4.2 g, 23.6 mmol) in acetonitrile (150 mL) was added potassium carbonate (6.51 g, 47.2 mmol) and 1-bromo-3-(bromomethyl)-2-methylbenzene (2, 6.18 g, 23.6 mmol) at room temperature and stirred the reaction mixture for 16 h at room temperature. After completion of the reaction as monitored by TLC, the reaction mixture was diluted with water (30 mL) and solid suspension was filtered and dried in vacuo to get the desired product (3, 6.0 g, 71%) as brownish solid. LCMS (ES) m/z=361.26 [M+H]⁺.

### Step-3: Synthesis of 5-(((7-((3-bromo-2-methylbenzyl)oxy)-4-formyl-2,3-dihydro-1H-inden-5-yl)oxy)methyl)nicotinonitrile (4)

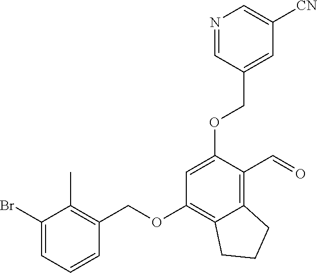 [SMILES: Cc1c(Br)cccc1COc1cc(OCc2cncc(C#N)c2)c(C=O)c2c1CCC2]

To a stirred solution of 7-((3-bromo-2-methylbenzyl)oxy)-5-hydroxy-2,3-dihydro-1H-indene-4-carbaldehyde (3, 3.8 g, 10.0 mmol) in N,N-dimethylformamide (60 mL) was added potassium carbonate (2.76 g, 20 mmol) and (5-cyanopyridin-3-yl)methyl methanesulfonate (2.68 g, 12.02 mmol) at room temperature. The reaction mass was stirred for further 6 h at the same temperature. After completion, the reaction mixture was diluted with water (20 mL) and solid suspension was appeared. This solid was filtered and dried in vacuo to obtain the desired product (4, 4.5 g, 90%) as a grey solid. LCMS (ES) m/z=477 [M+H]⁺.

### Step-4: Synthesis of 5-(((7-((3-bromo-2-methylbenzyl)oxy)-4-((2-(hydroxymethyl)azetidin-1-yl)methyl)-2,3-dihydro-1H-inden-5-yl)oxy)methyl)nicotinonitrile (Example 54)

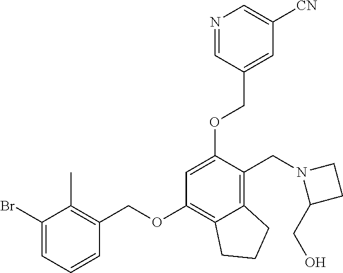 [SMILES: Cc1c(Br)cccc1COc1cc(OCc2cncc(C#N)c2)c(CN2CCC2CO)c2c1CCC2]

To a stirred solution of 5-({[7-({4′-fluoro-2-methyl-[1,1′-biphenyl]-3-yl}methoxy)-4-formyl-2,3-dihydro-1H-inden-5-yl]oxy}methyl)pyridine-3-carbonitrile (4, 3 g, 5.48 mmol) and (azetidin-2-yl)methanol (1.9 g, 13.7 mmol) in N,N-dimethylformamide (45 mL) and methanol (36 mL), acetic acid (0.2 mL) was added under nitrogen atmosphere at room temperature and the reaction mixture was stirred for 1 h at 70° C. Sodium cyanoborohydride (1.03 g 16.4 mmol) was added portion wise to the reaction mixture and stirred at the same temperature for further 6 h. After completion of the reaction as monitored by TLC, the reaction mixture was diluted with water (10 mL) and extracted with ethyl acetate (3×150 mL). The organic layer was dried over anhydrous sodium sulfate and concentrated. The resulting crude was purified by neutral alumina column chromatography using 10% methanol in dichloromethane as eluent to afford the title compound (Example 54, 1.7 g, 56%) as a light brown semi solid. LCMS (ES) m/z=548.5 [M+H]⁺. ¹H NMR (400 MHz, DMSO-d₆) δ ppm: 9.00-8.98 (m, 2H), 8.49 (s, 1H), 7.58 (d, J=8 Hz, 1H), 7.43 (d, J=7.2 Hz, 1H), 7.14 (t, J=7.6 Hz, 1H), 6.70 (s, 1H), 5.28-5.13 (m, 2H), 5.13 (s, 2H), 4.23 (bs, 1H), 3.57 (d, J=9.6 Hz, 1H), 3.46 (d, J=12.0 Hz, 1H), 3.23-3.13 (m, 3H), 3.07-3.00 (m, 1H), 2.95-2.85 (m, 2H), 2.82-2.70 (m, 3H), 2.38 (s, 3H), 2.00-1.91 (m, 2H), 1.90-1.80 (m, 1H), 1.79-1.70 (m, 1H). HPLC: 95.33%.

### Step-5: Synthesis of 5-(((7-((4′-hydroxy-2-methyl-[1,1′-biphenyl]-3-yl)methoxy)-4-((2-(hydroxymethyl)azetidin-1-yl)methyl)-2,3-dihydro-1H-inden-5-yl)oxy)methyl)nicotinonitrile (Example 55)

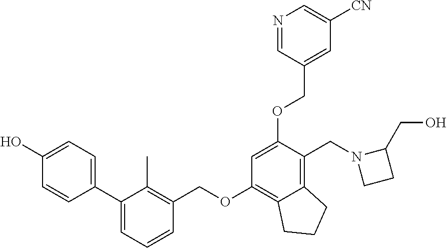 [SMILES: Cc1c(COc2cc(OCc3cncc(C#N)c3)c(CN3CCC3CO)c3c2CCC3)cccc1-c1ccc(O)cc1]

To a stirred solution of 5-(((7-((3-bromo-2-methylbenzyl)oxy)-4-((2-(hydroxymethyl)azetidin-1-yl)methyl)-2,3-dihydro-1H-inden-5-yl)oxy)methyl)nicotinonitrile (Example 54, 0.2 g, 0.36 mmol) in 1,4-dioxane:water (6:1, 12 mL), 4-hydroxy benzene boronic acid (0.060 g, 0.43 mmol) was added and the reaction mixture was purged with argon for 10 min. potassium carbonate (0.151 g, 1.09 mmol) and PdCl₂(PPh₃)₂ (0.025 g, 0.36 mmol) were sequentially added. The reaction mixture was sealed and stirred for 16 h at 90° C. After completion, the reaction mixture was poured into water (30 mL) and the aqueous layer was extracted with ethyl acetate (3×40 mL). The organic layers were combined, dried (Na₂SO₄) and concentrated in vacuo to give crude. The residue was purified by flash column chromatography [neutral Al₂O₃, gradient 2% to 3% methanol in dichloromethane] to give the title compound (Example 55, 0.021 g, 9%) as a grey solid. LCMS (ES) m/z=562.37 [M+H]⁺; ¹H NMR (400 MHz, DMSO-d₆) δ ppm: 9.00 (s, 2H), 9.49 (bs, 1H), 8.51 (s, 1H), 7.38 (t, J=7.2 Hz, 1H), 7.22 (t, J=7.6 Hz, 1H), 7.15 (s, 1H), 7.10-7.14 (m, 2H), 6.81 (d, J=8.4 Hz, 2H), 6.73 (s, 1H), 5.27 (dd, J=12.8, 8.4 Hz, 2H), 5.12 (s, 2H), 4.24 (bs, 1H), 3.62-3.52 (m, 1H), 3.50-3.40 (m 1H), 3.23-3.13 (m, 3H), 3.05-3.00 (m, 1H), 2.92-2.83 (m, 2H), 2.80-2.78 (m, 1H), 2.74 (t, J=7.2 Hz, 2H), 2.20 (s, 3H), 2.00-1.92 (m, 2H), 1.89-1.80 (m, 1H), 1.77-1.70 (m, 1H). HPLC: 96.63%.

The compounds listed in below Table-7 were prepared by a procedure similar to the one described in Example-55 with appropriate variations in reactants, quantity of reagents, protections & deprotections, solvents & reaction conditions. The characterization data of the compounds are summarized herein below table.

TABLE 7

| Example | | |
|---|---|---|
| No. | Structure | Characterization data |
| 56 | 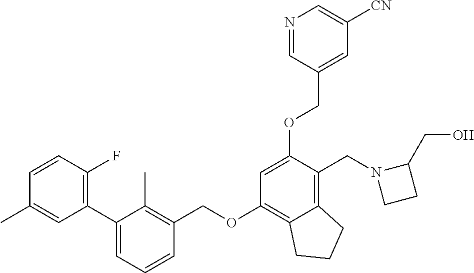 [SMILES: Cc1ccc(F)c(-c2cccc(COc3cc(OCc4cncc(C#N)c4)c(CN4CCC4CO)c4c3CCC4)c2C)c1] | LCMS (ES) m/z = 578.31 [M + H]⁺; ¹H NMR (400 MHz, DMSO-d₆) δ ppm: 9.00 (s, 2H), 8.51 (s, 1H), 7.48 (d, J = 6.8 Hz, 1H), 7.31-7.24 (m, 2H), 7.19 (d, J = 9.6 Hz, 2H), 7.12 (d, J = 7.2 Hz, 1H), 6.75 (s, 1H), 5.30-5.20 (m, 2H), 5.15 (s, 2H), 4.25 (bs, 1H), 3.62-3.55 (m, 1H), 3.50- 3.40 (m, 1H), 3.28-3.15 (m, 3H), 3.06-3.00 (m, 1H), 2.96-2.86 (m, 3H), 2.73 (t, J = 7.2 Hz, 2H), 2.33 (s, 3H), 2.13 (s, 3H), 1.91-2.02 (m, 2H), 1.90-1.80 (m, 1H), 1.79-1.70 (m, 1H); HPLC: 95.51%. |
| 57 | 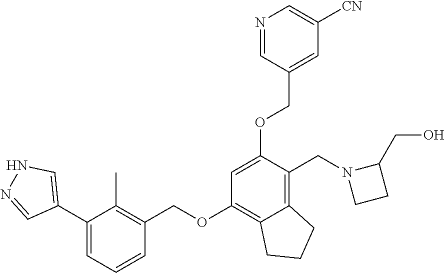 [SMILES: Cc1c(COc2cc(OCc3cncc(C#N)c3)c(CN3CCC3CO)c3c2CCC3)cccc1-c1cn[nH]c1] | LCMS (ES) m/z = 536.31 [M + H]⁺; ¹H NMR (400 MHz, DMSO-d₆) δ ppm: 13.00 (bs, 1H), 9.00 (s, 2H), 8.51 (t, J = 2.0 Hz, 1H), 7.93 (s, 1H), 7.66 (s, 1H), 7.32 (t, J = 8.0 Hz, 2H), 7.19 (t, J = 7.6 Hz, 1H), 6.74 (s, 1H), 5.30-5.21 (m, 2H), 5.11 (s, 2H), 4.24 (bs, 1H), 3.57 (d, J = 11. 6 Hz, 1H), 3.46 (d, J = 12 Hz, 1H), 3.23 - 3.15 (m, 3H), 3.04-3.01 (m, 1H), 2.94- 2.76 (m, 3H), 2.72 (t, J = 6.0 Hz, 2H), 2.33 (s, 3H), 1.92-2.00 (m, 2H), 1.90-1.80 (m, 1H), 1.68-1.77 (m, 1H); HPLC: 98.68%. |
| 58 | 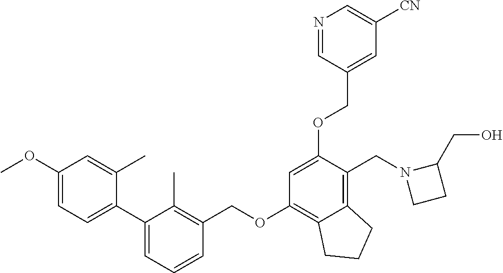 | LCMS (ES) m/z = 590.35 [M + H]⁺; ¹H NMR (400 MHz, DMSO-d₆) δ ppm: 9.00 (s, 2H), 8.52 (s, 1H), 7.41 (d, J = 6.8 Hz, 1H), 7.23 (t, J = 7.6 Hz, 1H), 7.04 (d, J = 6.8 Hz, 1H), 6.98 (d, J = 6.98 Hz, 1H) , 6.88 (d, J = 2.8 Hz, 1H), 6.81 (dd, J = 8.8, 2.8 Hz, 1H), 6.73 (s, 1H), 5.30-5.20 (m, 2H), 5.12 (s, 2H), 4.24 (bs, 1H), 3.77 (s, 3H), 3.47 (t, J = 12.8 Hz, 1H), 3.58 (d, J = 11.6 Hz, 1H), 3.23-3.12 (m, 3H), 3.04-3.00 (m, 1H), 2.98- 2.78 (m, 3H), 2.73 (t, J = 7.2 Hz, 2H), 2.00 (s, 3H), 1.95 (s, 3H), 2.00- 1.91 (m, 2H), 1.90-1.80 (m, 1H), 1.79-1.70 (m, 1H); HPLC: 91.53%. |
| 59 | 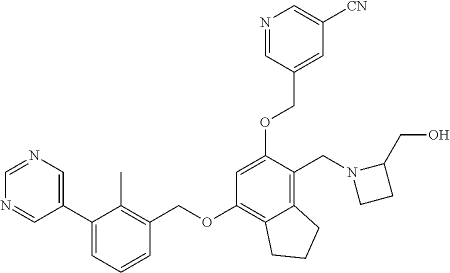 | LCMS (ES) m/z = 548.38 [M + H]⁺; ¹H NMR (400 MHz, DMSO-d₆) δ ppm: 9.23 (s, 1H), 9.00 (s, 2H), 8.86 (s, 2H), 8.51 (t, J = 2.4 Hz, 1H), 7.54 (d, J = 6.4 Hz, 1H), 7.40-7.30 (m, 2H), 6.74 (s, 1H), 5.26 (dd, J = 12.4, 8.4 Hz, 2H), 5.16 (s, 2H), 4.26 (bs, 1H), 3.57-3.52 (m, 1H), 3.50-3.46 (m, 1H), 3.25-3.17 (m, 3H), 3.05- 3.00 (m, 1H), 2.91-2.82 (m, 3H), 2.75 (t, J = 7.2 Hz, 2H), 2.24 (s, 3H), 2.00-1.91 (m, 2H), 1.90-1.80 (s, 1H), 1.78-1.70 (m, 1H); HPLC: 90.18%. |
| 60 | 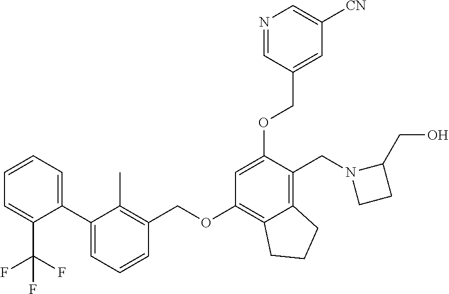 | LCMS (ES) m/z = 614.35 [M + H]⁺; 1H NMR (400 MHz, DMSO-d₆) δ ppm: 9.00 (d, J = 1.6 Hz, 2H), 8.51 (s, 1H), 7.84 (d, J = 7.6 Hz, 1H), 7.73 (t, J = 7.2 Hz, 1H), 7.63 (t, J = 7.6 Hz, 1H), 7.48 (d, J = 7.2 Hz, 1H), 7.33 (d, J = 7.6 Hz, 1H), 7.25 (t, J = 8 Hz, 1H), 7.10 (d, J = 7.2 Hz, 1H), 6.73 (s, 1H), 5.24 (dd, J = 12.4, 8.4 Hz, 2H), 5.13 (s, 2H), 4.24 (bs, 1H), 3.62-3.53 (m, 1H), 3.50- 3.40 (m, 1H), 3.23-3.13 (m, 2H), 3.05-3.00 (m, 2H), 2.92-2.83 (m, 3H), 2.72 (t, J = 7.2 Hz, 2H), 1.96 (s, 3H), 2.00-1.90 (m, 2H), 1.89-1.80 (m, 1H), 1.77-1.70 (m, 1H); HPLC: 93.88%. |
| 61 | 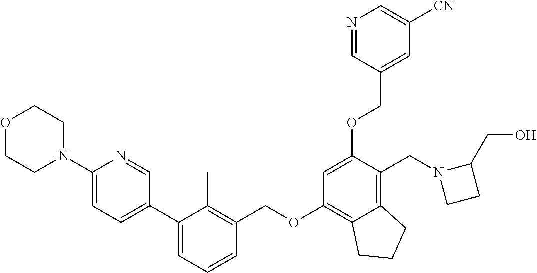 | LCMS (ES) m/z = 632.38 [M + H]⁺; ¹H NMR (400 MHz, DMSO-d₆) δ ppm: 9.00 (s, 2H), 8.51 (s, 1H), 8.10 (d, J = 2.4 Hz, 1H), ), 7.58-7.55 (m, 1H), 7.42 (d, J = 6.8 Hz, 1H), 7.25 (t, J = 7.6 Hz, 1H), 7.19 (d, J = 7.6 Hz, 1H), 6.91 (d, J = 8.8 Hz, 1H), 6.74 (s, 1H), 5.26 (q, J = 10.5 Hz, 2H), 5.13 (s, 2H), 4.26 (bs, 1H), 3.72 (t, J = 4.4 Hz, 4H), 3.59 (s, 1H), 3.47 (t, J = 4.8 Hz, 5H), 3.21 (s, 3H), 3.03 (s, 1H), 2.94-2.79 (m, 3H), 2.74 ( t, J = 7.2 Hz, 2H), 2.23 (s, 3H), 2.00- 1.93 (m, 2H), 1.88-1.81 (m, 1H), 1.71-1.77 (m, 1H); HPLC: 92.34%. |
| 62 | 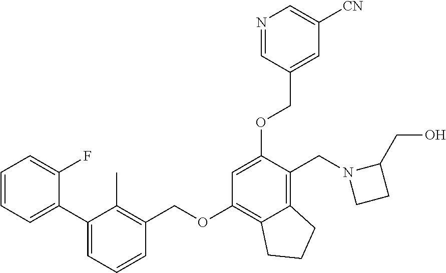 | LCMS (ES) m/z = 564.32 [M + H]⁺; ¹H NMR (400 MHz, DMSO-d₆) δ ppm: 9.01 (s, 2H), 8.52 (s, 1H), 7.50- 7.44 (m, 2H), 7.35-7.27 (m, 4H), 7.20 (d, J = 7.6 Hz, 1H), 6.77 (s, 1H), 5.31-5.23 (m, 2H), 5.10 (s, 2H), 4.26 (bs, 1H), 3.63-3.40 (m, 2H), 3.35- 3.00 (m, 3H), 2.98-2.70 (m, 6H), 2.13 (s, 3H), 1.99-1.92 (m, 3H), 1.75 (bs, 1H); HPLC: 93.73%. |
| 63 | 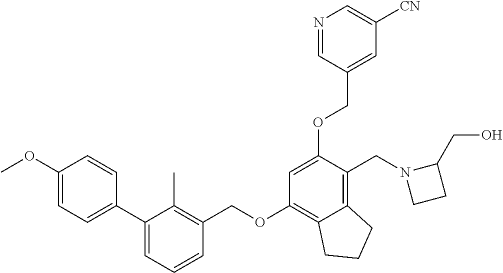 | LCMS (ES) m/z = 576.33 [M + H]⁺; ¹H NMR (400 MHz, DMSO-d₆) δ ppm: 9.00 (s, 2H), 8.52 (s, 1H), 7.41 (d, J = 6.8 Hz 1H), 7.26-7.22 (m, 3H), 7.17 (d, J = 6.8 Hz, 1H), 7.01 (d, J = 8.8 Hz, 2H), 6.73 (s, 1H), 5.24 (q, J = 11.6 Hz, 2H), 5.12 (s, 2H), 4.26 (bs, 1H), 3.79 (s, 3H), 3.58 (d, J = 12 Hz, 1H), 3.45 (d, J = 12 Hz, 1H), 3.18-3.21 (m, 3H), 3.02 (t, J = 6 Hz, 1H), 2.92-2.85 (m, 3H), 2.73 (t, J = 7.20 Hz, 2H), 2.21 (s, 3H), 2.00-1.92 (m, 2H), 1.88-1.82 (m, 1H), 1.79-1.70 (m, 1H); HPLC: 97.26%. |
| 64 | 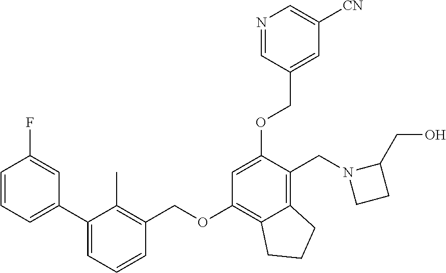 [SMILES: Cc1c(COc2cc(OCc3cncc(C#N)c3)c(CN3CCC3CO)c3c2CCC3)cccc1-c1cccc(F)c1] | LCMS (ES) m/z = 564.35 [M + H]⁺; ¹H NMR (400 MHz, DMSO-d₆) δ ppm: 9.00 (d, J = 1.6 Hz, 2H), 8.51 (s, 1H), 7.55-7.46 (m, 2H), 7.16-7.29 (m, 5H), 6.73 (s, 1H), 5.22-5.30 (m, 2H), 5.14 (s, 2H), 4.26 (bs, 1H), 3.57 (d, J = 12 Hz, 1H), 3.45 (d, J = 12 Hz, 1H), 3.22-3.15 (m, 3H), 3.06- 3.01 (m, 1H), 2.93-2.84 (m, 3H), 2.74 (t, J = 7.2 Hz, 2H), 2.21 (s, 3H), 1.98-1.95 (m, 2H), 1.90-1.80 (m, 1H), 1.77-1.68 (m, 1H); HPLC: 92.56%. |
| 65 | 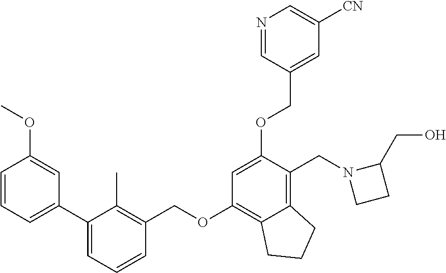 [SMILES: COc1cccc(-c2cccc(COc3cc(OCc4cncc(C#N)c4)c(CN4CCC4CO)c4c3CCC4)c2C)c1] | LCMS (ES) m/z = 576.36 [M + H]⁺; ¹H NMR (400 MHz, DMSO-d₆) δ ppm: 9.00 (d, J = 1.6 Hz, 2H), 8.52 (s, 1H), 7.44 (d, J = 7.2 Hz, 1H), 7.38 (t, J = 7.6 Hz, 1H), 7.18-7.27 (m, 2H), 6.94 (dd, J = 8.4, 2.4 Hz, 1H), 6.86 (d, J = 8.4 Hz, 1H), 6.84 (s, 1H), 6.74 (s, 1H), 5.31-5.20 (m, 1H), 5.13 (s, 2H), 3.78 (s, 3H), 3.60- 3.55 (m, 1H), 3.43-3.48 (m, 1H), 3.15-3.25 (m, 3H), 3.05-3.00 (m, 1H). 2.79-2.93 (m, 3H), 2.74 (t, J = 7.2 Hz, 2H), 2.72-2.66 (m, 2H), 2.21 (s, 3H), 1.98-1.90 (m, 2H), 1.90-1.80 (m, 1H), 1.77-1.68 (m, 1H); HPLC: 93.36%. |
| 66 | 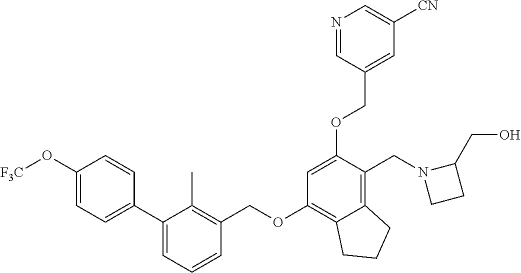 [SMILES: Cc1c(COc2cc(OCc3cncc(C#N)c3)c(CN3CCC3CO)c3c2CCC3)cccc1-c1ccc(OC(F)(F)F)cc1] | LCMS (ES) m/z = 630.33 [M + H]⁺; ¹H NMR (400 MHz, DMSO-d₆) δ ppm: 9.00 (s, 2H), 8.51 (s, 1H), 7.43- 7.40 (m, 5H), 7.28 (t, J = 7.6 Hz, 1H), 7.22 (d, J = 6.8 Hz, 1H), 6.74 (s, 1H), 5.26 (q, J = 9.6 Hz, 2H), 5.14 (s, 2H), 4.26 (bs, 1H), 3.57 (d, J = 12 Hz, 1H), 3.47 (t, J = 10.8, 1H), 3.17-3.25 (m, 3H), 3.15-3.00 (m, 1H), 2.95-2.82 (m, 3H), 2.72 (t, J = 7.2 Hz, 2H), 2.20 (s, 3H), 2.00-1.92 (m, 2H), 1.90-1.80 (m, 1H), 1.77- 1.68 (m, 1H); HPLC: 93.76%. |
| 67 | 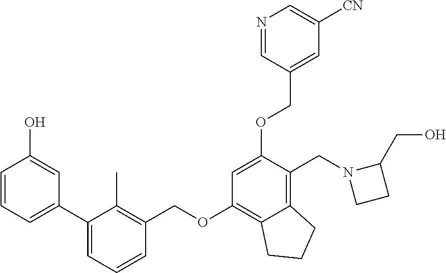 [SMILES: Cc1c(COc2cc(OCc3cncc(C#N)c3)c(CN3CCC3CO)c3c2CCC3)cccc1-c1cccc(O)c1] | LCMS (ES) m/z = 562.35 [M + H]⁺; ¹H NMR (400 MHz, DMSO-d₆) δ ppm: 9.48 (bs, 1H), 9.00 (s, 2H), 8.52 (s, 1H), 7.41 (d, J = 6.8 Hz 1H), 7.28-7.20 (m, 2H), 7.15 (d, J = 6.8 Hz, 1H), 6.73-6.23 (m, 2H), 6.73 (s, 1H), 6.72-6.68 (m, 1H), 5.24 (q, J = 11.6 Hz, 2H), 5.13 (s, 2H), ), 4.22 (bs, 1H), 3.58 (d, J = 12 Hz, 1H), 3.45 (d, J = 12 Hz, 1H), 3.21-3.18 (m, 3H), 3.05-3.00 (m, 1H), 2.98- 2.80 (m, 3H), 2.72 (t, J = 7.20 Hz, 2H), 2.20 (s, 3H), 2.00-1.92 (m, 2H), 1.88-1.82 (m, 1H), 1.79-1.70 (m, 1H); HPLC: 98.84%. |
| 68 | 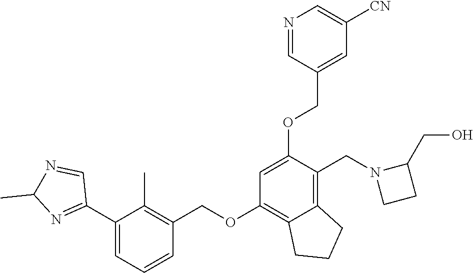 [SMILES: Cc1c(COc2cc(OCc3cncc(C#N)c3)c(CN3CCC3CO)c3c2CCC3)cccc1C1=NC(C)N=C1] | LCMS (ES) m/z = 550.4 [M + H]⁺; ¹H NMR (400 MHz, DMSO-d₆) δ ppm: 9.28 (bs, 1H), 9.02 (d, J = 1.6 Hz, 2H), 8.51 (s, 1H), 7.90 (s, 1H), 7.60 (s, 1H), 7.38-7.29 (m, 2H), 7.19 (t, J = 7.6 Hz, 1H), 6.85 (s, 1H), 5.41- 5.30 (m, 3H), 5.19 (s, 2H), 4.45 (bs, 1H), 4.35-4.20 (m, 2H), 3.95-3.79 (m, 5H), 3.57 (bs, 2H), 3.00-2.88 (m, 2H), 2.76 (t, J = 7.20 Hz, 2H), 2.33 (s, 3H), 2.22-2.10 (m, 2H), 2.07-1.95 (m, 2H); HPLC: 92.01%. |
| 69 | 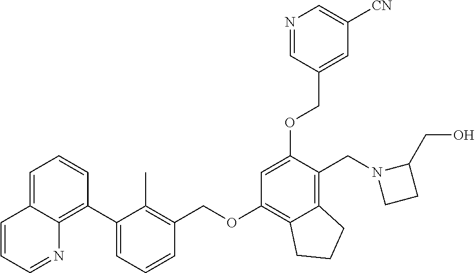 [SMILES: Cc1c(COc2cc(OCc3cncc(C#N)c3)c(CN3CCC3CO)c3c2CCC3)cccc1-c1cccc2cccnc12] | LCMS (ES) m/z = 597.46 [M + H]⁺; ¹H NMR (400 MHz, DMSO-d₆) δ ppm: 9.00 (t, J = 2.4 Hz, 2H), 8.82- 8.80 (m, 1H), 8.52 (t, J = 2.0 Hz, 1H), 8.43 (dd, J = 8.0, 1.6 Hz, 1H), 8.03 (dd, J = 8.0, 1.2 Hz, 1H), 7.69 (t, J = 6.8 Hz, 1H), 7.63-7.61 (m, 1H), 7.56-7.53 (m, 1H), 7.48 (d, J = 6.8 Hz, 1H), 7.28 (t, J = 7.6 Hz, 1H), 7.21-7.19 (m, 1H), 6.77 (s, 1H), 5.31-5.23 (m, 2H), 5.13 (s, 2H), 4.23 (bs, 1H), 3.57 (d, J = 10.8 Hz, 1H), 3.48 (d, J = 11.6 Hz, 1H), 3.23-3.12 (m, 3H), 3.09-3.00 (m, 1H), 2.98- 2.83 (m, 2H), 2.82-2.71 (m, 3H), 2.00-1.92 (m, 5H), 1.84-1.85 (m, 1H), 1.77-1.73 (m, 1H); HPLC: 97.36%. |
| 70 | 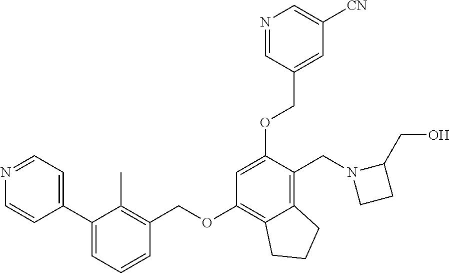 [SMILES: Cc1c(COc2cc(OCc3cncc(C#N)c3)c(CN3CCC3CO)c3c2CCC3)cccc1-c1ccncc1] | LCMS (ES) m/z = 547.34 [M + H]⁺; ¹H NMR (400 MHz, DMSO-d₆) δ ppm: 9.00 (s, 2H), 8.64 (d, J = 6.0 Hz , 2H), 8.51 (s, 1H), 7.52 (d, J = 7.6 Hz, 1H), 7.39 (d, J = 6.0 Hz, 2H), 7.32 (t, J = 7.6 Hz, 1H), 7.23 (d, J = 7.2 Hz, 1H), 6.74 (s, 1H), 5.30-5.20 (m, 2H), 4.26 (bs, 1H), 5.15 (s, 2H), 3.55-3.62 (m, 1H), 3.40-3.50 (m, 1H), 3.33-3.38 (m, 1H), 3.12-3.25 (m, 3H), 3.00-3.06 (m, 1H), 2.85-2.96 (m, 2H), 2.74 (t, J = 7.2 Hz, 2H), 2.22 (s, 3H), 1.92- 2.02 (m, 2H), 1.90-1.80 (m, 1H), 1.79-1.70 (m, 1H); HPLC: 96.15%. |
| 71 | 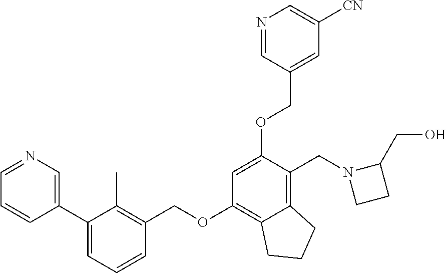 [SMILES: Cc1c(COc2cc(OCc3cncc(C#N)c3)c(CN3CCC3CO)c3c2CCC3)cccc1-c1cccnc1] | LCMS (ES) m/z = 547.4 [M + H]⁺; ¹H NMR (400 MHz, DMSO-d₆) δ ppm: 9.00 (s, 2H), 8.60 (d, J = 6.0 Hz , 1H), 8.55 (s, 1H), 8.51 (s, 1H), 7.81- 7.77 (m, 1H), 7.52-7.45 (m, 2H), 7.31 (t, J = 7.6 Hz, 1H), 7.24 (d, J = 6.8 Hz, 1H), 6.71 (s, 1H), 5.20-5.30 (m, 2H), 5.15 (s, 2H), 4.23 (bs, 1H), 3.60-3.55 (m, 1H), 3.45-3.50 (m, 1H), 3.38-3.33 (m, 1H), 3.25-3.12 (m, 3H), 3.06-3.00 (m, 1H), 2.85- 2.96 (m, 2H), 2.74 (t, J = 7.2 Hz, 2H), 2.22 (s, 3H), 2.02-1.91 (m, 2H), 1.90-1.80 (m, 1H), 1.79-1.70 (m, 1H); HPLC: 90.69%. |
| 72 | 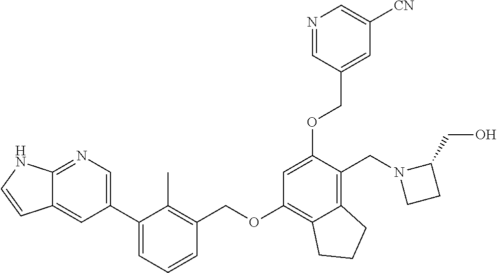 [SMILES: Cc1c(COc2cc(OCc3cncc(C#N)c3)c(CN3CC[C@H]3CO)c3c2CCC3)cccc1-c1cnc2[nH]ccc2c1] | LCMS (ES) m/z = 586.3 [M + H]⁺; ¹H NMR (400 MHz, DMSO-d₆) δ ppm: 11.72 (bs, 1H), 9.00 (s, 2H), 8.51 (s, 1H), 8.15 (s, 1H), 7.89 (s, 1H), 7.52 (t, J = 2.8 Hz, 1H), 7.46 (dd, J = 7.2, 2.4 Hz, 1H), 7.32-7.23 (m, 2H), 6.75 (s, 1H), 6.48 (s, 1H), 5.27 (q, J = 12.8 Hz, 2H), 5.16 (s, 2H), 4.23 (s, 1H), 3.56 (t, J = 12.4 Hz, 1H), 3.46 (d, J = 12.0 Hz, 1H), 3.20-3.10 (m, 3H), 3.09-3.00 (m, 1H), 2.90-2.70 (m, 5H), 2.23 (s, 3H), 2.00-1.91 (m, 2H), 1.90-1.70 (m, 2H); HPLC: 95.54%. |
| 73 | 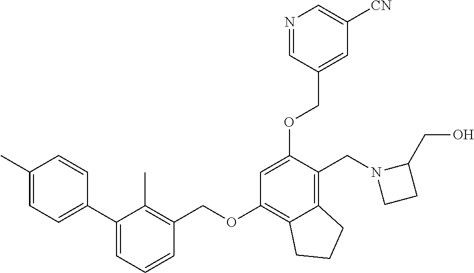 [SMILES: Cc1ccc(-c2cccc(COc3cc(OCc4cncc(C#N)c4)c(CN4CCC4CO)c4c3CCC4)c2C)cc1] | LCMS (ES) m/z = 560.45 [M + H]⁺; ¹H NMR (400 MHz, DMSO-d₆) δ ppm: 9.00 (s, 2H), 8.51 (s, 1H), 7.42 (d, J = 7.2 Hz, 1H), 7.30-7.16 (m, 6H), 6.73 (s, 1H), 5.26 (q, J = 10.2 Hz, 2H), 5.13 (s, 2H), 4.24 (bs, 1H), 3.58 (d, J = 11.6 Hz, 1H), 3.47 (t, J = 12.8 Hz, 1H), 3.25-3.18 (m, 3H), 3.06-3.00 (m, 1H), 2.97-2.78 (m, 3H),2.73 (t, J = 7.2 Hz, 2H), 2.36 (s, 3H), 2.20 (s, 3H), 2.04-1.94 (m, 2H), 1.78-1.70 (m, 1H), 1.90-1.80 (m, 1H); HPLC: 98.11%. |
| 74 | 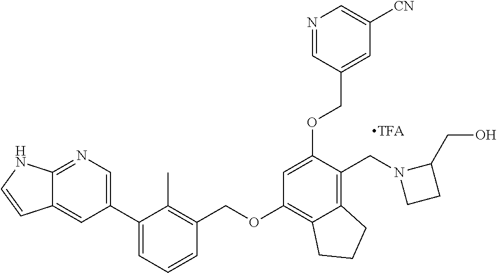 [SMILES: Cc1c(COc2cc(OCc3cncc(C#N)c3)c(CN3CCC3CO)c3c2CCC3)cccc1-c1cnc2[nH]ccc2c1] | LCMS (ES) m/z = 586.30 [M + H]⁺; ¹H NMR (400 MHz, DMSO-d₆) δ ppm: 11.74 (s, 1H), 9.09 (bs, 1H), 9.03 (s, 2H), 8.51 (s, 1H), 8.15 (s, 1H), 7.90 (s, 1H), 7.53 (t, J = 2.8 Hz, 1H), 7.42-7.50 (m, 1H), 7.30-7.25 (m, 2H), 6.88 (s, 1H), 6.49 (d, J = 2 Hz, 1H), 5.34 (q, J = 12.8 Hz, 2H), 5.23 (s, 2H), 4.50 (bs, 1H), 4.40-4.31 (m, 1H), 4.30-4.20 (m, 1H), 3.90- 3.80 (m, 2H), 3.60-3.50 (m, 3H), 2.98-2.92 (m, 2H), 2.80 (t, J = 7.6 Hz, 2H), 2.21 (s, 3H), 2.20-2.10 (m, 2H), 2.09-2.00 (m, 2H). HPLC: 99.61%. |
| 75 | 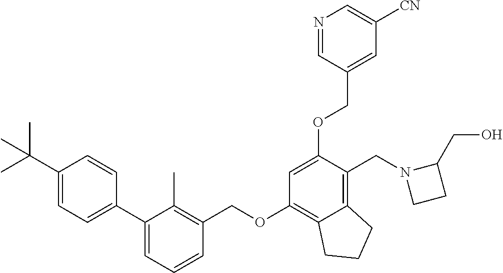 [SMILES: Cc1c(COc2cc(OCc3cncc(C#N)c3)c(CN3CCC3CO)c3c2CCC3)cccc1-c1ccc(C(C)(C)C)cc1] | LCMS (ES) m/z = 602.4 [M + H]⁺; ¹H NMR (400 MHz, DMSO-d₆) δ ppm: 9.00 (d, J = 1.6 Hz, 2H), 8.51 (s, 1H), 7.47 (d, J = 7.6 Hz, 2H), 7.42 (d, J = 6.8 Hz, 1H), 7.26-7.23 (m, 3H), 7.17 (d, J = 6.8 Hz, 1H), 6.74 (s, 1H), 5.30-5.20 (m, 2H), 5.13 (s, 2H), 4.24 (bs, 1H), 3.57 (d, J = 12 Hz, 1H), 3.45 (d, 12.4 Hz, 1H), 3.25- 3.12 (m, 3H), 3.06-3.00 (m, 1H), 2.96-2.80 (m, 3H), 2.74 (t, J = 7.2 Hz, 2H), 2.22 (s, 3H), 2.02-1.92 (m, 2H), 1.80-1.90 (m, 1H), 1.77-1.68 (m, 1H), 1.33 (s, 9H). HPLC: 98.07%. |
| 76 | 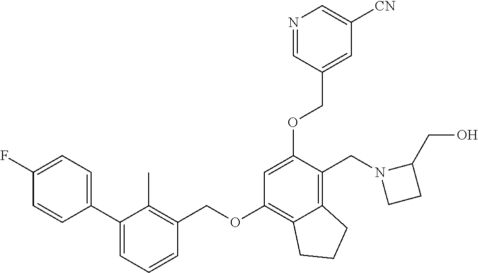 [SMILES: Cc1c(COc2cc(OCc3cncc(C#N)c3)c(CN3CCC3CO)c3c2CCC3)cccc1-c1ccc(F)cc1] | LCMS (ES) m/z = 564.4 [M + H]⁺; ¹H NMR (400 MHz, DMSO-d₆) δ ppm: 8.99 (d, J = 2.0 Hz, 2H), 8.51 (s, 1H), 7.45 (d, J = 7.6 Hz, 1H), 7.38- 7.34 (m, 2H), 7.31-7.22 (m, 3H), 7.19 (d, J = 6.8 Hz, 1H), 6.73 (s, 1H), 5.30-5.20 (m, 2H), 5.14 (s, 2H), 4.24 (t, J = 4.8 Hz, 1H), 3.58 (d, J = 12 Hz, 1H), 3.47 (t, J = 12 Hz, 1H), 3.35-3.12 (m, 3H), 3.08-2.70 (m, 6H), 2.19 (s, 3H), 2.00-1.98 (m, 2H), 1.90-1.80 (m, 1H), 1.79-1.70 (m, 1H). HPLC: 96.3%. |
| 77 | 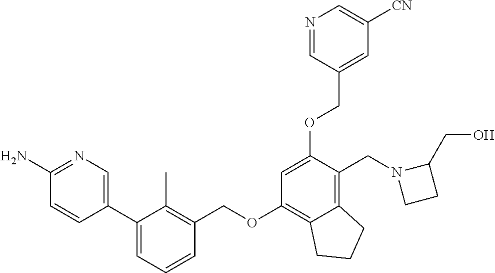 [SMILES: Cc1c(COc2cc(OCc3cncc(C#N)c3)c(CN3CCC3CO)c3c2CCC3)cccc1-c1ccc(N)nc1] | |
| 77A | 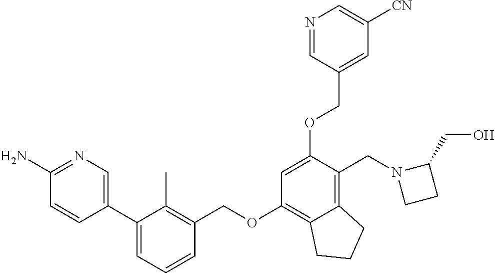 [SMILES: Cc1c(COc2cc(OCc3cncc(C#N)c3)c(CN3CC[C@H]3CO)c3c2CCC3)cccc1-c1ccc(N)nc1] | LCMS (ES) m/z = 562.35 [M + H]⁺; ¹H NMR (400 MHz, DMSO-d₆) δ ppm: 9.10 (bs, 1H), 9.03-9.01 (m, 2H), 8.49 (s, 1H), 7.94 (d, J = 4 Hz, 1H), 7.85 (d, J = 8 Hz, 1H), 7.70 (bs, 1H), 7.46 (d, J = 6.8 Hz, 1H), 7.37- 7.20 (m, 2H), 6.95 (d, J = 7.6 Hz, 1H), 6.85 (s, 1H), 6.48 (s, 1H), 5.39 (bs, 1H), 5.33 (s, 2H), 5.20 (s, 2H), 4.47 (bs, 1H), 4.38-4.23 (m, 2H), 3.98-3.75 (m, 2H), 3.58 (bs, 2H), 3.05 (bs, 1H), 3.00-2.90 (m, 2H), 2.77 (t, J = 7.6 Hz, 2H), 2.25-2.15 (m, 5H), 2.02 (bs, 1H). HPLC: 91%. |
| 81 | 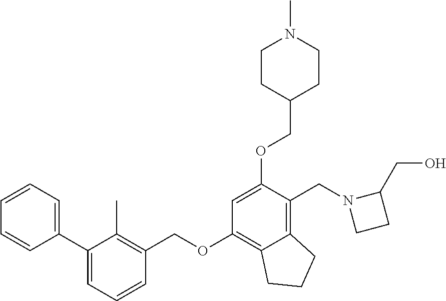 [SMILES: Cc1c(COc2cc(OCC3CCN(C)CC3)c(CN3CCC3CO)c3c2CCC3)cccc1-c1ccccc1] | |

### Example 78: Synthesis of (1-((7-((2-methyl-[1,1′-biphenyl]-3-yl)methoxy)-5-phenoxy-2,3-dihydro-1H-inden-4-yl)methyl)azetidin-2-yl)methanol

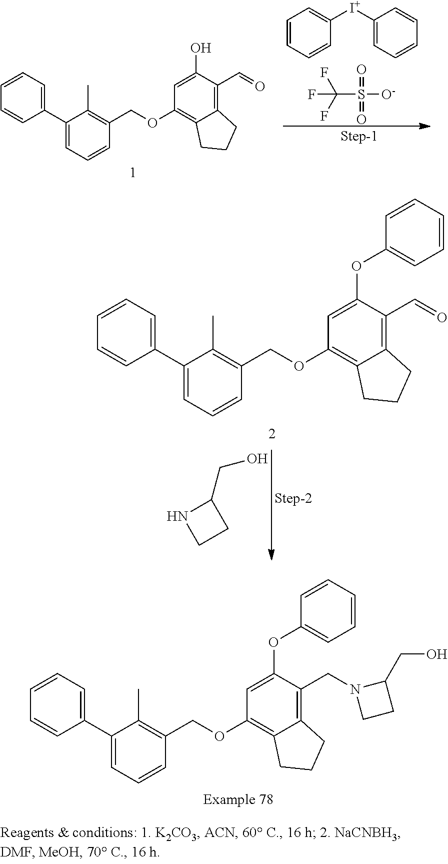 [SMILES: Cc1c(COc2cc(O)c(C=O)c3c2CCC3)cccc1-c1ccccc1 ; c1ccc([I+]c2ccccc2)cc1 ; O=S(=O)([O-])C(F)(F)F ; Cc1c(COc2cc(Oc3ccccc3)c(C=O)c3c2CCC3)cccc1-c1ccccc1 ; OCC1CCN1 ; Cc1c(COc2cc(Oc3ccccc3)c(CN3CCC3CO)c3c2CCC3)cccc1-c1ccccc1]

### Step-1: Synthesis of 7-((2-methyl-[1,1′-biphenyl]-3-yl) methoxy)-5-phenoxy-2,3-dihydro-1H-indene-4-carbaldehyde (2)

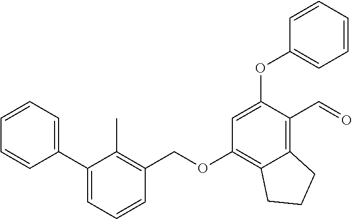 [SMILES: Cc1c(COc2cc(Oc3ccccc3)c(C=O)c3c2CCC3)cccc1-c1ccccc1]

To a stirred solution of 5-hydroxy-7-((2-methyl-[1,1′-biphenyl]-3-yl) methoxy)-2,3-dihydro-1H-indene-4-carbaldehyde (1, 1.0 g, 2.79 mmol) in acetonitrile (10 mL), potassium carbonate (1.15 g, 8.37 mmol) and diphenyl iodonium triflate (1.8 g, 4.18 mmol) were added under nitrogen atmosphere at room temperature. The reaction mixture was stirred for 16 h at 60° C. After completion of the reaction (monitored by TLC), the reaction mixture was concentrated, the residue was diluted with water (10 mL) and extracted with ethyl acetate (2×50 mL). The combined organic layer was dried over anhydrous sodium sulfate and concentrated under vacuum. The residue was purified by silica gel column chromatography using 5% methanol in dichloromethane to afford the desired product (2, 0.52 g, 46%) as brownish solid. LCMS (ES) m/z=435.2 [M+H]⁺.

### Step-2: Synthesis of (1-((7-((2-methyl-[1,1′-biphenyl]-3-yl) methoxy)-5-phenoxy-2,3-dihydro-1H-inden-4-yl) methyl) azetidin-2-yl) methanol (Example 78)

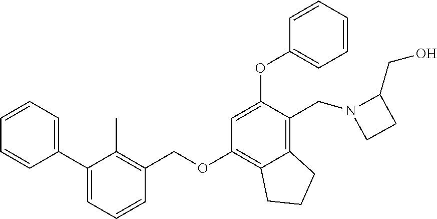 [SMILES: Cc1c(COc2cc(Oc3ccccc3)c(CN3CCC3CO)c3c2CCC3)cccc1-c1ccccc1]

To a stirred solution of 7-((2-methyl-[1,1′-biphenyl]-3-yl) methoxy)-5-phenoxy-2,3-dihydro-1H-indene-4-carbaldehyde (2, 0.5 g, 1.16 mmol) and azetidin-2-yl methanol (0.212 g, 1.74 mmol) in dimethylformamide (7 mL) and methanol (7 mL) was added acetic acid (0.348 g, 5.80 mmol) under nitrogen atmosphere at room temperature. The reaction mixture was stirred for 6 h at 70° C. and sodium cyanoborohydride (0.218 g, 3.48 mmol) was added. The reaction mixture was stirred at the same temperature for further 16 h. After completion of the reaction (monitored by TLC), the reaction mixture was diluted with water (10 mL) and extracted with 10% methanol in dichloromethane (3×50 mL). The organic layer was dried over anhydrous sodium sulfate and concentrated under vacuum. The resulting crude was purified by silica gel flash column chromatography using 10% methanol in dichloromethane as eluent to get title compound (Example 78, 0.013 g, 2.23%) as off white solid. LCMS (ES) m/z=506.38 [M+H]⁺; ¹H NMR (400 MHz, DMSO-d₆) δ ppm: 7.48-7.42 (m, 2H), 7.40-7.36 (m, 2H), 7.36-7.27 (m, 4H), 7.24 (t, J=7.6 Hz, 1H), 7.17 (d, J=7.6 Hz, 1H), 7.01 (t, J=7.2 Hz, 1H), 6.83 (d, J=8.0 Hz, 2H), 6.55 (s, 1H), 5.04 (s, 2H), 4.24 (bs, 1H), 3.47 (d, J=12 Hz, 1H), 3.38-3.30 (m, 1H), 3.28-3.20 (m, 2H), 3.15-3.08 (m, 1H), 3.06-2.98 (m, 2H), 2.95-2.88 (m, 1H), 2.81 (d, J=7.2 Hz, 2H), 2.74-2.69 (m, 1H), 2.13 (s, 3H), 2.07-1.95 (m, 2H), 1.87-1.78 (m, 1H), 1.72-1.65 (m, 1H). HPLC: 98.6%.

### Example 79: Synthesis of (1-((7-((2-methyl-[1,1′-biphenyl]-3-yl)methoxy)-5-(pyrazin-2-ylmethoxy)-2,3-dihydro-1H-inden-4-yl)methyl)azetidin-2-yl)methanol

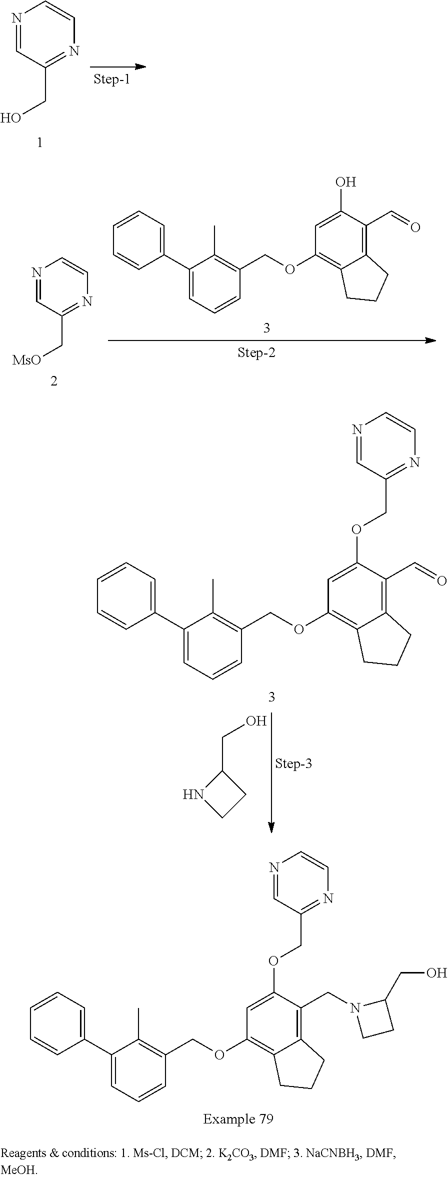

### Step-1: Synthesis of pyrazin-2-ylmethyl methanesulfonate (2)

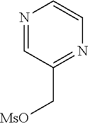 [SMILES: CS(=O)(=O)OCc1cnccn1]

To a solution of (pyrimidin-5-yl)methanol (0.1 g, 0.908 mmol) in dichloromethane (4 mL), triethylamine (0.28 g, 2.72 mmol) was added and after 10 min stirring, methane sulphonyl chloride (0.171 mL, 1.82 mmol) was added to it at 0° C. Progress of the reaction was monitored by LCMS and TLC. After completion of the reaction, the reaction mixture was diluted with water (20 mL) and extracted with dichloromethane (50 mL). The organic layer was concentrated to get the desired product (2, 0.120 g, crude) as brownish semi solid which was used for next step without further purification.

### Step 2: Synthesis of 7-((2-methyl-[1,1′-biphenyl]-3-yl) methoxy)-5-(pyrazin-2-ylmethoxy)-2,3-dihydro-1H-indene-4-carbaldehyde (3)

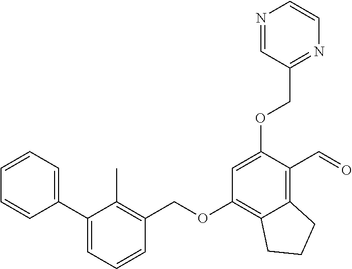 [SMILES: Cc1c(COc2cc(OCc3cnccn3)c(C=O)c3c2CCC3)cccc1-c1ccccc1]

To a solution of 5-hydroxy-7-((2-methyl-[1,1′-biphenyl]-3-yl) methoxy)-2,3-dihydro-1H-indene-4-carbaldehyde (0.14 g, 3.91 mmol) in N,N-dimethylformamide (20 mL), potassium carbonate (0.162 g, 1.17 mmol) and pyrazin-2-ylmethyl methanesulfonate (2, 0.120 g, 0.446 mmol) were sequentially added. The reaction mixture was stirred at room temperature for 16 h. After completion of the reaction (monitored by TLC), the reaction mixture was quenched with cold water (50 mL) and extracted with ethyl acetate (2×50 mL). The organic layer was dried (Na₂SO₄) and concentrated under reduced pressure. The resulting crude was purified by silica gel flash column chromatography to afford desired product (3, 0.150 g, 86% yield) as a brown solid. LCMS (ES) m/z=451.3 [M+H]⁺.

### Step 3: Synthesis of (1-((7-((2-methyl-[1,1′-biphenyl]-3-yl) methoxy)-5-(pyrazin-2-ylmethoxy)-2,3-dihydro-1H-inden-4-yl) methyl) azetidin-2-yl) methanol (Example 79)

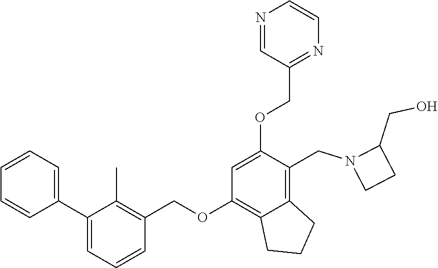 [SMILES: Cc1c(COc2cc(OCc3cnccn3)c(CN3CCC3CO)c3c2CCC3)cccc1-c1ccccc1]

To a solution of 7-((2-methyl-[1,1′-biphenyl]-3-yl) methoxy)-5-(pyrazin-2-ylmethoxy)-2,3-dihydro-1H-indene-4-carbaldehyde (0.14 g, 0.311 mmol) and azetidin-2-ylmethanol (0.027 g, 0.311 mmol) in N,N-dimethylformamide (7 mL) and methanol (7 mL), acetic acid (0.1 mL) was added at room temperature. The reaction mixture was stirred at 70° C. for 6 h and sodium cyanoborohydride (0.058 g, 0.93 mmol) was added to it. The reaction was further stirred for 16 h at the same temperature. After completion of the reaction (monitored by TLC), the reaction mixture was diluted with water (20 mL) and extracted with 10% methanol in dichloromethane (2×50 mL). The organic layer was dried (Na₂SO₄) and concentrated under reduced pressure. The residue was purified by silica gel column chromatography using 5% methanol in dichloromethane to get the title compound (Example 79, 0.025 g, 15% yield) as off white solid. LCMS (ES) m/z=522.35 [M+H]⁺. ¹H NMR (400 MHz, DMSO-d₆) δ ppm: 8.91 (s, 1H), 8.70-8.60 (m, 2H), 8.50-7.40 (m, 3H), 7.39-7.34 (m, 1H), 7.32 (d, J=7.2 Hz, 2H), 7.27 (t, J=7.6 Hz, 1H), 7.18 (d, J=7.6 Hz, 1H), 6.76 (s, 1H), 5.28 (s, 2H), 5.14 (s, 2H), 4.20 (bs, 1H), 3.57 (d, J=12.0 Hz, 1H), 3.49 (d, J=12.0 Hz, 1H), 3.25-3.35 (m, 3H), 3.10-3.00 (m, 1H), 2.98-2.80 (m, 3H), 2.75 (t, J=7.6 Hz, 2H), 2.21 (s, 3H), 2.00-1.92 (m, 2H), 1.90-1.80 (m, 1H), 1.75-1.67 (m, 1H). HPLC: 90.55%.

### Example 80: Synthesis of 1-((1-((5-((5-cyanopyridin-3-yl)methoxy)-7-((2-methyl-[1,1′-biphenyl]-3-yl)methoxy)-2,3-dihydro-1H-inden-4-yl)methyl)pyrrolidin-2-yl)methyl)-3-methylurea formate

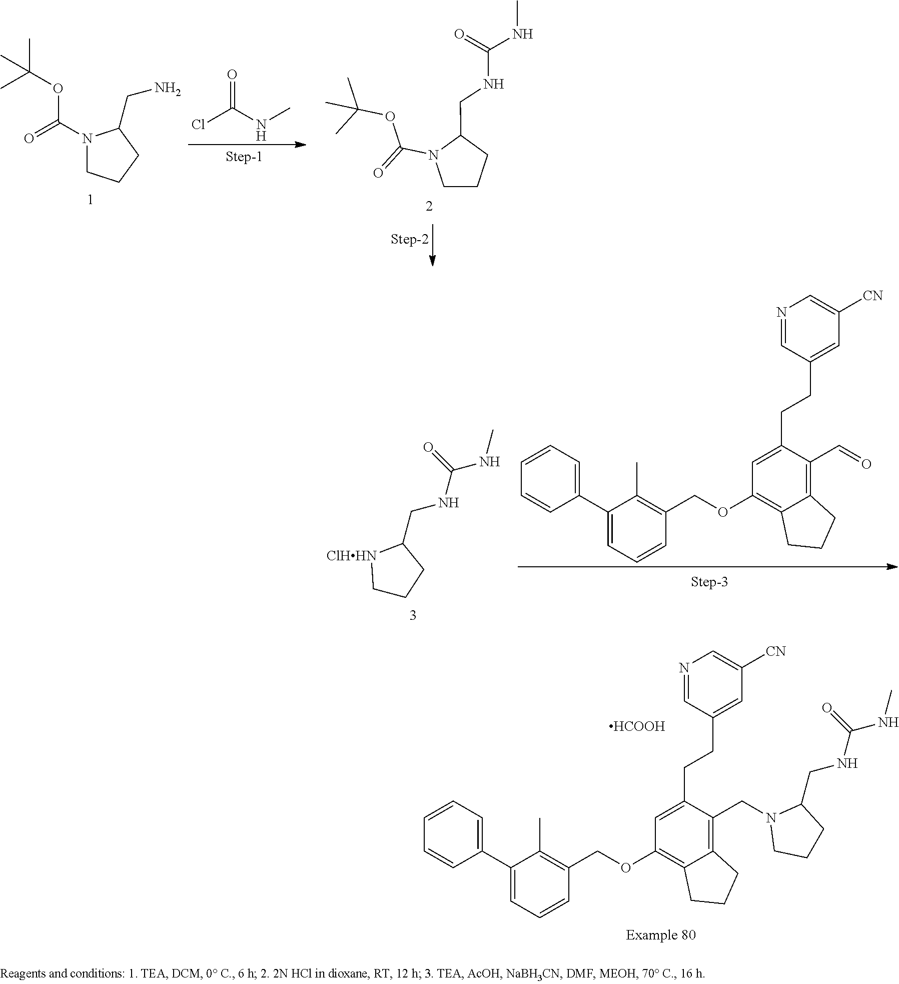

### Step-1: Preparation of tert-butyl 2-((3-methylureido)methyl)pyrrolidine-1-carboxylate (2)

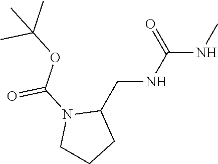 [SMILES: CNC(=O)NCC1CCCN1C(=O)OC(C)(C)C]

To a stirred solution of tert-butyl 2-(aminomethyl)pyrrolidine-1-carboxylate (1, 2 g, 10 mmol) in dichloromethane (20 mL) was added triethylamine (2.02 g, 20 mmol) and methylcarbamic chloride (1.12 g, 12 mmol) at 0° C. The resulting reaction mixture was stirred for further 6 h at the same temperature. After completion of the reaction as monitored by TLC, the reaction mixture was diluted with water (20 mL) and extracted with dichloromethane (2×20 mL). The combined organic layer was dried over sodium sulphate and concentrated under reduced pressure. The crude was purified by silica-gel column chromatography to obtained the desired product (2, 2.1 g, 81% yield) as wine colored oil. LCMS (ES) m/z=258.3 [M+H]⁺

### Step-2: Preparation of 1-methyl-3-(pyrrolidin-2-ylmethyl)urea hydrochloride (3)

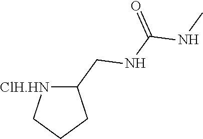

A solution of tert-butyl 2-((3-methylureido)methyl)pyrrolidine-1-carboxylate (2, 1 g, 3.8 mmol) in 2N hydrochloric acid solution in dioxane (10 mL) was stirred for 12 h at room temperature. After completion of the reaction as monitored by TLC, the reaction mixture was distilled under vacuum to obtained the desired product (3, 0.6 g, 80% yield) as white solid. LCMS (ES) m/z=158.1 [M+H]⁺

### Step-3: Preparation of 1-((1-((5-((5-cyanopyridin-3-yl)methoxy)-7-((2-methyl-[1,1′-biphenyl]-3-yl)methoxy)-2,3-dihydro-1H-inden-4-yl)methyl)pyrrolidin-2-yl)methyl)-3-methylurea formate (Example 80)

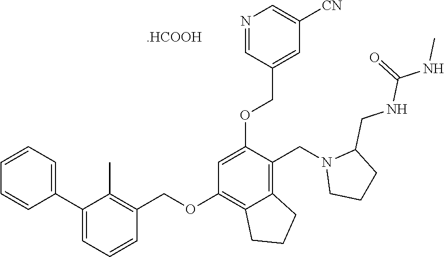 [SMILES: CNC(=O)NCC1CCCN1Cc1c(OCc2cncc(C#N)c2)cc(OCc2cccc(-c3ccccc3)c2C)c2c1CCC2 ; ".HCOOH"]

To a stirred solution of 5-(((4-formyl-7-((2-methyl-[1,1′-biphenyl]-3-yl)methoxy)-2,3-dihydro-1H-inden-5-yl)oxy)methyl)nicotinonitrile (0.4 g, 0.83 mmol) and 1-methyl-3-(pyrrolidin-2-ylmethyl)urea hydrochloride (3, 0.244 g, 1.26 mmol) in dimethylformamide (7 mL) and methanol (7 mL) was added triethylamine (0.34 g, 3.37 mmol) and acetic acid (0.253 g, 4.21 mmol) under nitrogen atmosphere at room temperature and stirred reaction mixture for 6 h at 70° C. To this reaction mass was added sodium cyanoborohydride (0.159 g, 2.53 mmol) and stirred at the same temperature for further 16 h. After completion of the reaction as monitored by TLC, the reaction mixture was diluted with water (10 mL) and extracted with 10% methanol in dichloromethane (3×15 mL). The organic layer was dried over anhydrous sodium sulfate and concentrated. The resulting crude was purified by silica gel column chromatography using 10% methanol in dichloromethane followed by prep HPLC to afford the title compound as white solid. LCMS (ES) m/z=616.34 [M+H]⁺; ¹H NMR (DMSO-d6, 400 MHz): δ 8.98 (d, J=1.6 Hz, 2H), 8.42 (bs, 1H), 8.14 (s, 1H), 7.49-7.42 (m, 3H), 7.38 (t, J=7.6 Hz, 1H), 7.31 (d, J=7.2 Hz, 2H), 7.27 (t, J=7.6 Hz, 1H), 7.18 (d, J=6.8 Hz, 1H), 6.76 (s, 1H), 5.92 (bs, 1H), 5.59 (bs, 1H), 5.30-5.20 (m, 2H), 5.15 (s, 2H), 3.83 (bs, 1H), 3.24 (bs, 1H), 3.00-2.80 (m, 3H), 2.80-2.70 (m, 3H), 2.45 (s, 3H), 2.21 (s, 3H), 2.22-1.93 (m, 3H), 1.83-1.75 (m, 1H), 1.63-1.35 (m, 3H). 2H merged with DMSO residual peak. HPLC: 95.32%.

### Biological Evaluation and Determination of Metabolic Stability:

### PD-L1 Enzyme Assay: Homogenous Time-Resolved Fluorescence (HTRF) Binding Assay

All binding studies were performed using PD-1/PD-L1 Binding Assay Kit from CisBio (Catalog #63ADK000CPAPEG), according to the manufacturer's protocol. The interaction between Tag1-PD-1 and Tag2-PD-1 was detected by anti-Tag1-Eu³⁺ (HTRF donor) and anti-Tag2-XL665 (HTRF acceptor). When the donor and acceptor antibodies were brought to close proximity due to PD-1 and PD-L1 binding, excitation of the donor antibody triggered fluorescent resonance energy transfer (FRET) towards the acceptor antibody, which in turn emitted specifically at 665 nm. This specific signal is positively proportional to PD-1/PD-L1 interaction. The compounds blocking PD-1/PD-L1 interaction will cause a reduction in HTRF signal. The necessary reagents were mixed in the following order: 2 μL compounds (or diluents buffer), 4 μL PD-L1 protein, 4 μL PD-1 protein. After an incubation of 15 minutes, 5 μL of anti-Tag1-Eu³⁺ and 5 μL of anti-Tag2-XL665 were added. The plate was sealed and incubated at room temperature for 1 h. The fluorescence emission was read at two different wavelengths (665 nm and 620 nm) on a BMG PheraStar® multi-plate reader.

Results were calculated from the 665 nm and 620 nm fluorescence signal and expressed in HTRF ratio=(665 nm/620 nm)×10⁴.

### Metabolic Stability in Liver Microsomes

The purpose of this experiment is to measure the metabolic half-life of NCEs in sub-cellular fractions such as human liver microsomes (HLM) or mouse liver microsomes (MLM). This provides an in vitro means to calculate intrinsic hepatic clearance, and to support the prediction of human pharmacokinetics. This approach has been successfully utilized at early phase of drug discovery projects, to provide SAR input for reducing metabolic instability and for predicting in vivo hepatic clearance.

Procedure: Potassium phosphate buffer (66.7 mM, pH 7.4) containing liver microsomes (mouse and human) (1.0 mg/mL) was pre-incubated separately with compound (1 μM) and positive control (verapamil, 1 μM) in a 37° C. water bath for 5 min. The reactions were initiated by adding 20 μL of 10 mM NADPH. Reactions without NADPH (0 and 30 min) were also incubated to rule out non-NADPH metabolism or chemical instability in the incubation buffer. All reactions were terminated using 200 μL of ice-cold acetonitrile containing internal standard at 0, 5, 15 and 30 min. The vials were centrifuged at 3000 rpm for 15 min. The supernatants thus obtained were analyzed on LC-MS/MS to monitor the disappearance of test compound.

### Animal Experiments Details

Institutional Animal Ethical Committee (IAEC) of Jubilant Biosys (IAEC/JDC/2019/188R (for Mice) and IAEC/JDC/2019/189R (for Rat) nominated by CPCSEA (Committee for the Purpose of Control and Supervision of Experiments on Animals) approved the mice and rat pharmacokinetic experiments. Male Balb/c mice (˜6-8 weeks old with body weight range of 22-25 g) and male SD rats (6-8 weeks old with body weight range of 200-250 g) were procured from Vivo Biotech, Hyderabad, India. Animals were quarantined in Jubilant Biosys Animal House for a period of 7 days with a 12:12 h light: dark cycles, and prior to the study the animals were stratified as per body weight.

Housing: The animals were group housed in standard polycarbonate cages, with stainless steel top grill where pelleted food and drinking water bottle are placed; corn cob was used as bedding material and changed at least twice a week or as required.

Diet ad libitum: Rodent feed manufactured by Altromin Spezialfutter GmbH & Co. KG., ImSeelenkamp20. D-32791 Lage, was provided.

Water ad libitum: Purified water was provided ad libitum to animals in polycarbonate bottles with stainless steel sipper tubes.

### Pharmacokinetics Studies

Procedure for Mice: Intravenous pharmacokinetics study was done at doses of 5, 10 mg/kg respectively at dose volume 5 mL/kg for IV route. Sparse sampling was done and at each time point three mice were used for blood sampling (˜100 μL) were collected from retro-orbital plexus at 0.083 (Only for IV) and 24 h. Blood samples collected in tubes containing K2 EDTA as anticoagulant and centrifuged for 5 min at 10,000 rpm in a refrigerated centrifuge (Biofuge, Heraeus, Germany) maintained at 4° C. for plasma separation. Group I (IV) received compound by intravenously by tail vein at 2 mg/Kg in solution formulation. Blood concentration-time data of compound was analyzed by non-compartmental method using Phoenix WinNonlin Version 8.1.

### Brain Exposure Study in Mice

The mice were placed in isoflurane anesthesia chamber; following complete anesthesia (3-5% isoflurane) blood sample (0.5 mL) was collected from retro-orbital plexus using mice capillary.

The mice were sacrificed by cervical dislocation. The dorsal surface of the skull was peeled away from the brain using bone cutter, dura matter was gently removed from surface of brain with help of forceps. Brain was gently taken out away from the head and placed in PBS buffer to remove blood. Brain was placed on blotting paper to remove the blood spots and transferred in pre-labelled tube. The isolated brain was weighed and homogenised using 5× volume of phosphate buffer saline (pH-7.4). During homogenization the brain homogenate was kept in ice till sample processing. The homogenate was processed by specified extraction process and analyzed by LC-MS/MS.

### Evaluation of Biological Activity and Metabolic Stability:

Table 8 below, shows the biological activity of compounds of the present invention in PD1/PD-L1 inhibition assay. Compounds having IC₅₀<100 nM are designated as “A”; 100-500 nM are designated as “B”; and >500 nM are designated as “C” respectively.

TABLE 8

| | | HLM/ |
|---|---|---|
| | PD1/ | MLM % |
| Example | PD-L1 | remaining |
| No. | Activity | @ 30 min |
| 1 | A | 72/83 |
| 2 | A | 58/62 |
| 3 | A | 22/14 |
| 4 | A | 20/20 |
| 5 | A | 16/13 |
| 6 | A | 67/87 |
| 7 | A | 66/76 |
| 8 | A | 61/85 |
| 9 | A | 8/21 |
| 10 | A | 69/70 |
| 11 | A | 21/11 |
| 12 | B | ND |
| 13 | A | ND |
| 14 | A | ND |
| 15 | A | ND |
| 16 | A | ND |
| 17 | A | ND |
| 18 | A | ND |
| 19 | A | ND |
| 20 | A | ND |
| 21 | A | ND |
| 22 | A | ND |
| 23 | A | 27/25 |
| 24 | A | 72/67 |
| 25 | A | 45/61 |
| 26 | A | 6/5 |
| 27 | A | 91/84 |
| 28 | A | 74/82 |
| 29 | A | 56/71 |
| 30 | A | 57/74 |
| 31 | A | 65/79 |
| 32 | A | 81/84 |
| 33 | B | ND |
| 34 | A | 8/5 |
| 35 | A | 3/3 |
| 36 | B | ND |
| 37 | A | 86/88 |
| 38 | A | 78/84 |
| 39 | B | ND |
| 40 | B | ND |
| 41 | B | ND |
| 42 | A | ND |
| 43 | B | ND |
| 44 | A | 33/15 |
| 45 | A | ND |
| 46 | B | ND |
| 47 | B | ND |
| 48 | B | ND |
| 49 | A | ND |
| 50 | B | ND |
| 51 | B | ND |
| 52 | A | ND |
| 53 | A | ND |
| 54 | C | ND |
| 55 | B | ND |
| 56 | B | ND |
| 57 | C | ND |
| 58 | C | ND |
| 59 | C | ND |
| 60 | B | ND |
| 61 | C | ND |
| 62 | A | ND |
| 63 | C | ND |
| 64 | A | ND |
| 65 | A | ND |
| 66 | C | ND |
| 67 | A | ND |
| 68 | C | ND |
| 69 | C | ND |
| 70 | C | ND |
| 71 | B | ND |
| 72 | B | ND |
| 73 | B | ND |
| 74 | C | ND |
| 75 | C | ND |
| 76 | B | ND |
| 78 | C | ND |
| 80 | A | ND |

### Evaluation of Brain Exposure Data by IV Route

TABLE 9

| | | | Brain | | Plasma | | Brain/ |
|---|---|---|---|---|---|---|---|
| | Time | Dose | (ng/g) | | (ng/mL) | | Plasma |
| Example | (h) | (mpk) | Mean | SD | Mean | SD | ratio |
| 27 | 0.5 | 10 | 11569 | 4743 | 949 | 82 | 12.2 |
| 31 | 0.5 | 5 | 2449 | 180 | 501 | 63 | 4.9 |

The above mentioned compounds have potential to be developed as drugs to alleviate the PD1/PD-L1 activity and thus treating cancer, and other diseases or conditions associated with activation of PD1/PD-L1.

1. A compound of Formula (I): 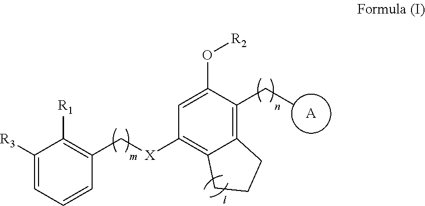
their stereoisomers, an N-oxide or pharmaceutically acceptable salts thereof,
wherein,
X is selected from O or NR′;
Ring A is selected from C₆₋₁₀ aryl, C₃₋₁₀ cycloalkyl, C₇₋₁₆ alkylaryl, C₂₋₁₀ heteroaryl, C₂₋₂₀ heterocyclyl, —CO—C₂-20 heterocyclyl, or —C(O)NR₄-C₂₋₂₀ heterocyclyl; wherein, C₆₋₁₀ aryl, C₃₋₁₀ cycloalkyl, C₇₋₁₆ alkylaryl, C₂₋₁₀ heteroaryl, C₂₋₂₀ heterocyclyl, —CO—C₂₋₂₀ heterocyclyl, or —C(O)NR₄-C₂₋₂₀ heterocyclyl is optionally substituted with one or more groups selected from halogen, hydroxy, C₁₋₁₀ alkyl, C₁₋₁₀ alkoxy, C₁₋₁₀ haloalkyl, C₂₋₁₀ alkylalkoxy, —CH₂—NRₐC(O)R_{b}, —CRₐR_{b}—OR_{c}, —CRₐR_{b}—NR_{c}Rₐ, or —CH₂—NHC(O)NRₐR_{b}; wherein, Rₐ, R_{b}, R_{c}, and R_{d} are independently selected from hydrogen, halogen, C₁₋₁₀ alkyl, —C(O)R″, C₃₋₁₀ cycloalkyl, C₁₋₁₀ haloalkyl, or C₁₋₁₀ alkoxy;
R′ is selected from hydrogen or C₁₋₁₀ alkyl;
R₁ is selected from hydrogen, cyano or C₁₋₁₀ alkyl;
R₂ is selected from hydrogen, C₁₋₁₀ alkyl, C₆₋₁₀ aryl, C₃₋₁₀ cycloalkyl, C₁₋₁₀ haloalkyl, C₇₋₁₆ alkylaryl, C₂₋₁₀ heteroaryl, C₃₋₂₀ alkyl heteroaryl, C₂₋₂₀ heterocyclyl, or C₃₋₂₀ alkyl heterocyclyl; wherein, C₁₋₁₀ alkyl, C₆₋₁₀ aryl, C₃₋₁₀ cycloalkyl, C₁₋₁₀ haloalkyl, C₇₋₁₆ alkylaryl, C₂₋₁₀ heteroaryl, C₃₋₂₀ alkyl heteroaryl, C₂₋₂₀ heterocyclyl, or C₃₋₂₀ alkyl heterocyclyl, is optionally substituted with one or more groups selected from halogen, cyano, hydroxy, —C(O)NH₂, C₁₋₁₀ alkyl, or C₆₋₁₀ aryl;
R₃ is selected from halogen, C₆₋₁₀ aryl, or C₂₋₁₀ heteroaryl; wherein, C₆₋₁₀ aryl, or C₂₋₁₀ heteroaryl, is optionally substituted with one or more groups selected from halogen, haloalkyl, cyano, hydroxy, amino, C₁₋₁₀ alkyl, OR″, C₆₋₁₀ aryl, C₂₋₂₀ heterocyclyl, or C₂₋₁₀ heteroaryl;
R″ is selected from hydrogen, halogen, C₁₋₁₀ alkyl, or C₁₋₁₀ haloalkyl;
R₄ is selected from hydrogen or C₁₋₁₀ alkyl;
m is 1 to 5; n is 0 to 5; and l is 1 to 5,
provided that the compound of Formula (I) is not:

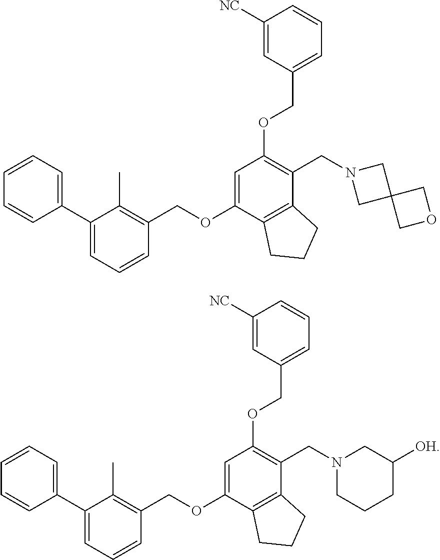 [SMILES: Cc1c(COc2cc(OCc3cccc(C#N)c3)c(CN3CC4(COC4)C3)c3c2CCC3)cccc1-c1ccccc1 ; Cc1c(COc2cc(OCc3cccc(C#N)c3)c(CN3CCCC(O)C3)c3c2CCC3)cccc1-c1ccccc1]

2. The compound as claimed in claim 1, having compound of Formula (IA): 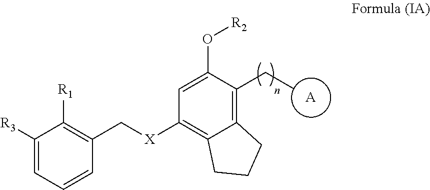
their stereoisomers, an N-oxide or pharmaceutically acceptable salts thereof;
wherein,
X is selected from O;
Ring A is selected from C₂₋₂₀ heterocyclyl, —CO—C₂₋₂₀ heterocyclyl, or —C(O)NR₄-C₂₋₂₀ heterocyclyl; wherein, C₂₋₂₀ heterocyclyl, —CO—C₂₋₂₀ heterocyclyl, or —C(O)NR₄-C₂₋₂₀ heterocyclyl is optionally substituted with one or more groups selected from halogen, hydroxy, C₁₋₁₀ alkyl, —CH₂—NRₐC(O)R_{b}, —CRₐR_{b}—OR_{c}, —CRₐR_{b}—NR_{c}R_{d}, or —CH₂—NHC(O)NRₐR_{b}; wherein, Rₐ, R_{b}, R_{c}, and R_{d} are independently selected from hydrogen, C₁₋₁₀ alkyl, or —C(O)R″;
R₁ is selected from cyano or C₁₋₁₀ alkyl;
R₂ is selected from C₆₋₁₀ aryl, C₁₋₁₀ haloalkyl, C₇₋₁₆ alkylaryl, C₃₋₂₀ alkyl heteroaryl or C₃₋₂₀ alkyl heterocyclyl; wherein, C₆₋₁₀ aryl, C₁₋₁₀ haloalkyl, C₇₋₁₆ alkylaryl, C₃₋₂₀ alkyl heteroaryl, or C₃₋₂₀ alkyl heterocyclyl, is optionally substituted with one or more groups selected from cyano, hydroxy, —C(O)NH₂, or C₁₋₁₀ alkyl;
R₃ is selected from halogen, C₆₋₁₀ aryl, or C₂₋₁₀ heteroaryl; wherein, C₆₋₁₀ aryl, or C₂₋₁₀ heteroaryl, is optionally substituted with one or more groups selected from halogen, haloalkyl, hydroxy, amino, C₁₋₁₀ alkyl, OR″ or C₂₋₂₀ heterocyclyl;
R″ is selected from C₁₋₁₀ alkyl, or C₁₋₁₀ haloalkyl;
R₄ is hydrogen; and
n is 0 to 1.

3. The compound as claimed in claim 1, having compound of Formula (IB): 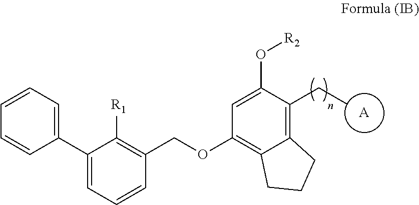
their stereoisomers, an N-oxide or pharmaceutically acceptable salts thereof;
wherein,
Ring A is selected from C₆₋₁₀ aryl, C₃₋₁₀ cycloalkyl, C₇₋₁₆ alkylaryl, C₂₋₁₀ heteroaryl, C₂₋₂₀ heterocyclyl, —CO—C₂-20 heterocyclyl, or —C(O)NR₄-C₂₋₂₀ heterocyclyl; wherein, C₆₋₁₀ aryl, C₃₋₁₀ cycloalkyl, C₇₋₁₆ alkylaryl, C₂₋₁₀ heteroaryl, C₂₋₂₀ heterocyclyl, —CO—C₂₋₂₀ heterocyclyl, or —C(O)NR₄-C₂₋₂₀ heterocyclyl is optionally substituted with one or more groups selected from halogen, hydroxy, C₁₋₁₀ alkyl, C₁₋₁₀ alkoxy, C₁₋₁₀ haloalkyl, C₂₋₁₀ alkylalkoxy, —CH₂—NRₐC(O)R_{b}, —CRₐR_{b}—OR_{c}, —CRₐR_{b}—NR_{c}R_{d}, or —CH₂—NHC(O)NRₐR_{b}; wherein, Rₐ, R_{b}, R_{c}, and R_{d} are independently selected from hydrogen, halogen, C₁₋₁₀ alkyl, —C(O)R″, C₃₋₁₀ cycloalkyl, C₁₋₁₀ haloalkyl, or C₁₋₁₀ alkoxy;
R₁ is selected from hydrogen, cyano or C₁₋₁₀ alkyl;
R₂ is selected from hydrogen, C₁₋₁₀ alkyl, C₆₋₁₀ aryl, C₃₋₁₀ cycloalkyl, C₁₋₁₀ haloalkyl, C₇₋₁₆ alkylaryl, C₂₋₁₀ heteroaryl, C₃₋₂₀ alkyl heteroaryl, C₂₋₂₀ heterocyclyl, or C₃₋₂₀ alkyl heterocyclyl; wherein, C₁₋₁₀ alkyl, C₆₋₁₀ aryl, C₃₋₁₀ cycloalkyl, C₁₋₁₀ haloalkyl, C₇₋₁₆ alkylaryl, C₂₋₁₀ heteroaryl, C₃₋₂₀ alkyl heteroaryl, C₂₋₂₀ heterocyclyl, or C₃₋₂₀ alkyl heterocyclyl, is optionally substituted with one or more groups selected from halogen, cyano, hydroxy, —C(O)NH₂, C₁₋₁₀ alkyl, or C₆₋₁₀ aryl;
R″ is selected from hydrogen, halogen, C₁₋₁₀ alkyl, or C₁₋₁₀ haloalkyl;
R₄ is selected from hydrogen or C₁₋₁₀ alkyl; and
n is 0 to 1.

4. The compound as claimed in claim 1, having compound of Formula (IC): 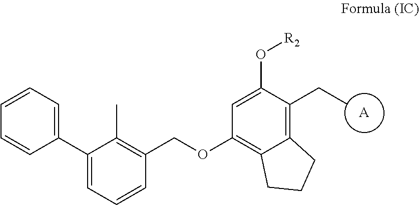
their stereoisomers, an N-oxide or pharmaceutically acceptable salts thereof;
wherein,
Ring A is selected from C₆₋₁₀ aryl, C₃₋₁₀ cycloalkyl, C₇₋₁₆ alkylaryl, C₂₋₁₀ heteroaryl, C₂₋₂₀ heterocyclyl, —CO—C₂-20 heterocyclyl, or —C(O)NR₄-C₂₋₂₀ heterocyclyl; wherein, C₆₋₁₀ aryl, C₃₋₁₀ cycloalkyl, C₇₋₁₆ alkylaryl, C₂₋₁₀ heteroaryl, C₂₋₂₀ heterocyclyl, —CO—C₂₋₂₀ heterocyclyl, or —C(O)NR₄-C₂₋₂₀ heterocyclyl is optionally substituted with one or more groups selected from halogen, hydroxy, C₁₋₁₀ alkyl, C₁₋₁₀ alkoxy, C₁₋₁₀ haloalkyl, C₂₋₁₀ alkylalkoxy, —CH₂—NRₐC(O)R_{b}, —CRₐR_{b}—OR_{c}, —CRₐR_{b}—NR_{c}Rₐ, or —CH₂—NHC(O)NRₐR_{b}; wherein, Rₐ, R_{b}, R_{c}, and R_{d} are independently selected from hydrogen, halogen, C₁₋₁₀ alkyl, —C(O)R″, C₃₋₁₀ cycloalkyl, C₁₋₁₀ haloalkyl, or C₁₋₁₀ alkoxy;
R₂ is selected from hydrogen, C₁₋₆ alkyl, C₆₋₁₀ aryl, C₃₋₁₀ cycloalkyl, C₁₋₆ haloalkyl, C₇₋₁₆ alkylaryl, C₂₋₁₀ heteroaryl, C₃₋₂₀ alkyl heteroaryl, C₂₋₂₀ heterocyclyl, or C₃₋₂₀ alkyl heterocyclyl; wherein, C₁₋₆ alkyl, C₆₋₁₀ aryl, C₃₋₁₀ cycloalkyl, C₁₋₆ haloalkyl, C₇₋₁₆ alkylaryl, C₂₋₁₀ heteroaryl, C₃₋₂₀ alkyl heteroaryl, C₂₋₂₀ heterocyclyl, or C₃₋₂₀ alkyl heterocyclyl, is optionally substituted with one or more groups selected from halogen, cyano, hydroxy, —C(O)NH₂, C₁₋₁₀ alkyl, or C₆₋₁₀ aryl;
R″ is selected from hydrogen, halogen, C₁₋₁₀ alkyl, or C₁₋₁₀ haloalkyl; and
R₄ is selected from hydrogen or C₁₋₆ alkyl.

5. The compound as claimed in claim 1, their stereoisomers, an N-oxide or pharmaceutically acceptable salts thereof, wherein A is selected from: 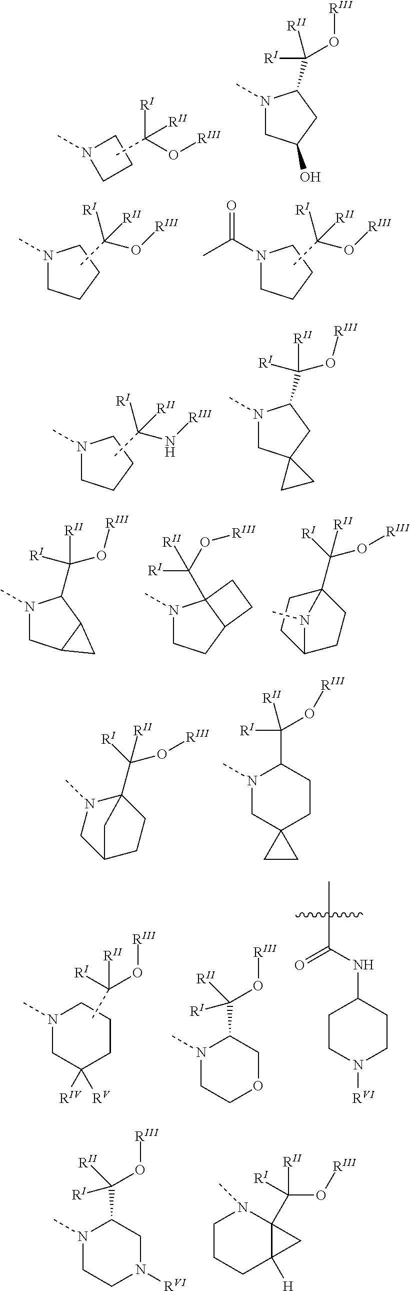 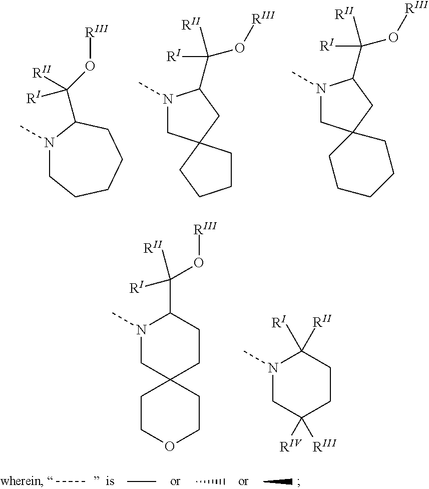
R^{I}, R^{II}, R^{III}, R^{IV}, R^{V} and R^{VI} are independently selected from hydrogen, C₁₋₁₀ alkyl, —C(O)R″, —C(O)NH—R″, —CH₂—OR″, halogen or C₁₋₁₀ haloalkyl.

6. The compound as claimed in claim 1, wherein the compound is selected from:

| Example No. | IUPAC Name |
|---|---|
| 1 | (S)-5-(((4-((2-(hydroxymethyl)piperidin-1-yl)methyl)-7-((2- |
| | methyl-[1,1′-biphenyl]-3-yl)methoxy)-2,3-dihydro-1H-inden-5- |
| | yl)oxy)methyl)nicotinonitrile; |
| 2 | (S)-3-(((4-((2-(hydroxymethyl)piperidin-1-yl)methyl)-7-((2- |
| | methyl-[1,1′-biphenyl]-3-yl)methoxy)-2,3-dihydro-1H-inden-5- |
| | yl)oxy)methyl)benzonitrile; |
| 3 | (S)-5-(((7-((2-cyano-[1,1′-biphenyl]-3-yl)methoxy)-4-((2- |
| | (hydroxymethyl)pyrrolidin-1-yl)methyl)-2,3-dihydro-1H-inden- |
| | 5-yl)oxy)methyl)nicotinonitrile; |
| 4 | (S)-3-(((6-((3-cyanobenzyl)oxy)-7-((2- |
| | (hydroxymethyl)pyrrolidin-1-yl)methyl)-2,3-dihydro-1H-inden- |
| | 4-yl)oxy)methyl)-[1,1′-biphenyl]-2-carbonitrile; |
| 5 | (S)-5-(((7-((2-cyano-[1,1′-biphenyl]-3-yl)methoxy)-4-((2- |
| | (hydroxymethyl)piperidin-1-yl)methyl)-2,3-dihydro-1H-inden-5- |
| | yl)oxy)methyl)nicotinonitrile; |
| 6 | (5-(((4-(((2S,4R)-4-hydroxy-2-(hydroxymethyl)pyrrolidin-1- |
| | yl)methyl)-7-((2-methyl-[1,1′-biphenyl]-3-yl)methoxy)-2,3- |
| | dihydro-1H-inden-5-yl)oxy)methyl)nicotinonitrile; |
| 7 | (S)-3-(((4-((2-(hydroxymethyl)pyrrolidin-1-yl)methyl)-7-((2- |
| | methyl-[1,1′-biphenyl]-3-yl)methoxy)-2,3-dihydro-1H-inden-5- |
| | yl)oxy)methyl)benzonitrile; |
| 8 | (S)-5-(((4-((2-(hydroxymethyl)pyrrolidin-1-yl)methyl)-7-((2- |
| | methyl-[1,1′-biphenyl]-3-yl)methoxy)-2,3-dihydro-1H-inden-5- |
| | yl)oxy)methyl)nicotinonitrile; |
| 9 | (R)-5-(((4-((3-(hydroxymethyl)morpholino)methyl)-7-((2- |
| | methyl-[1,1′-biphenyl]-3-yl)methoxy)-2,3-dihydro-1H-inden-5- |
| | yl)oxy)methyl)nicotinonitrile; |
| 10 | 5-(((4-((2-(hydroxymethyl)azetidin-1-yl)methyl)-7-((2-methyl- |
| | [1,1′-biphenyl]-3-yl)methoxy)-2,3-dihydro-1H-inden-5- |
| | yl)oxy)methyl)nicotinonitrile; |
| 11 | 5-(((4-((6-(hydroxymethyl)-5-azaspiro[2.5]octan-5-yl)methyl)-7- |
| | ((2-methyl-[1,1′-biphenyl]-3-yl)methoxy)-2,3-dihydro-1H-inden- |
| | 5-yl)oxy)methyl)nicotinonitrile; |
| 12 | 5-(((4-((5,5-difluoro-2-(hydroxymethyl)piperidin-1-yl)methyl)-7- |
| | ((2-methyl-[1,1′-biphenyl]-3-yl)methoxy)-2,3-dihydro-1H-inden- |
| | 5-yl)oxy)methyl)nicotinonitrile; |
| 13 | 5-(((4-((2-(hydroxymethyl)-4-methylpiperazin-1-yl)methyl)-7- |
| | ((2-methyl-[1,1′-biphenyl]-3-yl)methoxy)-2,3-dihydro-1H-inden- |
| | 5-yl)oxy)methyl)nicotinonitrile; |
| 14 | 5-(((4-((3-(hydroxymethyl)azetidin-1-yl)methyl)-7-((2-methyl- |
| | [1,1′-biphenyl]-3-yl)methoxy)-2,3-dihydro-1H-inden-5- |
| | yl)oxy)methyl)nicotinonitrile; |
| 15 | (R)-5-(((4-((3-(hydroxymethyl)pyrrolidin-1-yl)methyl)-7-((2- |
| | methyl-[1,1′-biphenyl]-3-yl)methoxy)-2,3-dihydro-1H-inden-5- |
| | yl)oxy)methyl)nicotinonitrile; |
| 16 | (S)-5-(((4-((3-(hydroxymethyl)piperidin-1-yl)methyl)-7-((2- |
| | methyl-[1,1′-biphenyl]-3-yl)methoxy)-2,3-dihydro-1H-inden-5- |
| | yl)oxy)methyl)nicotinonitrile; |
| 17 | 5-(((4-((4-(hydroxymethyl)piperidin-1-yl)methyl)-7-((2-methyl- |
| | [1,1′-biphenyl]-3-yl)methoxy)-2,3-dihydro-1H-inden-5- |
| | yl)oxy)methyl)nicotinonitrile; |
| 18 | (S)-5-(((4-((3-(hydroxymethyl)pyrrolidin-1-yl)methyl)-7-((2- |
| | methyl-[1,1′-biphenyl]-3-yl)methoxy)-2,3-dihydro-1H-inden-5- |
| | yl)oxy)methyl)nicotinonitrile; |
| 19 | (R)-5-(((4-((2-(hydroxymethyl)pyrrolidin-1-yl)methyl)-7-((2- |
| | methyl-[1,1′-biphenyl]-3-yl)methoxy)-2,3-dihydro-1H-inden-5- |
| | yl)oxy)methyl)nicotinonitrile; |
| 20 | 5-(((4-((2-(hydroxymethyl)azepan-1-yl)methyl)-7-((2-methyl- |
| | [1,1′-biphenyl]-3-yl)methoxy)-2,3-dihydro-1H-inden-5- |
| | yl)oxy)methyl)nicotinonitrile; |
| 21 | (R)-5-(((4-((3-(hydroxymethyl)piperidin-1-yl)methyl)-7-((2- |
| | methyl-[1,1′-biphenyl]-3-yl)methoxy)-2,3-dihydro-1H-inden-5- |
| | yl)oxy)methyl)nicotinonitrile 2,2,2-trifluoroacetate; |
| 22 | 5-(((4-((2,2-bis(hydroxymethyl)piperidin-1-yl)methyl)-7-((2- |
| | methyl-[1,1′-biphenyl]-3-yl)methoxy)-2,3-dihydro-1H-inden-5- |
| | yl)oxy)methyl)nicotinonitrile 2,2,2-trifluoroacetate; |
| 23 | (S)-3-(((7-((2-(hydroxymethyl)piperidin-1-yl)methyl)-6-(2,2,2- |
| | trifluoroethoxy)-2,3-dihydro-1H-inden-4-yl)oxy)methyl)-[1,1′- |
| | biphenyl]-2-carbonitrile; |
| 24 | (S)-(1-((7-((2-methyl-[1,1′-biphenyl]-3-yl)methoxy)-5-(2,2,2- |
| | trifluoroethoxy)-2,3-dihydro-1H-inden-4-yl)methyl)piperidin-2- |
| | yl)methanol; |
| 25 | (S)-5-(((4-((6-(hydroxymethyl)-5-azaspiro[2.4]heptan-5- |
| | yl)methyl)-7-((2-methyl-[1,1′-biphenyl]-3-yl)methoxy)-2,3- |
| | dihydro-1H-inden-5-yl)oxy)methyl)nicotinonitrile; |
| 26 | (S)-5-(((7-((2-cyano-[1,1′-biphenyl]-3-yl)methoxy)-4-((6- |
| | (hydroxymethyl)-5-azaspiro[2.4]heptan-5-yl)methyl)-2,3- |
| | dihydro-1H-inden-5-yl)oxy)methyl)nicotinonitrile; |
| 27 | (S)-(1-((5-(2-fluoroethoxy)-7-((2-methyl-[1,1′-biphenyl]-3- |
| | yl)methoxy)-2,3-dihydro-1H-inden-4-yl)methyl)piperidin-2- |
| | yl)methanol; |
| 28 | (S)-(1-((5-(2-fluoroethoxy)-7-((2-methyl-[1,1′-biphenyl]-3- |
| | yl)methoxy)-2,3-dihydro-1H-inden-4-yl)methyl)pyrrolidin-2- |
| | yl)methanol; |
| 29 | (S)-3-(((6-(2-fluoroethoxy)-7-((2-(hydroxymethyl)piperidin-1- |
| | yl)methyl)-2,3-dihydro-1H-inden-4-yl)oxy)methyl)-[1,1′- |
| | biphenyl]-2-carbonitrile; |
| 30 | (S)-(1-((5-((5-fluoropentyl)oxy)-7-((2-methyl-[1,1′-biphenyl]-3- |
| | yl)methoxy)-2,3-dihydro-1H-inden-4-yl)methyl)piperidin-2- |
| | yl)methanol; |
| 31 | (S)-(1-((5-(4-fluorobutoxy)-7-((2-methyl-[1,1′-biphenyl]-3- |
| | yl)methoxy)-2,3-dihydro-1H-inden-4-yl)methyl)piperidin-2- |
| | yl)methanol; |
| 32 | (S)-(1-((5-(3-fluoropropoxy)-7-((2-methyl-[1,1′-biphenyl]-3- |
| | yl)methoxy)-2,3-dihydro-1H-inden-4-yl)methyl)piperidin-2- |
| | yl)methanol |
| 33 | (S)-(1-((5-(4-fluorobutoxy)-7-((2-methyl-[1,1′-biphenyl]-3- |
| | yl)methoxy)-2,3-dihydro-1H-inden-4-yl)methyl)azetidin-2- |
| | yl)methanol; |
| 34 | 3-(((4-((1-(hydroxymethyl)-2-azabicyclo[4.1.0]heptan-2- |
| | yl)methyl)-7-((2-methyl-[1,1′-biphenyl]-3-yl)methoxy)-2,3- |
| | dihydro-1H-inden-5-yl)oxy)methyl)benzonitrile; |
| 35 | 5-(((4-((1-(hydroxymethyl)-2-azabicyclo[4.1.0]heptan-2- |
| | yl)methyl)-7-((2-methyl-[1,1′-biphenyl]-3-yl)methoxy)-2,3- |
| | dihydro-1H-inden-5-yl)oxy)methyl)nicotinonitrile; |
| 36 | 3-(((6-((3-cyanobenzyl)oxy)-7-((1-(hydroxymethyl)-2- |
| | azabicyclo[4.1.0]heptan-2-yl)methyl)-2,3-dihydro-1H-inden-4- |
| | yl)oxy)methyl)-[1,1′-biphenyl]-2-carbonitrile; |
| 37 | (S)-5-(((4-((2-(hydroxymethyl)piperidin-1-yl)methyl)-7-((2- |
| | methyl-[1,1′-biphenyl]-3-yl)methoxy)-2,3-dihydro-1H-inden-5- |
| | yl)oxy)methyl)nicotinamide; |
| 38 | (S)-3-(((4-((2-(hydroxymethyl)pyrrolidin-1-yl)methyl)-7-((2- |
| | methyl-[1,1′-biphenyl]-3-yl)methoxy)-2,3-dihydro-1H-inden-5- |
| | yl)oxy)methyl)benzamide; |
| 39 | (S)-(1-((5-((1-methyl-1H-pyrazol-4-yl)methoxy)-7-((2-methyl- |
| | [1,1′-biphenyl]-3-yl)methoxy)-2,3-dihydro-1H-inden-4- |
| | yl)methyl)piperidin-2-yl)methanol; |
| 40 | (1-((7-((2-methyl-[1,1′-biphenyl]-3-yl)methoxy)-5-(thiazol-4- |
| | ylmethoxy)-2,3-dihydro-1H-inden-4-yl)methyl)azetidin-2- |
| | yl)methanol; |
| 41 | (1-((5-((1-methyl-1H-imidazol-4-yl)methoxy)-7-((2-methyl- |
| | [1,1′-biphenyl]-3-yl)methoxy)-2,3-dihydro-1H-inden-4- |
| | yl)methyl)azetidin-2-yl)methanol; |
| 42 | (1-((7-((2-methyl-[1,1′-biphenyl]-3-yl)methoxy)-5-(pyrimidin-5- |
| | ylmethoxy)-2,3-dihydro-1H-inden-4-yl)methyl)azetidin-2- |
| | yl)methanol; |
| 43 | (1-((7-((2-methyl-[1,1′-biphenyl]-3-yl)methoxy)-5-(oxazol-4- |
| | ylmethoxy)-2,3-dihydro-1H-inden-4-yl)methyl)azetidin-2- |
| | yl)methanol; |
| 44 | (S)-3-cyano-5-(((4-((2-(hydroxymethyl)pyrrolidin-1-yl)methyl)- |
| | 7-((2-methyl-[1,1′-biphenyl]-3-yl)methoxy)-2,3-dihydro-1H- |
| | inden-5-yl)oxy)methyl)pyridine 1-oxide; |
| 45 | (S)-3-cyano-5-(((4-((2-(hydroxymethyl)piperidin-1-yl)methyl)-7- |
| | ((2-methyl-[1,1′-biphenyl]-3-yl)methoxy)-2,3-dihydro-1H-inden- |
| | 5-yl)oxy)methyl)pyridine 1-oxide; |
| 46 | (S)-5-(((7-((4′-fluoro-2-methyl-[1,1′-biphenyl]-3-yl)methoxy)-4- |
| | ((2-(hydroxymethyl)piperidin-1-yl)methyl)-2,3-dihydro-1H- |
| | inden-5-yl)oxy)methyl)nicotinonitrile; |
| 47 | (S)-5-(((7-((4′-fluoro-2-methyl-[1,1′-biphenyl]-3-yl)methoxy)-4- |
| | ((6-(hydroxymethyl)-5-azaspiro[2.4]heptan-5-yl)methyl)-2,3- |
| | dihydro-1H-inden-5-yl)oxy)methyl)nicotinonitrile; |
| 48 | (S)-5-(((7-((4′-fluoro-2-methyl-[1,1′-biphenyl]-3-yl)methoxy)-4- |
| | ((2-(hydroxymethyl)pyrrolidin-1-yl)methyl)-2,3-dihydro-1H- |
| | inden-5-yl)oxy)methyl)nicotinonitrile; |
| 49 | 5-((5-cyanopyridin-3-yl)methoxy)-7-((2-methyl-[1,1′-biphenyl]- |
| | 3-yl)methoxy)-N-(1-methylpiperidin-4-yl)-2,3-dihydro-1H- |
| | indene-4-carboxamide; |
| 50 | (S)-5-(((4-(2-(hydroxymethyl)pyrrolidine-1-carbonyl)-7-((2- |
| | methyl-[1,1′-biphenyl]-3-yl)methoxy)-2,3-dihydro-1H-inden-5- |
| | yl)oxy)methyl)nicotinonitrile; |
| 51 | (S)-5-(((4-(3-(hydroxymethyl)pyrrolidine-1-carbonyl)-7-((2- |
| | methyl-[1,1′-biphenyl]-3-yl)methoxy)-2,3-dihydro-1H-inden-5- |
| | yl)oxy)methyl)nicotinonitrile; |
| 52 | N-((1-((5-((5-cyanopyridin-3-yl)methoxy)-7-((2-methyl-[1,1′- |
| | biphenyl]-3-yl)methoxy)-2,3-dihydro-1H-inden-4- |
| | yl)methyl)pyrrolidin-2-yl)methyl)acetamide; |
| 53 | 5-(((4-((2-(aminomethyl)pyrrolidin-1-yl)methyl)-7-((2-methyl- |
| | [1,1′-biphenyl]-3-yl)methoxy)-2,3-dihydro-1H-inden-5- |
| | yl)oxy)methyl)nicotinonitrile; |
| 54 | 5-(((7-((3-bromo-2-methylbenzyl)oxy)-4-((2- |
| | (hydroxymethyl)azetidin-1-yl)methyl)-2,3-dihydro-1H-inden-5- |
| | yl)oxy)methyl)nicotinonitrile; |
| 55 | 5-(((7-((4′-hydroxy-2-methyl-[1,1′-biphenyl]-3-yl)methoxy)-4- |
| | ((2-(hydroxymethyl)azetidin-1-yl)methyl)-2,3-dihydro-1H- |
| | inden-5-yl)oxy)methyl)nicotinonitrile; |
| 56 | 5-(((7-((2′-fluoro-2,5′-dimethyl-[1,1′-biphenyl]-3-yl)methoxy)-4- |
| | ((2-(hydroxymethyl)azetidin-1-yl)methyl)-2,3-dihydro-1H- |
| | inden-5-yl)oxy)methyl)nicotinonitrile; |
| 57 | 5-(((4-((2-(hydroxymethyl)azetidin-1-yl)methyl)-7-((2-methyl-3- |
| | (1H-pyrazol-4-yl)benzyl)oxy)-2,3-dihydro-1H-inden-5- |
| | yl)oxy)methyl)nicotinonitrile; |
| 58 | 5-(((4-((2-(hydroxymethyl)azetidin-1-yl)methyl)-7-((4′-methoxy- |
| | 2,2′-dimethyl-[1,1′-biphenyl]-3-yl)methoxy)-2,3-dihydro-1H- |
| | inden-5-yl)oxy)methyl)nicotinonitrile; |
| 59 | 5-(((4-((2-(hydroxymethyl)azetidin-1-yl)methyl)-7-((2-methyl-3- |
| | (pyrimidin-5-yl)benzyl)oxy)-2,3-dihydro-1H-inden-5- |
| | yl)oxy)methyl)nicotinonitrile; |
| 60 | 5-(((4-((2-(hydroxymethyl)azetidin-1-yl)methyl)-7-((2-methyl- |
| | 2′-(trifluoromethyl)-[1,1′-biphenyl]-3-yl)methoxy)-2,3-dihydro- |
| | 1H-inden-5-yl)oxy)methyl)nicotinonitrile; |
| 61 | 5-(((4-((2-(hydroxymethyl)azetidin-1-yl)methyl)-7-((2-methyl-3- |
| | (6-morpholinopyridin-3-yl)benzyl)oxy)-2,3-dihydro-1H-inden-5- |
| | yl)oxy)methyl)nicotinonitrile; |
| 62 | 5-(((7-((2′-fluoro-2-methyl-[1,1′-biphenyl]-3-yl)methoxy)-4-((2- |
| | (hydroxymethyl)azetidin-1-yl)methyl)-2,3-dihydro-1H-inden-5- |
| | yl)oxy)methyl)nicotinonitrile; |
| 63 | 5-(((4-((2-(hydroxymethyl)azetidin-1-yl)methyl)-7-((4′-methoxy- |
| | 2-methyl-[1,1′-biphenyl]-3-yl)methoxy)-2,3-dihydro-1H-inden- |
| | 5-yl)oxy)methyl)nicotinonitrile; |
| 64 | 5-(((7-((3′-fluoro-2-methyl-[1,1′-biphenyl]-3-yl)methoxy)-4-((2- |
| | (hydroxymethyl)azetidin-1-yl)methyl)-2,3-dihydro-1H-inden-5- |
| | yl)oxy)methyl)nicotinonitrile; |
| 65 | 5-(((4-((2-(hydroxymethyl)azetidin-1-yl)methyl)-7-((3′-methoxy- |
| | 2-methyl-[1,1′-biphenyl]-3-yl)methoxy)-2,3-dihydro-1H-inden- |
| | 5-yl)oxy)methyl)nicotinonitrile; |
| 66 | 5-(((4-((2-(hydroxymethyl)azetidin-1-yl)methyl)-7-((2-methyl- |
| | 4′-(trifluoromethoxy)-[1,1′-biphenyl]-3-yl)methoxy)-2,3-dihydro- |
| | 1H-inden-5-yl)oxy)methyl)nicotinonitrile; |
| 67 | 5-(((7-((3′-hydroxy-2-methyl-[1,1′-biphenyl]-3-yl)methoxy)-4- |
| | ((2-(hydroxymethyl)azetidin-1-yl)methyl)-2,3-dihydro-1H- |
| | inden-5-yl)oxy)methyl)nicotinonitrile; |
| 68 | 5-(((4-((2-(hydroxymethyl)azetidin-1-yl)methyl)-7-((2-methyl-3- |
| | (1-methyl-1H-pyrazol-4-yl)benzyl)oxy)-2,3-dihydro-1H-inden-5- |
| | yl)oxy)methyl)nicotinonitrile; |
| 69 | 5-(((4-((2-(hydroxymethyl)azetidin-1-yl)methyl)-7-((2-methyl-3- |
| | (quinolin-8-yl)benzyl)oxy)-2,3-dihydro-1H-inden-5- |
| | yl)oxy)methyl)nicotinonitrile; |
| 70 | 5-(((4-((2-(hydroxymethyl)azetidin-1-yl)methyl)-7-((2-methyl-3- |
| | (pyridin-4-yl)benzyl)oxy)-2,3-dihydro-1H-inden-5- |
| | yl)oxy)methyl)nicotinonitrile; |
| 71 | 5-(((4-((2-(hydroxymethyl)azetidin-1-yl)methyl)-7-((2-methyl-3- |
| | (pyridin-3-yl)benzyl)oxy)-2,3-dihydro-1H-inden-5- |
| | yl)oxy)methyl)nicotinonitrile; |
| 72 | (S)-5-(((4-((2-(hydroxymethyl)azetidin-1-yl)methyl)-7-((2- |
| | methyl-3-(1H-pyrrolo[2,3-b]pyridin-5-yl)benzyl)oxy)-2,3- |
| | dihydro-1H-inden-5-yl)oxy)methyl)nicotinonitrile; |
| 73 | 5-(((7-((2,4′-dimethyl-[1,1′-biphenyl]-3-yl)methoxy)-4-((2- |
| | (hydroxymethyl)azetidin-1-yl)methyl)-2,3-dihydro-1H-inden-5- |
| | yl)oxy)methyl)nicotinonitrile; |
| 74 | 5-(((4-((2-(hydroxymethyl)azetidin-1-yl)methyl)-7-((2-methyl-3- |
| | (1H-pyrrolo[2,3-b]pyridin-5-yl)benzyl)oxy)-2,3-dihydro-1H- |
| | inden-5-yl)oxy)methyl)nicotinonitrile 2,2,2-trifluoroacetate; |
| 75 | 5-(((7-((4′-(tert-butyl)-2-methyl-[1,1′-biphenyl]-3-yl)methoxy)-4- |
| | ((2-(hydroxymethyl)azetidin-1-yl)methyl)-2,3-dihydro-1H- |
| | inden-5-yl)oxy)methyl)nicotinonitrile; |
| 76 | 5-(((7-((4′-fluoro-2-methyl-[1,1′-biphenyl]-3-yl)methoxy)-4-((2- |
| | (hydroxymethyl)azetidin-1-yl)methyl)-2,3-dihydro-1H-inden-5- |
| | yl)oxy)methyl)nicotinonitrile; |
| 77 | 5-(((7-((3-(6-aminopyridin-3-yl)-2-methylbenzyl)oxy)-4-((2- |
| | (hydroxymethyl)azetidin-1-yl)methyl)-2,3-dihydro-1H-inden-5- |
| | yl)oxy)methyl)nicotinonitrile; |
| 77A | (S)-5-(((7-((3-(6-aminopyridin-3-yl)-2-methylbenzyl)oxy)-4-((2- |
| | (hydroxymethyl)azetidin-1-yl)methyl)-2,3-dihydro-1H-inden-5- |
| | yl)oxy)methyl)nicotinonitrile; |
| 78 | (1-((7-((2-methyl-[1,1′-biphenyl]-3-yl)methoxy)-5-phenoxy-2,3- |
| | dihydro-1H-inden-4-yl)methyl)azetidin-2-yl)methanol; |
| 79 | (1-((7-((2-methyl-[1,1′-biphenyl]-3-yl)methoxy)-5-(pyrazin-2- |
| | ylmethoxy)-2,3-dihydro-1H-inden-4-yl)methyl)azetidin-2- |
| | yl)methanol; |
| 80 | 1-((1-((5-((5-cyanopyridin-3-yl)methoxy)-7-((2-methyl-[1,1′- |
| | biphenyl]-3-yl)methoxy)-2,3-dihydro-1H-inden-4- |
| | yl)methyl)pyrrolidin-2-yl)methyl)-3-methylurea formate; and |
| 81 | (1-((7-((2-methyl-[1,1′-biphenyl]-3-yl)methoxy)-5-((1- |
| | methylpiperidin-4-yl)methoxy)-2,3-dihydro-1H-inden-4- |
| | yl)methyl)azetidin-2-yl)methanol. |

or stereoisomers thereof, an N-oxide or a pharmaceutically acceptable salts thereof.

7. The compound as claimed in claim 1, their stereoisomers, an N-oxide or pharmaceutically acceptable salts thereof, wherein the compound acts as inhibitors for PD1/PD-L1 interaction.

8. A process for preparation of compounds of Formula (I) as claimed in claim 1, their stereoisomers, an N-oxide or pharmaceutically acceptable salts thereof, comprising the steps of: (a) reacting compounds of Formula (Ia) with a compound A in the presence of a reducing agent and a solvent to obtain compounds of Formula (I):

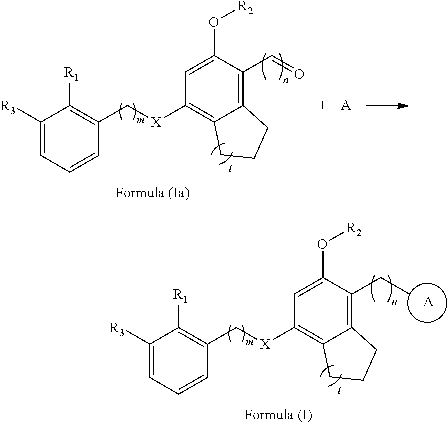

9. The process as claimed in claim 8, wherein the process is carried out at a temperature in the range of 25 to 80° C. for a time period in the range of 2 hours to 20 hours; the reducing agent is selected from sodium cyanoborohydride, sodium triacetoxyborohydride, or sodium borohydride and the solvent is selected from methanol, ethanol, dimethyl formamide or combinations thereof.

10. The process as claimed in claim 8, wherein the Formula (I) optionally reacted with potassium tertiary butoxide in the presence of a solvent selected from tetrahydrofuran, t-butanol or combinations thereof.

11. A pharmaceutical composition comprising a compound of Formula (I) or a pharmaceutically acceptable salt thereof as claimed in claim 1, together with a pharmaceutically acceptable carrier, optionally in combination with one or more other pharmaceutical compositions.

12. A method for treatment and/or prevention of a condition mediated by PD-1/PD-L1 or a proliferative disorder or cancer, the method comprising administering to a subject suffering from a condition mediated by PD-1/PD-L1 interaction or the proliferative disorder or cancer, a therapeutically effective amount of the compound as claimed in claim 1.

13-16. (canceled)

17. A method for treatment of cancer, said method comprising administering to a subject in need thereof a combination of the compounds as claimed in claim 1, with other clinically relevant cytotoxic agents, non-cytotoxic agents, or immune modulators agents to a subject in need thereof.

18. (canceled)

19. The method as claimed in claim 12, wherein the condition mediated by PD-1/PD-L1 or a proliferative disorder or cancer is selected from metastasis cancer, breast cancer, prostate cancer, pancreatic cancer, gastric cancer, lung cancer, colon cancer, rectal cancer, esophagus cancer, duodenal cancer, tongue cancer, pharyngeal cancer, brain tumor, neurinoma, clear cell carcinoma, non-small cell lung cancer, small cell lung cancer, liver cancer, kidney cancer, Hodgkin's lymphoma, head and neck cancer, urothelial cancer, bile duct cancer, uterine body cancer, cervical cancer, ovarian cancer, urinary bladder cancer, skin cancer, hemangioma, malignant lymphoma, malignant melanoma, thyroid cancer, bone tumor, vascular fibroma, glioblastoma, neuroblastoma, hepatoblastoma, medulloblastoma, nephroblastoma, pancreatoblastoma, pleuropulmonary blastoma, sarcoma, neuroendocrine tumors, retinoblastoma, penile cancer, pediatric solid cancer, renal cell carcinoma, lymphoma, myeloma, leukemia, acute myelogenous leukemia (AML), chronic myelogenous leukemia (CML), chronic neutrophilic leukemia, chronic eosinophilic leukemia, chronic lymphocytic leukemia (CLL), acute lymphoblastic leukemia (ALL), hairy cell leukemia, cutaneous T-cell lymphoma (CTCL), multiple myeloma (MM), metastatic cancer, Myeloproliferative neoplasms (MPN), a disease category that includes polycythemia vera (PV), essential thrombocythemia, essential thrombocytosis (ET) and myelofibrosis (MF), cancers with mutations in specific oncogenes, EGFR, KRAS, or RET.

20. The method for treatment and/or prevention of a condition mediated by PD-1/PD-L1 or a proliferative disorder or cancer as claimed in claim 12, further comprising administering other clinically relevant cytotoxic agents or non-cytotoxic agents to the subject in need thereof.

21. A method for treatment and/or prevention of a condition mediated by PD-1/PD-L1 or a proliferative disorder or cancer, the method comprising administering to a subject suffering from a condition mediated by PD-1/PD-L1 interaction or the proliferative disorder or cancer, a therapeutically effective amount of the pharmaceutical composition as claimed in claim 11.

22. The method for treatment and/or prevention of a condition mediated by PD-1/PD-L1 or a proliferative disorder or cancer as claimed in claim 21, further comprising administering other clinically relevant cytotoxic agents or non-cytotoxic agents to the subject in need thereof.

23. A method for treatment of cancer, said method comprising administering to a subject in need thereof the pharmaceutical composition as claimed in claim 11, with other clinically relevant cytotoxic agents, non-cytotoxic agents, or immune modulators agents to a subject in need thereof.
